(19) United States
(12) Patent Application Publication
(10) Pub. No.: US 2010/0093842 A1
(43) Pub. Date: Apr. 15, 2010

(54) Laulimalide Analogs and Uses Thereof

(75) Inventors: Brian M. Gallagher, JR., Merrimac, MA (US); Charles Johannes, Newbury, MA (US); Xiang-Yi Li, Andover, MA (US); Marc Pesant, Saint-Colomban (CA); Hongjuan Zhao, Lexington, MA (US); Kozo Akasaka, Belmont, MA (US); Francis G. Fang, Andover, MA (US)

Correspondence Address: MYERS BIGEL SIBLEY & SAJOVEC, PO BOX 37428, RALEIGH, NC, 27627, US

(73) Assignee: EISAI CO. LTD.

(21) Appl. No.: 12/548,002

(22) Filed: Aug. 26, 2009

Related U.S. Application Data
(63) Continuation of application No. 10/572,870, filed on May 9, 2007, now abandoned.
(60) Provisional application No. 60/505,354, filed on Sep. 23, 2003.

(51) Int. Cl. A61K 31/365; C07D 493/14; C07D 493/04; C12N 5/00; A61P 35/00

(52) U.S. Cl. 514/450; 549/267; 435/375

(57) ABSTRACT

The present invention provides compounds having formula I:

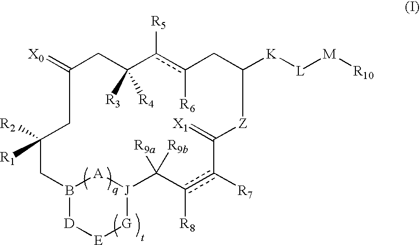

and pharmaceutically acceptable derivatives thereof, wherein R₁-R₁₀, q, t, X₀, X₁, A, B, D, E, G, J, K, L, M and Z are as described generally and in classes and subclasses herein, and additionally provides pharmaceutical compositions thereof, and methods for the use thereof for the treatment of disorders associated with cellular hyperproliferation.

### PRIORITY CLAIM

The present Application claims priority to U.S. Provisional Patent Application No. 60/505,354, filed Sep. 23, 2003; the entire contents of which are incorporated herein by reference.

### BACKGROUND OF THE INVENTION

Neoplastic diseases or cancers, characterized by the proliferation of cells not subject to normal growth regulation, are a major cause of death in humans. An estimated 1,334,100 new cases and 556,500 deaths are expected to occur in 2003. Lung cancer remains the leading cause of cancer-related deaths in the United States; the estimated 157,200 deaths would account for 28% of the total.

Clinical experience in chemotherapy has demonstrated that new and more effective cytotoxic drugs are desirable to treat these diseases. Such experience has also demonstrated that drugs which disrupt the microtubule system of the cytoskeleton can be effective in inhibiting the proliferation of neoplastic cells.

Paclitaxel (more commonly known as Taxol™) and related taxanes have been shown to inhibit microtubule dynamics. These compounds, taxanes, are now recognized as a new class of anti-cancer compounds. Specifically, Paclitaxel is currently employed as a first-line chemotherapeutic agent; however, concerns for its therapeutic index and formulation difficulties, due to its insolubility in water, are a liability: Paclitaxel can be administered effectively only in a solvent including cremophor, which combination can provoke severe hypersensitive immune responses. In addition, taxanes lack or display reduced activity against drug-resistant tumors and cells. Since the discovery of the mechanism of action of paclitaxel, three other non-taxane chemical classes (epothilones A and B, discodermolide, and eleutherobin and related sarcodictyins A and B) have subsequently been identified to possess a similar mode of action. Laulimalide and naturally-occurring analogues thereof have since joined this group.

Laulimalide (1) and Isolaulimalide (2), also known as fijianolides, were originally isolated from the Indonesian sponge Hyatella sp. (Crews P. et al., “Fijianolides, polyketide heterocycles from a marine sponge,” J. Org. Chem., 1988, 53, 3642; D. G. Corley et al., “Laulimalides. New potent cytotoxic macrolides from a marine sponge and a nudibranch predator,” J. Org. Chem. 1988, 53, 3644-3646), and later found along with Neolaulimalide (3) in the Okinawan sponge Fasciospongia rimosa (Jefford et al., “Structures and absolute configurations of the marine toxins, latrunculin A and Laulimalide,” 1996, Tetrahedron Lett. 37: 159-162; Higa et al., “Three new cytotoxic macrolides from a marine sponge,” PCT publication No. WO 97/10242). The absolute structure of natural (−)-Laulimalide has been determined by X-ray crystallography.

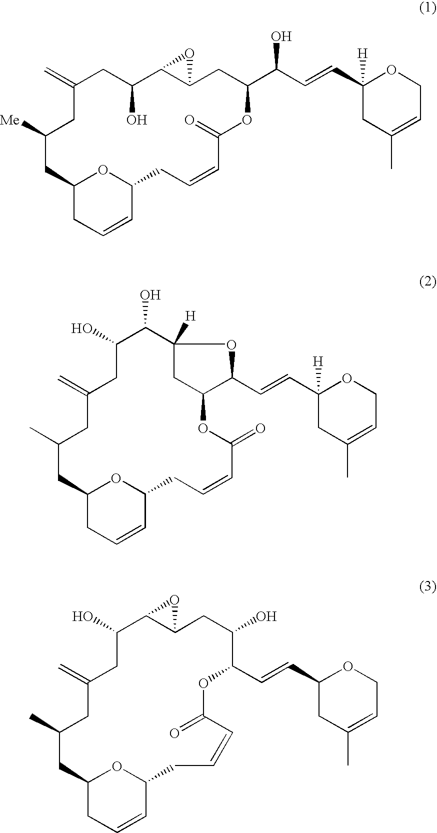

Laulimalide was shown to possess potent cytotoxic activity toward several cancer lines (See, for example, Crews P. et al. and Corley et al. references above; Tanaka J.-I. et al., Chem. Lett., 1996, 255; Jefford C. W. et al., Tetrahedron Lett., 1996, 37, 159). It was later reported that Laulimalide apparently functioned through a similar mechanism of action to that of paclitaxel, the epothilones, eleutherobin and discodermolide: a mechanism involving inhibition of cellular division by stabilization of microtubule assemblies, thereby leading to cell death (See, Mooberry S. L. et al., Cancer Res., 1999, 59, 653). In addition to its potent in vitro anti-mitotic activity (5-12 nM), Laulimalide was also reported to show activity against multi-drug resistant (MDR) cell lines that over express the P-glycoprotein pump (P-gp).

In light of the potential therapeutic utility of Laulimalide and some of its analogues, it would be desirable to develop synthetic methodologies to access and investigate the therapeutic effect of a variety of novel analogues of Laulimalide. In particular, given the interest in the potential therapeutic utility of this class of compounds, it would also be desirable to develop methodologies capable of providing significant quantities of Laulimalide and analogues, for clinical trials and for large-scale preparation.

### SUMMARY OF THE INVENTION

As discussed above, there remains a need for the development of novel Laulimalide analogs and the evaluation of their biological activity. The present invention provides novel compounds of general formula I:

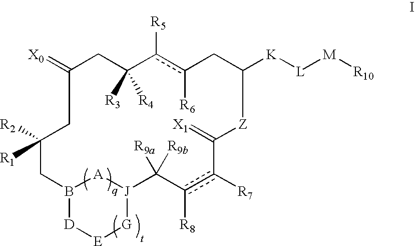

and pharmaceutical compositions thereof, as described generally and in subclasses herein, which compounds are useful as microtubule stabilizing agents. Thus these compounds are useful, for example, for the treatment of various disorders including cancer and disorders associated with cellular hyperproliferation such as many inflammatory disorders, for example psoriasis, eczema, dermatitis, multiple sclerosis, and rheumatoid arthritis, and restenosis. The compounds of the invention have further utility to kill cells, ameliorate the detrimental effects of cell growth, and generally to substitute for any other cytotoxic agent in any application thereof.

In yet another aspect, the present invention provides methods for treating or lessening the severity of disorders associated with cellular hyperproliferation comprising administering to a subject in need thereof a therapeutically effective amount of the compound of the invention. In yet another aspect, the present invention provides methods for treating or lessening the severity of cancer comprising administering to a subject in need thereof a therapeutically effective amount of the compound of the invention in an amount effective to inhibit cell proliferation. In yet another aspect, the present invention provides methods for treating or lessening the severity of inflammatory disorders comprising administering to a subject in need thereof a therapeutically effective amount of the compound of the invention in an amount effective to inhibit cell proliferation.

### DEFINITIONS

The tell “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments, the term “heteroaromatic moiety” refers to a planar ring comprising at least on eheteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; hetero aromatic; aryl; heteroaryl; alkylaryl; heteroalkylaryl; alkylheteroaryl; hetero alkylhetero aryl ; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substitutents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₁₋₆alkylidene”, as used herein, refers to a substituted or unsubstituted, linear or branched saturated divalent radical consisting solely of carbon and hydrogen atoms, having from one to six carbon atoms, having a free valence “-” at both ends of the radical.

The term “C₂₋₆alkenylidene”, as used herein, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the tetras “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### DETAILED DESCRIPTION OF CERTAIN PREFERRED EMBODIMENTS OF THE INVENTION

In recognition of the need to access and further explore the biological activity of novel Laulimalide analogs, and this class of macrocycles in general, the present invention provides novel macrocyclic compounds, as described in more detail herein, which exhibit the ability to stabilize microtubules. Thus, the compounds of the invention, and pharmaceutical compositions thereof, are useful as microtubule stabilizing agents for the treatment of cancer and/or disorders associated with cell hyperproliferation. In certain embodiments, the compounds of the present invention can be used for the treatment of diseases and disorders including, but not limited to solid tumor cancers, inflammatory disorders, for example psoriasis, eczema, dermatitis, multiple sclerosis, and rheumatoid arthritis, and restenosis, to name a few.

1) General Description of Compounds of the Invention

The compounds of the invention include compounds of the general formula I as further defined below:

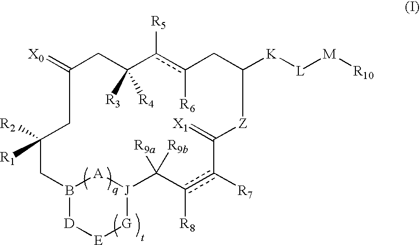

pharmaceutically acceptable derivatives thereof;

wherein R₁ and R₂ are independently hydrogen, halogen, or an aliphatic, alicyclic, heteroaliphatic, heteroalicyclic, aromatic or heteroaromatic moiety;

R₃ and R₄ are independently hydrogen, —OR^{3a} or —NR^{3a}R^{3b}, wherein at least one of R₃ and R₄ is —OR^{3a} or —NR^{3a}R^{3b}, or R₃ and R₄ taken together with the carbon to which they are attached form a —C(═O)— or ═NR^{3c} moiety; wherein R^{3a} and R^{3b}, for each occurrence, is independently hydrogen, a protecting group, a prodrug moiety or an aliphatic, alicyclic, heteroaliphatic, heteroalicyclic, aromatic or heteroaromatic moiety; and R^{3c}is an aliphatic, alicyclic, heteroaliphatic, heteroalicyclic, aromatic or heteroaromatic moiety, or OR^{3d}; wherein R^{3d} is hydrogen or an aliphatic, alicyclic, heteroaliphatic, heteroalicyclic, aromatic or heteroaromatic moiety;

R₅ and R₆ are independently hydrogen, halogen, —CN, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form an alicyclic or heteroalicyclic moiety; wherein the carbon atoms to which R₅ and R₆ are attached may be connected by a single or double bond, as valency permits; and wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or R₆, taken together with a substituent present on K, forms an alicyclic, heterocyclic, aromatic or heteroaromatic moiety;

R₇ and R₈ are independently absent, hydrogen, halogen, —CN, or an aliphatic, alicyclic, heteroaliphatic, heteroalicyclic, aromatic or heteroaromatic moiety, or R₇ and R₈, taken together, form an alicyclic, heteroalicyclic, aromatic or heteroaromatic moiety; wherein the carbon atoms to which R₇ and R₈ are attached may be connected by a single, double or triple bond, as valency permits;

R₉ₐ and R_{9b} are independently absent, hydrogen or an aliphatic, alicyclic, heteroaliphatic, heteroalicyclic, aromatic or heteroaromatic moiety, or one of R₉ₐ and R_{9b}, taken together with X₁, form an alicyclic, heteroalicyclic, aromatic or heteroaromatic moiety;

R₁₀ is hydrogen or an aliphatic, alicyclic, heteroaliphatic, heteroalicyclic, aromatic or heteroaromatic moiety;

X₀ is CR^{X0a}R^{X0b}, O or NR^{X0a}; wherein R^{X0a} and R^{X0b} are independently hydrogen, a nitrogen protecting group, or an aliphatic, alicyclic, heteroaliphatic, heteroalicyclic, aryl or heteroaryl moiety;

X₁ is O, S or NR^{X1}, or X₁, taken together with one of R₉ₐ and R_{9b}, forms an alicyclic, heteroalicyclic, aromatic or heteroaromatic moiety; wherein R^{X1} is hydrogen, a nitrogen protecting group, or an aliphatic, alicyclic, heteroaliphatic, heteroalicyclic, aromatic or heteroaromatic moiety;

Z is O, NR^{Z1}, CR^{Z1}R^{Z2}or S, wherein R^{Z1} and R^{Z2} are independently hydrogen, halogen, a nitrogen protecting group, or an aliphatic, alicyclic, heteroaliphatic, heteroalicyclic, aromatic or heteroaromatic moiety;

K, L and M are independently absent, or a substituted or unsubstituted C₁₋₆alkylidene or C₂₋₆alkenylidene chain wherein up to two non-adjacent methylene units are independently optionally replaced by CO, CO₂, COCO, CONR^{P1}, OCONR^{P1}, NR^{P1}NR^{P2}, NR^{P1}NR^{P2}CO, NR^{P1}CO, NR^{P1}CO₂, NR^{P1}CONR^{P2}, SO, SO₂, NR^{P1}SO₂, SO₂NR^{P1}, NR^{P1}SO₂NR^{P2}, O, S, or NR^{P1}; wherein each occurrence of R^{P1} and R^{P2} is independently hydrogen, aliphatic, heteroaliphatic, aromatic, heteroaromatic or acyl, or a substitutent present on K, when present, and taken together with R₆, forms an alicyclic, heterocyclic, aromatic or heteroaromatic moiety;

A, B, D, E, G and J are independently connected by either a single or double bond, as valency permits, or A-B-D-E-G-J together represents an aromatic or heteroaromatic moiety; wherein B and J are independently N or CR^{Q1}; and A, D, E and G are independently C═O, CR^{Q1}R^{Q2}, NR^{Q1}, O, N or S; wherein each occurrence of R^{Q1} and R^{Q2} is independently absent, hydrogen, halogen, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or any two adjacent substituents on A, B, D, E, G and J, taken together, may represent an alicyclic, heteroalicyclic, aromatic or heteroaromatic moiety; and

q and t are independently 0-2; wherein the sum q+t is 1-3.

In certain embodiments, compounds of formula (I) exclude compounds having the following structure:

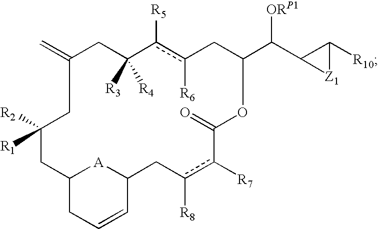

wherein one of R₁ and R₂ is hydrogen, the other is α-alkyl, β-alkyl or trifluoromethyl;

R^{3a} is hydrogen, α-alkyl, β-alkyl, methylmethylether (methoxymethyl ether) or alkyl optionally substituted with hydroxy, paramethyloxybenzyl, benzyl or a protecting group;

R₅ and R₆ are each hydrogen and the carbon atoms to which R₅ and R₆ are attached are connected by a cis- or trans-double bond; or, R₅ and R₆, taken together with the carbon atoms to which they are attached, represent a moiety having the structure:

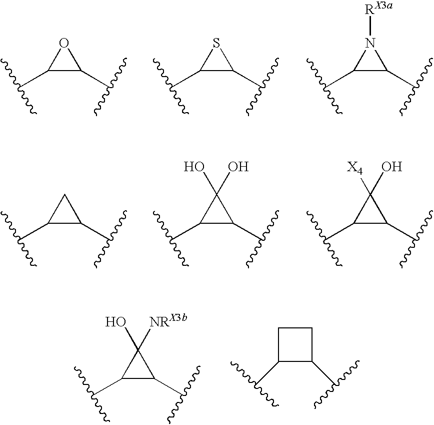

wherein X₄ is a halogen; R^{X3a} is hydrogen, alkyl, cycloalkyl or aryl; and R^{X3b} is alkyl, cycloalkyl or aryl;
R₇ and R₈ are each hydrogen and the carbon atoms to which R₇ and R₈ are attached are connected by a cis- or trans-double bond;
R₁₀ is a 5- or 6-membered optionally substituted aryl or heteroaryl, or an optionally substituted and/or partially saturated 5- or 6-membered ring, which can be interrupted by O, S or NR^{10A}; wherein R^{10A} is hydrogen, alkyl, cycloalkyl or aryl;
Z₁ is a (Z)- or (E)-double bond, triple bond, or is O, S, CH₂, —(CH₂)₂—, ═C(OH)₂, ═C(halogen)(OH), ═C(OH)NR^{X3b} or ═NR^{X3a}; wherein R^{X3a} and R^{X3b} are as defined above;
R^{P2} is hydrogen, α-alkyl, β-alkyl, methylmethylether, alkyl optionally substituted with hydroxy, paramethyloxybenzyl, benzyl or a protecting group; and
A is O, S or NR^{A1}, where R^{A1} is hydrogen, alkyl, cycloalkyl or aryl.

In certain embodiments, compounds of formula (I) exclude compounds having the following structure:

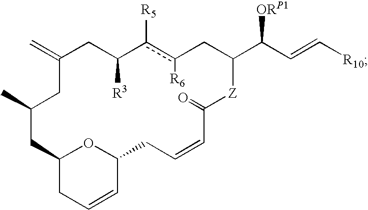

wherein R₃ is hydrogen, hydroxyl, C₁₋₁₀alkoxy, aryloxy or alkylaryloxy;

R₅ and R₆ are each hydrogen and the carbon atoms to which R₅ and R₆ are attached are connected by trans-double bond; or, R₅ and R₆, taken together with the carbon atoms to which they are attached, represent a moiety having the structure:

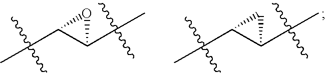

Z is O or NH;

R₁₀ is substituted or unsubstituted cyclohexyl, substituted or unsubstituted 3-cyclohexenyl, substituted or unsubstituted phenyl, substituted or unsubstituted pyridyl, substituted or unsubstituted thiazolyl, or a group of the formula:

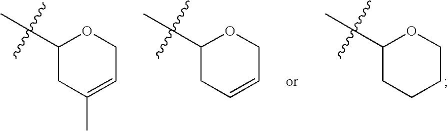

and

R^{P1} is hydrogen, OH or C₁₋₅alkoxy.

In certain embodiments, compounds of formula (I) exclude compounds having the following structures:

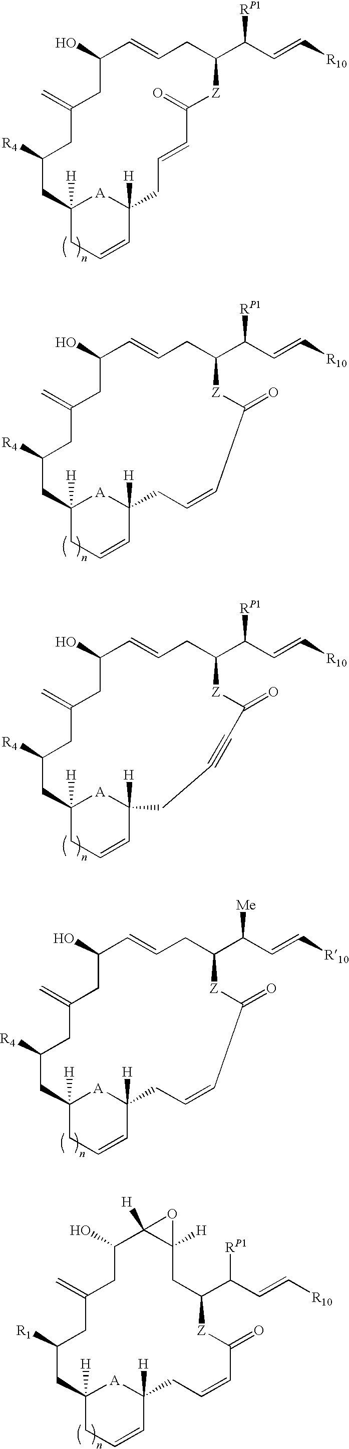

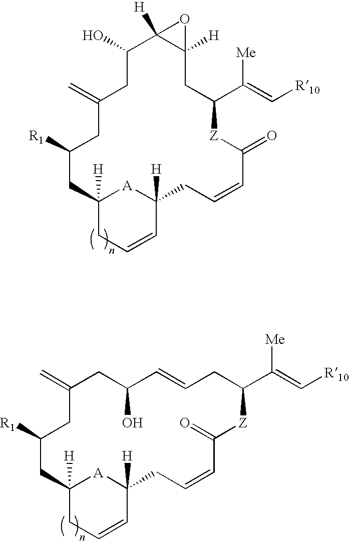

wherein n is 0 or 1;

R₁ is selected from the group consisting of C₁₋₄alkyl, hydroxyl, —OC₁₋₄alkyl, —OC₂₋₄alkenyl, —OC₂₋₄alkynyl, —Oheteoaryl, —Oaryl, —C₃₋₇cycloalkyl, —C₃₋₇heterocycloalkyl, aryl and heteroaryl;

A and Z are independently selected from CH₂, O, S, NH, —NC₁₋₄alkyl, —NC₂₋₄alkenyl, —NC₂₋₄alkynyl, —Nheteoaryl, and —Naryl;

R^{P1} is selected from the group consisting of H, hydroxyl, —OC₁₋₄alkyl, —OC₂₋₄alkenyl, —OC₂₋₄alkynyl, —Oheteoaryl, —Oaryl and C₁₋₃alkyl;

R₁₀ is selected from the group consisting of C₃₋₇heterocycloalkyl, C₃₋₇heterocycloalkenyl, C₃₋₇cycloalkyl, C₃₋₇cycloalkenyl, C₁₋₃alkyleneOR^{10A}, —OR^{10A}, C₁₋₃alkyleneN(R^{10A})2, —N(R^{10A})₂, aryl and heteroaryl; wherein R^{10A} is H, C₁₋₄alkyl, C₂₋₄alkenyl, C₂₋₄alkynyl, heteoaryl or aryl; and

R′₁₀ is selected from the group consisting of heteroaryl, aryl, C₃₋₇heterocycloalkyl and C₃₋₇heterocycloalkenyl.

In certain embodiments, compounds of formula (I) exclude compounds depicted on pages 107-111 and 114 of WO 03/076445.

In certain embodiments, compounds of formula (I) exclude compounds having the following structures:

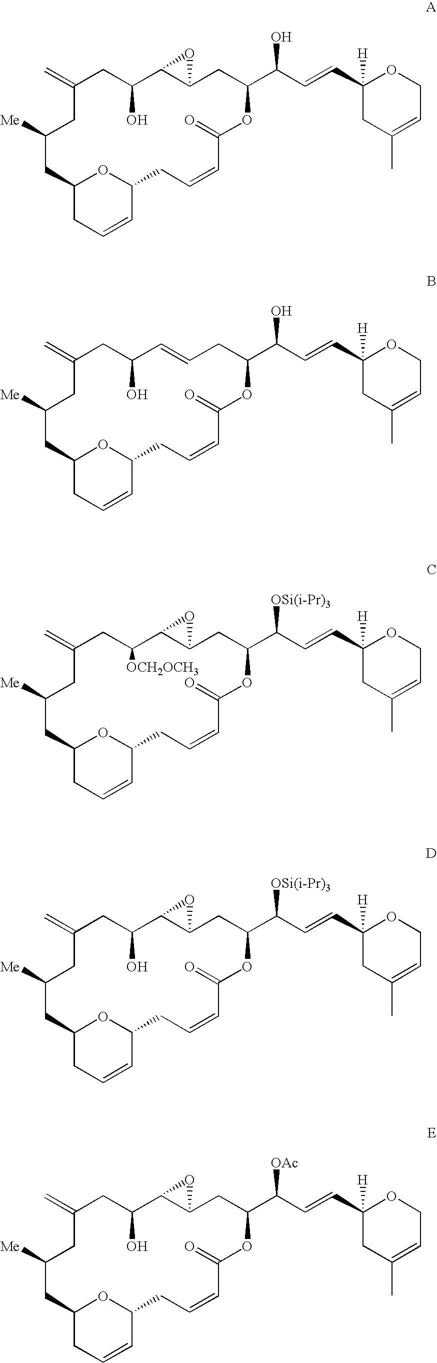

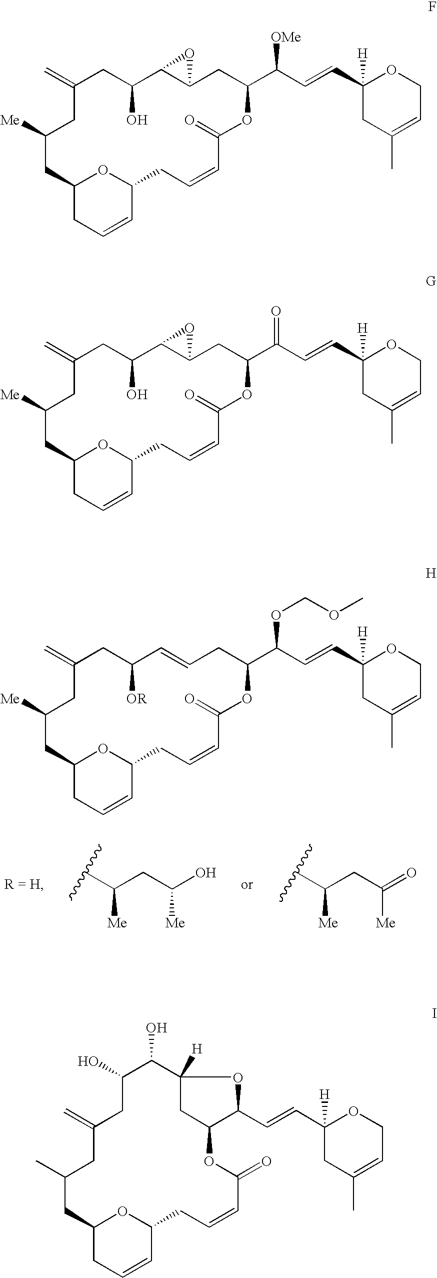

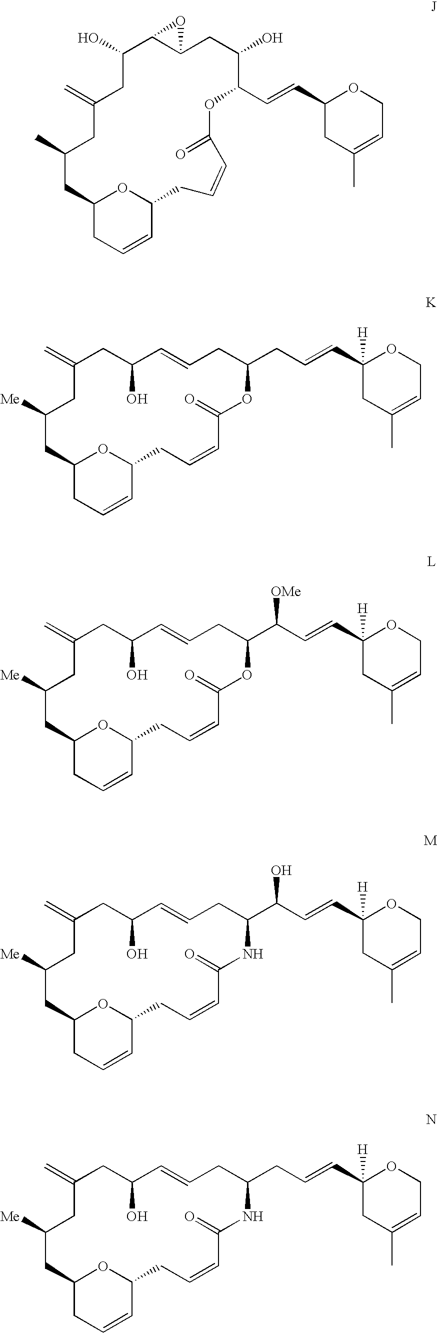

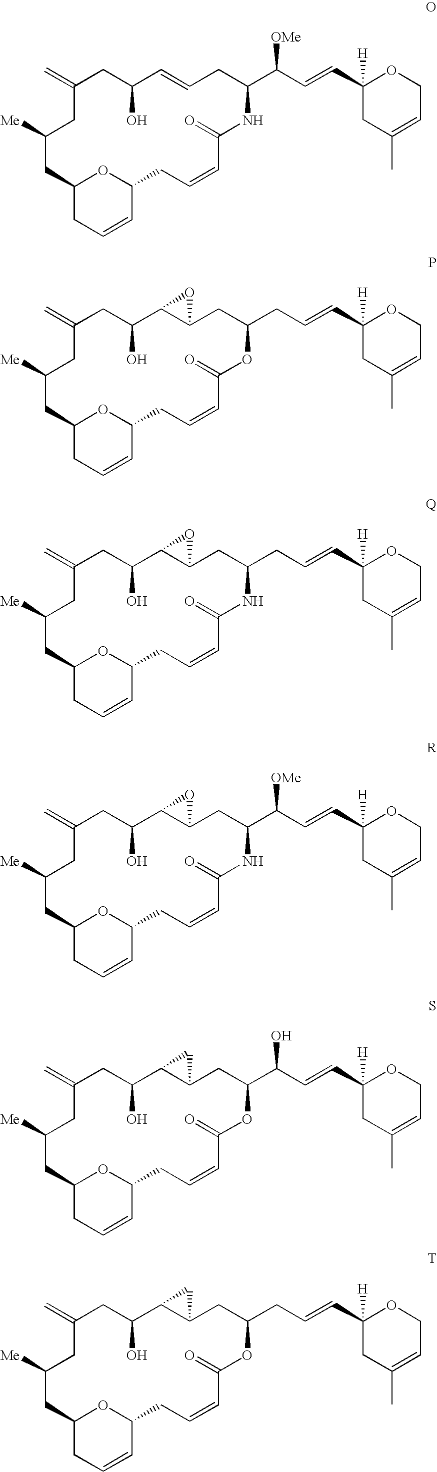

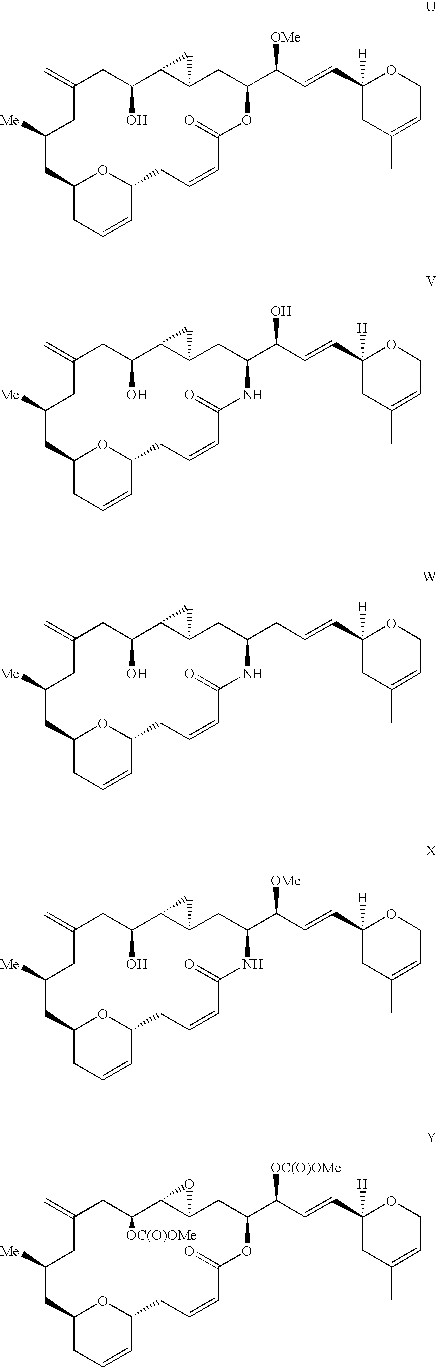

In certain embodiments, the compound is not a compound having one of the structures A, C-D and G-Y.

In certain embodiments, the present invention defines particular classes of compounds which are of special interest. For example, one class of compounds of special interest includes those compounds of Formula I wherein:

R₁ and R₂ are independently hydrogen, halogen, or an alkyl, cycloalkyl, heteroalkyl, heterocyclic, aryl or heteroaryl moiety;

R₃ and R₄ are independently hydrogen, —OR^{3a} or —NR^{3a}R^{3b}, wherein at least one of R₃ and R₄ is —OR^{3a} or —NR^{3a}R^{3b}, or R₃ and R₄ taken together with the carbon to which they are attached form a a —C(═O)— or ═NR^{3c} moiety; wherein R^{3a} and R^{3b}, for each occurrence, is independently hydrogen, a protecting group, a prodrug moiety or an alkyl, cycloalkyl, heteroalkyl, heterocyclic, aryl, heteroaryl, arylalkyl or heteroarylalkyl moiety; and R^{3c} is an alkyl, cycloalkyl, heteroalkyl, heterocyclic, aryl or heteroaryl moiety, or OR^{3d}; wherein R^{3d} is hydrogen or an alkyl, cycloalkyl, heteroalkyl, heterocyclic, aryl or heteroaryl moiety;

R₅ and R₆ are independently hydrogen, halogen, —CN, an alkyl, cycloalkyl, heteroalkyl, heterocyclic, aryl, heteroaryl, arylalkyl or heteroarylalkyl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form a cycloalkyl or heterocyclic moiety; wherein the carbon atoms to which R₅ and R₆ are attached may be connected by a single or double bond, as valency permits; and wherein each occurrence of R^{W1} and R^{W2} is independently hydrogen, a protecting group, a prodrug moiety or an alkyl, cycloalkyl, heteroalkyl, heterocyclic, aryl, heteroaryl, arylalkyl or heteroarylalkyl moiety, or, when W is NR^{W2}, R^{W1} and R^{W2}, taken together with the nitrogen atom to which they are attached, form a heterocyclic or heteroaryl moiety; or R₆, taken together with a substituent present on K, forms an alicyclic, heterocyclic, aryl or heteroaryl moiety;

R₇ and R₈ are independently absent, hydrogen, halogen or an alkyl, cycloalkyl, heteroalkyl, heterocyclic, aryl, heteroaryl, arylalkyl or heteroarylalkyl moiety, wherein the carbon atoms to which R₇ and R₈ are attached may be connected by a single, double or triple bond, as valency permits;

R₉ₐ and R_{9b} are independently absent, hydrogen or an alkyl, cycloalkyl, heteroalkyl, heterocyclic, aryl, heteroaryl, arylalkyl or heteroarylalkyl moiety;

R₁₀ is hydrogen or an alkyl, cycloalkyl, heteroalkyl, heterocyclic, aryl, heteroaryl, arylalkyl or heteroarylalkyl moiety;

X₀ or CR^{X0a}R^{X0b}, O or NR^{X0a}; wherein R^{X0a} and R^{X0b} are independently hydrogen, a nitrogen protecting group, or an alkyl, cycloalkyl, heteroalkyl, heterocyclic, aryl or heteroaryl moiety;

X₁ is O, S or NR^{X1}; wherein R^{X1} is hydrogen, a nitrogen protecting group, or an alkyl, cycloalkyl, heteroalkyl, heterocyclic, aryl, heteroaryl, arylalkyl or heteroarylalkyl moiety;

Z is O, NR^{Z1}, CR^{Z1}R^{Z2} or S, wherein R^{Z1} and R^{Z2} are independently hydrogen, halogen, a nitrogen protecting group, or an alkyl, cycloalkyl, heteroalkyl, heterocyclic, aryl, heteroaryl, arylalkyl or heteroarylalkyl moiety;

K, L and M are independently absent, CR^{P1}R^{P2}, CR^{P1} or C═O, wherein each occurrence of R^{P1} is independently hydrogen, halogen, an alkyl, cycloalkyl, heteroalkyl, heterocyclic, aryl, heteroaryl, arylalkyl or heteroarylalkyl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl, heteroaryl, arylalkyl or heteroarylalkyl moiety, or, when W is NR^{W2}, R^{W1} and R^{W2}, taken together with the nitrogen atom to which they are attached, form a heterocyclic or heteroaryl moiety; or a substitutent present on K, when present, and taken together with R₆, forms an alicyclic, heterocyclic, aromatic or heteroaromatic moiety; and

A, B, D, E, G and J are independently connected by either a single or double bond, as valency permits, or A-B-D-E-G-J together represents an aryl or heteroaryl moiety; wherein B and J are independently N or CR^{Q1}; and A, D, E and G are independently C═O, CR^{Q1}R^{Q2}, NR^{Q1}, O, N or S; wherein each occurrence of R^{Q1} and R^{Q2} is independently absent, hydrogen, halogen, an alkyl, cycloalkyl, heteroalkyl, heterocyclic, aryl, heteroaryl, arylalkyl or heteroarylalkyl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl, heteroaryl, arylalkyl or heteroarylalkyl moiety, or, when W is NR^{W2}, R^{W1} and R^{W2}, taken together with the nitrogen atom to which they are attached, form a heterocyclic or heteroaryl moiety; or any two adjacent substituents on A, B, D, E, G and J, taken together, may represent an alkyl, cycloalkyl, heteroalkyl, heterocyclic, aryl, heteroaryl, arylalkyl or heteroarylalkyl moiety.

Another class of compounds of special interest includes those compounds having the structure:

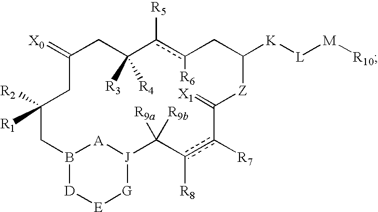

wherein R₁-R₁₀, X₀, X₁, A, B, D, E, G, J, K, L, M and Z are as described generally and in classes and subclasses herein.

Another class of compounds of special interest consists of compounds having the structure:

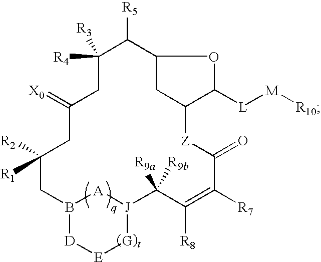

wherein R₁-R₅, R₇-R₁₀, X₀, q, t, A, B, D, E, G, J, L, M and Z are as described generally and in classes and subclasses herein.

A number of important subclasses of each of the foregoing classes deserve separate mention; these subclasses include subclasses of the foregoing classes in which:
i) R₁ and R₂ are independently hydrogen, halogen, or an aliphatic, alicyclic, heteroaliphatic, heteroalicyclic, aryl or heteroaryl moiety;
ii) R₁ and R₂ are independently hydrogen, halogen, or an alkyl, cycloalkyl, heteroalkyl, heterocyclic, aryl or heteroaryl moiety;
iii) R₁ and R₂ are independently hydrogen or an alkyl, cycloalkyl, heteroalkyl, heterocyclic, aryl or heteroaryl moiety;
iv) R₁ and R₂ are independently hydrogen, lower alkyl, or aryl;
v) R₁and R₂ are independently hydrogen, methyl or ethyl;
vi) R₁ and R₂ are each hydrogen;
vii) at least one of R₁ and R₂ is lower alkyl;
viii) at least one of R₁ and R₂ is methyl;
ix) R₁ and R₂ are each independently lower alkyl;
x) R₁ and R₂ are each methyl;
xi) R₃ and R₄ are independently hydrogen, —OR^{3a} or —NR^{3a}R^{3b}, wherein at least one of R₃ and R₄ is —OR^{3a} or —NR^{3a}R^{3b}, or R₃ and R₄ taken together with the carbon to which they are attached faun a —C(═O)— or —C(═NR^{3c})— moiety; wherein R^{3a} and R^{3b}, for each occurrence, is independently hydrogen, a protecting group, a prodrug moiety or an alkyl, cycloalkyl, heteroalkyl, heterocyclic, aryl, heteroaryl, arylalkyl or heteroarylalkyl moiety; and R^{3c} is an alkyl, cycloalkyl, heteroalkyl, heterocyclic, aryl or heteroaryl moiety, or OR^{3d}; wherein R^{3d} is hydrogen or an alkyl, cycloalkyl, heteroalkyl, heterocyclic, aryl or heteroaryl moiety;
xii) R₃ and R₄ are independently hydrogen or OR^{3a}, wherein at least one of R₃ and R₄ is OR^{3a}; wherein R^{3a}, for each occurrence, is independently hydrogen, an oxygen protecting group, a prodrug moiety or an alkyl, cycloalkyl, heteroalkyl, heterocyclic, aryl or heteroaryl moiety;
xiii) R₃ and R₄ are independently hydrogen or OR^{3a}, wherein at least one of R₃ and R₄ is OR^{3a}; wherein R^{3a}, for each occurrence, is hydrogen, an oxygen protecting group or a prodrug moiety;
xiv) R₄ is hydrogen and R₃ is OR^{3a}; wherein R^{3a} is hydrogen, an oxygen protecting group, a prodrug moiety or an alkyl, cycloalkyl, heteroalkyl, heterocyclic, aryl or heteroaryl moiety;
xv) R₄ is hydrogen and R₃ is OR^{3a}; wherein R^{3a} is hydrogen, an oxygen protecting group or a prodrug moiety;
xvi) one of R₃ and R₄ is hydrogen and the other is OH;
xvii) R₃ is OH and R₄ is hydrogen;
xviii) R₃ is hydrogen and R₄ is OH;
xix) R₃ and R₄ are independently hydrogen or —NR^{3a}R^{3b}, wherein at least one of R₃ and R₄ is —NR^{3a}R^{3b}; wherein R^{3a} and R^{3b}, for each occurrence, is independently hydrogen, a nitrogen protecting group, a prodrug moiety or an aliphatic, alicyclic, heteroaliphatic, heteroalicyclic, acyl, aryl or heteroaryl moiety;
xx) R₃ and R₄ are independently hydrogen or —NR^{3a}R^{3b}, wherein at least one of R₃ and R₄ is —NR^{3a}R^{3b}; wherein R^{3a} and R^{3b}, for each occurrence, is independently hydrogen, a nitrogen protecting group or a prodrug moiety;
xxi) R₄ is hydrogen and R₃ is —NR^{3a}R^{3b}; wherein R^{3a} and R^{3b}, for each occurrence, is independently hydrogen, lower alkyl, acyl, aryl, a nitrogen protecting group or a prodrug moiety;
xxii) R₄ is hydrogen and R₃ is —NR^{3a}R^{3b}; wherein R^{3a} and R^{3b}, for each occurrence, is independently hydrogen, a nitrogen protecting group or a prodrug moiety;
xxiii) one of R₃ and R₄ is hydrogen and the other is NH₂;
xxiv) R₃ is NH₂ and R₄ is hydrogen;
xxv) R₃ is hydrogen and R₄ is NH₂;
xxvi) R₃ and R₄ taken together with the carbon to which they are attached form a —C(═O)— moiety;
xxvii) R₃ and R₄ taken together with the carbon to which they are attached form a ═NR^{3c} moiety; wherein R^{3c} is an alkyl, cycloalkyl, heteroalkyl, heterocyclic, aryl or heteroaryl moiety, or OR^{3d}; wherein R^{3d} is hydrogen or an alkyl, cycloalkyl, heteroalkyl, heterocyclic, aryl or heteroaryl moiety;
xxviii) R₃ and R₄ taken together with the carbon to which they are attached form a ═N—OR^{3d} moiety; wherein R^{3d} is hydrogen or an alkyl, cycloalkyl, heteroalkyl, heterocyclic, aryl or heteroaryl moiety;
xxix) R₃ and R₄ taken together with the carbon to which they are attached form a ═N—OR^{3d} moiety; wherein R^{3d} is hydrogen or lower alkyl;
xxx) R₃ and R₄ taken together with the carbon to which they are attached represent an oxime methyl ether moiety having the structure ═N-OMe;
xxxi) R₅ and R₆ are independently hydrogen, halogen, —CN, an alkyl, cycloalkyl, heteroalkyl, heterocyclic, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form a cycloalkyl or heterocyclic moiety; wherein the carbon atoms to which R₅ and R₆ are attached may be connected by a single or double bond, as valency permits; and wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety;
xii) the carbon atoms to which R₅ and R₆ are attached are connected by a double bond;
xxxiii) R₅ and R₆ are each hydrogen;
xxxiv) the carbon atoms to which R₅ and R₆ are attached are connected by a trans-double bond;
xxxv) the carbon atoms to which R₅ and R₆ are attached are connected by a cis-double bond;
xxxvi) R₅ and R₆ and the carbon atoms to which they are attached form a cycloalkyl or heterocyclic moiety;
xxxvii) R₅ and R₆ and the carbon atoms to which they are attached form a moiety having the structure:

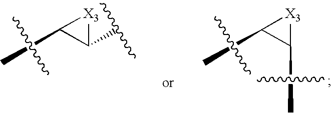

wherein X₃ is CR^{X3a}R^{X3b}, O or NR^{X3a}; wherein R^{X3a} and R^{X3b} are independently hydrogen, a nitrogen protecting group, or an aliphatic, alicyclic, heteroaliphatic, heteroalicyclic, aryl or heteroaryl moiety;
xxxviii) X₃ is CR^{X3a}R^{X3b}, O or NR^{X3a}; wherein R^{X3a} and R^{X3b} are independently hydrogen, a nitrogen protecting group, or an alkyl, cycloalkyl, heteroalkyl, heterocyclic, aryl or heteroaryl moiety;
xxxix) X₃ is CH₂, O or NR^{X3a}; wherein R^{X3a} is hydrogen, a nitrogen protecting group, or an alkyl, cycloalkyl, heteroalkyl, heterocyclic, aryl or heteroaryl moiety;
xl) X₃ is CH₂ or O;
xli) the carbon atoms to which R₅ and R₆ are attached are connected by a single bond;
xlii) R₅ and R₆ are independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an aliphatic, alicyclic, heteroaliphatic, heteroalicyclic, aryl or heteroaryl moiety, or, when W is NR^{W2}, R^{W1} and R^{W2}, taken together with the nitrogen atom to which they are attached, form a heteroalicyclic or heteroaryl moiety;
xliii) R₅ and R₆ are independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety;
xliv) R₅ and R₆ are independently hydrogen, halogen, hydroxyl, OR^{y1} or NR^{y1}R^{y2}; wherein each occurrence of R^{y1} and R^{y2} is independently hydrogen, a protecting group, a prodrug moiety, —C(═O)R^{y3}, or an alkyl, cycloalkyl, heteroalkyl, heterocyclic, aryl or heteroaryl moiety, or R^{y1} and R^{y2}, taken together with the nitrogen atom to which they are attached, form a heterocyclic or heteroaryl moiety; wherein R^{y3} is hydrogen, or an alkyl, cycloalkyl, heteroalkyl, heterocyclic, aryl or heteroaryl moiety;
xlv) R₅ and R₆ are independently hydrogen, Cl, hydroxyl or OR^{y1}; wherein R^{y1} is hydrogen, a protecting group, a prodrug moiety —C(═O)R^{y3}, or an alkyl, cycloalkyl, heteroalkyl, heterocyclic, aryl or heteroaryl moiety; wherein R^{y3} is hydrogen, or an alkyl, cycloalkyl, heteroalkyl, heterocyclic, aryl or heteroaryl moiety;
xlvi) R₆, taken together with a substituent on K, forms a cycloalkyl, heterocyclic, aryl or heteroaryl moiety;
xlvii) R₆, taken together with a substituent on K, forms a 5- to 6-membered cycloalkyl, heterocyclic, aryl or heteroaryl moiety;
xlviii) R₆, taken together with a hydroxyl substituent on K, forms an optionally substituted tetrahydrofuran ring;
xlix) R₇ and R₈ are independently absent, hydrogen, halogen, —CN, or an alkyl, cycloalkyl, heteroalkyl, heterocyclic, aryl or heteroaryl moiety, or R₇ and R₈, taken together, form a cycloalkyl, heterocyclic, aryl or heteroaryl moiety; wherein the carbon atoms to which R₇ and R₈ are attached may be connected by a single, double or triple bond, as valency permits;
l) R₇ and R₈ are independently absent, hydrogen, halogen, —CN, or an alkyl, cycloalkyl, heteroalkyl, heterocyclic, aryl or heteroaryl moiety; wherein the carbon atoms to which R₇ and R₈ are attached may be connected by a single, double or triple bond, as valency permits;
li) the carbon atoms to which R₇ and R₈ are attached are connected by a double bond;
lii) R₇ and R₈ are independently hydrogen, halogen or alkyl;
liii) R₇ and R₈ are independently hydrogen, F or lower alkyl;
liv) R₇ and R₈ are each hydrogen;
lv) the carbon atoms to which R₇ and R₈ are attached are connected by a trans-double bond;
lvi) the carbon atoms to which R₇ and R₈ are attached are connected by a cis-double bond;
lvii) the carbon atoms to which R₇ and R₈ are attached are connected by a triple bond, and R₇ and R₈ are each absent;
lviii) the carbon atoms to which R₇ and R₈ are attached are connected by a single bond, and R₇ and R₈ are independently absent, hydrogen, halogen, —CN, or an alkyl, cycloalkyl, heteroalkyl, heterocyclic, aryl or heteroaryl moiety;
lix) R₇, R₈ and the carbon atoms to which R₇ and R₈ are attached together represent an aryl or heteroaryl moiety;
lx) R₇, R₈ and the carbon atoms to which R₇ and R₈ are attached together represent an optionally substituted phenyl or pyridinyl moiety;
lxi) R₉ₐ and R_{9b} are independently absent, hydrogen or an aliphatic, alicyclic, heteroaliphatic, heteroalicyclic, aryl or heteroaryl moiety;
lxii) R₉ₐ and R_{9b} are independently absent, hydrogen or an alkyl, cycloalkyl, heteroalkyl, heterocyclic, aryl or heteroaryl moiety;
lxiii) R₉ₐ and R_{9b} are each hydrogen;
lxiv) R₉ₐ and R_{9b}, taken together with X₁, forms an optionally substituted alicyclic, heteroalicyclic, aryl or heteroaryl moiety;
lxv) R₉ₐ and R_{9b}, taken together with X₁, forms an optionally substituted phenyl or pyridyl moiety;
lxvi) R₁₀ is hydrogen or an alkyl, cycloalkyl, heteroalkyl, heterocyclic, aryl or heteroaryl moiety;
lxvii) R₁₀ is one of

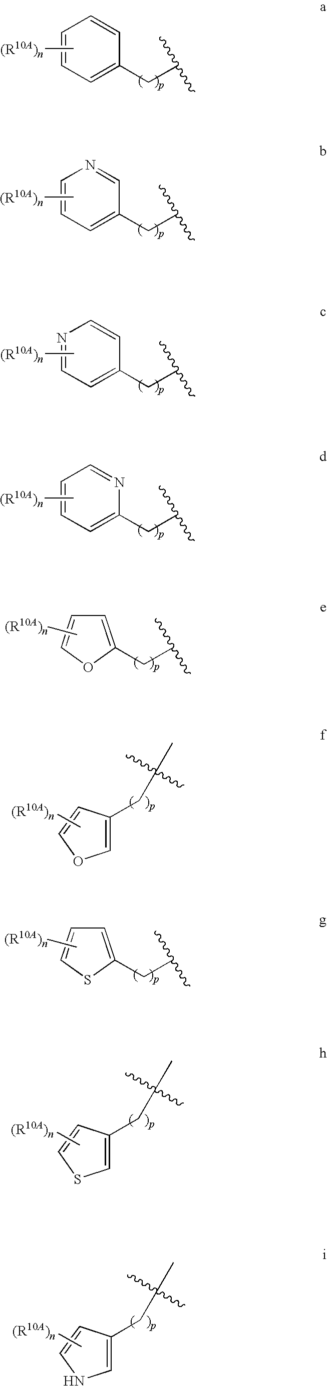

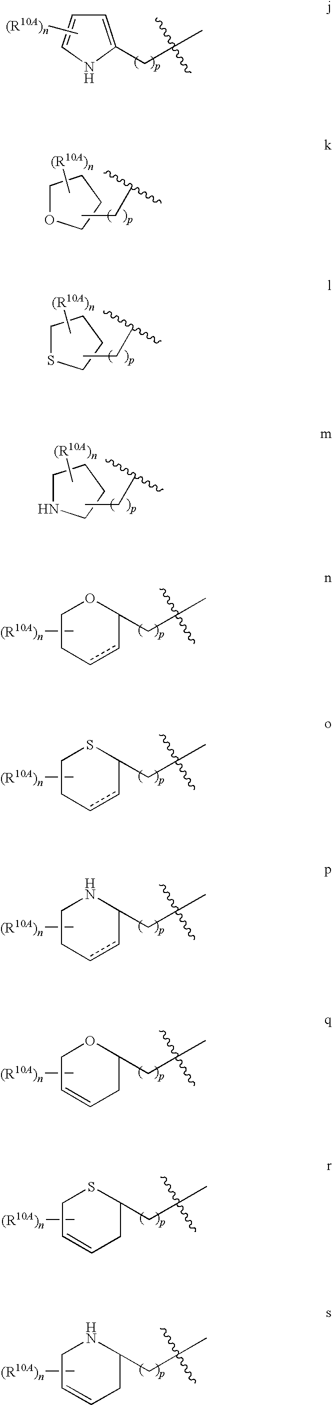

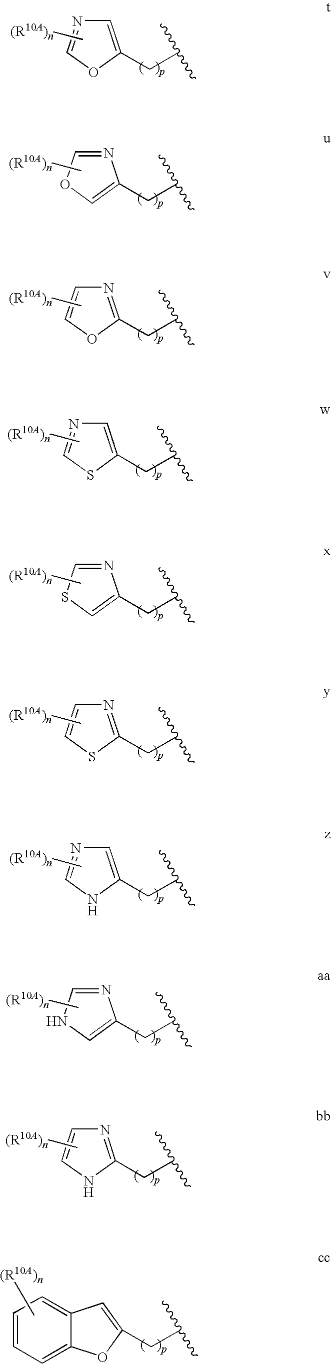

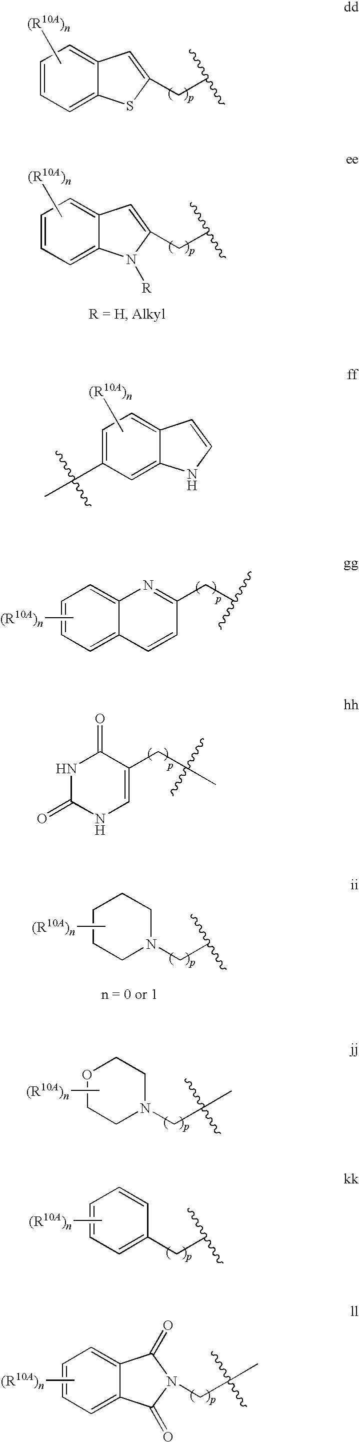

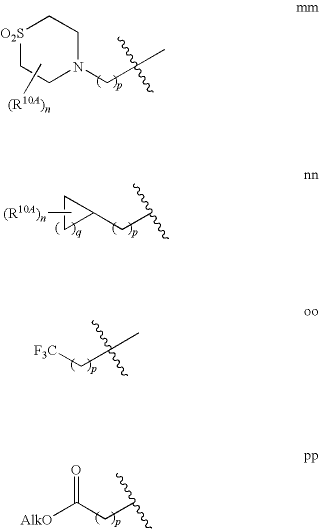

wherein n and p are each independently integers from 0 to 3; q is an integer from 1 to 6; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety;
lxviii) R₁₀ is selected from:

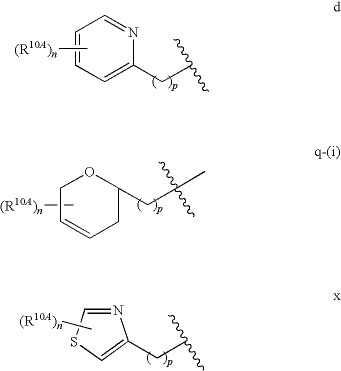

lxix) X₀ is CR^{X0a}R^{X0b}, O or NR^{X0a}; wherein R^{X0a} and R^{X0b} are independently hydrogen, a nitrogen protecting group, or an aliphatic, alicyclic, heteroaliphatic, heteroalicyclic, aryl or heteroaryl moiety;
lxx) X₀ is CR^{X0a}R^{X0b}, O or NR^{X0a}; wherein R^{X0a} and R^{X0b} are independently hydrogen, a nitrogen protecting group, or an alkyl, cycloalkyl, heteroalkyl, heterocyclic, aryl or heteroaryl moiety;
lxxi) X₀ is CR^{X0a}R_{X0b}; wherein R^{X0a} and R^{X0b} are independently hydrogen, a nitrogen protecting group, or an alkyl, cycloalkyl, heteroalkyl, heterocyclic, aryl or heteroaryl moiety;
lxxii) X₀ is CH₂;
lxxiii) X₀ is O;
lxxiv) X₀ is NR^{X0a}; wherein R^{X0a} is hydrogen, a nitrogen protecting group, or an alkyl, cycloalkyl, heteroalkyl, heterocyclic, aryl or heteroaryl moiety;
lxxv) X₁ is O, S or NR^{X1}; wherein R^{X1} is hydrogen, a nitrogen protecting group, or an aliphatic, alicyclic, heteroaliphatic, heteroalicyclic, aryl or heteroaryl moiety;
lxxvi) X₁ is O, S or NR^{X1}; wherein R^{X1} is hydrogen, nitrogen protecting group, or an alkyl, cycloalkyl, heteroalkyl, heterocyclic, aryl or heteroaryl moiety;
lxxvii) X₁ is O;
lxxviii) X₁, taken together with one of R₉ₐ and R_{9b}, forms an alicyclic, heteroalicyclic, aryl or heteroaryl moiety;
lxxix) X₁, taken together with one of R₉ₐ and R_{9b}, forms a substituted or unsubstituted phenyl or pyridinyl moiety;
lxxx) Z is O, NR^{Z1}, CR^{Z1}R^{Z2} or S, wherein R^{Z1} and R^{Z2} are independently hydrogen, halogen, a nitrogen protecting group, or an alkyl, cycloalkyl, heteroalkyl, heterocyclyl, aryl or heteroaryl moiety;
lxxxi) Z is O or NR^{Z1}, wherein R^{Z1} and R^{Z2} are independently hydrogen, halogen, a nitrogen protecting group, or an alkyl, cycloalkyl, heteroalkyl, heterocyclyl, aryl or heteroaryl moiety;
lxxxii) Z is O or NH;
lxxxiii) K, L and M are independently absent, or a substituted or unsubstituted C₁₋₆alkylidene or C₂₋₆alkenylidene chain wherein up to two non-adjacent methylene units are independently optionally replaced by CO, CO₂, COCO, CONR^{P1}, OCONR^{P1}, NR^{P1}NR^{P2}, NR^{P1}NR^{P2}CO, NR^{P1}CO, NR^{P1}CO₂, NR^{P1}CONR^{P2}, SO, SO₂, NR^{P1}SO₂, SO₂NR^{P1}, NR^{P1}SO₂NR^{P2}, O, S, or NR^{P1}; wherein each occurrence of R^{P1} and R^{P2} is independently hydrogen, alkyl, heteroalkyl, aryl, heteroaryl or acyl, or a substitutent present on K, when present, and taken together with R₆, forms a cycloalkyl, heterocyclic, aryl or heteroaryl moiety;
lxxxiv) K, L and M are independently absent, CR^{P1}R^{P2}, CR^{P2} or C═O, wherein R^{P1} is hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety;
lxxxv) K is CR^{P1}R^{P2}, L and M are are connected with a double bond and are independently CR^{P1}; wherein each occurrence of R^{P1} is independently hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety;
lxxxvi) K is CR^{P1}OR^{P2}, L and M are are connected with a double bond and are independently CR^{P1}; wherein each occurrence of R^{P1} is independently hydrogen, halogen, an alkyl, cycloalkyl, heteroalkyl, heterocyclyl, aryl or heteroaryl moiety; R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
lxxxvii) —K-L-M- is a moiety having the structure:

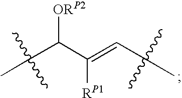

wherein R^{P1} and R^{P2} is as defined in claim 70;
lxxxviii) —K-L-M- has the following stereochemistry:

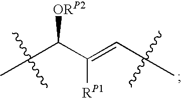

lxxxix) M is CR^{P1}R^{P2}, K and L are are connected with a double bond and are independently CR^{P1}; wherein each occurrence of R^{P1} is independently hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety;
xc) M is CR^{P1}OR^{P2}, K and L are are connected with a double bond and are independently CR^{P1}; wherein each occurrence of R^{P1} is independently hydrogen, halogen, an alkyl, cycloalkyl, heteroalkyl, heterocyclyl, aryl or heteroaryl moiety; R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
xci) —K-L-M- is a moiety having the structure:

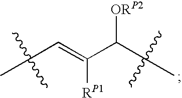

wherein R^{P1} and R^{P2} are as defined in claim 70;
xcii) —K-L-M- has the following stereochemistry:

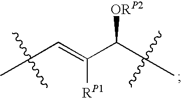

xciii) compounds of subsets lxxv)-lxxvi) and lxxix)-lxxx), wherein R^{P1} is hydrogen or lower alkyl;
xciv) compounds of subsets lxxv)-lxxvi) and lxxix)-lxxx), wherein R^{P1} is hydrogen or methyl;
xcv) K is CR^{P1}R^{P2}, L and M are are connected with a double bond and are independently CR^{P1}; wherein each occurrence of R^{P1} is independently hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety;
xcvi) K is CH₂, L-M together represent a moiety having the structure —C(R^{P1})═C(R^{P1})—; wherein each occurrence of R^{P1} is independently hydrogen, halogen, an alkyl, cycloalkyl, heteroalkyl, heterocyclyl, aryl or heteroaryl moiety;
xcvii) —K-L-M- is a moiety having the structure:

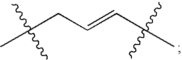

xcviii) —K-L-M-R₁₀ together represent a moiety having the structure:

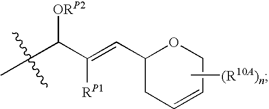

wherein n is an integer from 0 to 6; each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety; R^{P1} is hydrogen, halogen, an alkyl, cycloalkyl, heteroalkyl, heterocyclyl, aryl or heteroaryl moiety; and R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
xcix) —K-L-M-R₁₀ together represent a moiety having the structure:

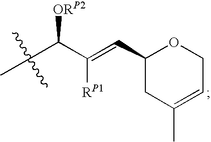

wherein R^{P1} is hydrogen or lower alkyl;
c) —K-L-M-R₁₀ together represent a moiety having the structure:

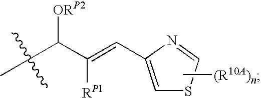

wherein n is an integer from 0 to 2; each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety; R^{P1} is independently hydrogen, halogen, an alkyl, cycloalkyl, heteroalkyl, heterocyclyl, aryl or heteroaryl moiety; and R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
ci) —K-L-M-R₁₀ together represent a moiety having the structure:

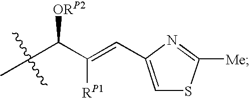

wherein R^{P1} is hydrogen or lower alkyl;
cii) —K-L-M-R₁₀ together represent a moiety having the structure:

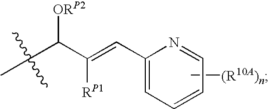

wherein n is an integer from 0 to 4; each occurrence of R^{10A} is independently hydrogen, halogen, —CN, or WR^{W1} wherein W is O, S, NR^{W2}; —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group; a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety; R^{P1} is independently hydrogen, halogen, an alkyl, cycloalkyl, heteroalkyl, heterocyclyl, aryl or heteroaryl moiety; and R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
ciii) —K-L-M-R₁₀ together represent a moiety having the structure:

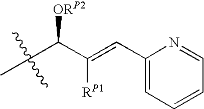

wherein R^{P1} is hydrogen or lower alkyl;
civ) —K-L-M-R₁₀ together represent a moiety having the structure:

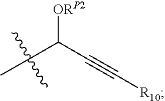

wherein R₁₀ is hydrogen or an alkyl, cycloalkyl, heteroalkyl, heterocyclic, aryl or heteroaryl moiety; and R^{P2} is hydrogen, a protecting group, a prodrug moiety, —C(═O)R^{y}, or an alkyl, cycloalkyl, heteroalkyl, heterocyclyl, aryl or heteroaryl moiety; wherein R^{y} is hydrogen, or an alkyl, cycloalkyl, heteroalkyl, heterocyclic, aryl or heteroaryl moiety;
cv) compounds of subset xcii) wherein R₁₀ is a cycloalkyl, heterocyclic, aryl or heteroaryl moiety;
cvi) —K-L-M-R₁₀ has the following stereochemistry:

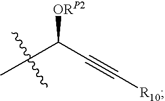

cvii) compounds of subsets xcii) and xciv), wherein R₁₀ is one of a through pp, as described above;
cviii) A, B, D, E, G and J are independently connected by either a single or double bond, as valency permits, or -(A)_{q}-B-D-E-(G)ₜ-J- or A-B-D-E-G-J together represents an aryl or heteroaryl moiety; wherein B and J are independently N or CR^{Q1}; and A, D, E and G are independently C═O, CR^{Q1}R^{Q2}, NR^{Q1}, N, O or S; wherein each occurrence of R^{Q1} and R^{Q2} is independently absent, hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; or any two adjacent substituents on A, B, D, E, G and J, taken together, may represent a cycloalkyl, heterocyclic, aryl or heteroaryl moiety;
cix) -(A)_{q}-B-D-E-(G)ₜ-J- or A-B-D-E-G-J together represent a heterocyclic moiety having the structure:

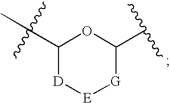

wherein at least one of D and E, and E and G are connected by a double bond; and D, E and G are independently C═O, CR^{Q1}R^{Q2}, NR^{Q1}, N, O or S; wherein each occurrence of R^{Q1} and R^{Q2} is independently absent, hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; or any two adjacent substituents on D, E and G, taken together, may represent a cycloalkyl, heterocyclic, aryl or heteroaryl moiety;
cx) compounds of subset xcvii) above, wherein the heterocyclic moiety has the following stereochemistry:

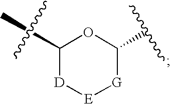

cxi) -(A)_{q}-B-D-E-(G)ₜ-J- or A-B-D-E-G-J together represent a heterocyclic moiety having the structure:

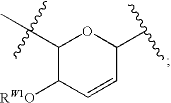

wherein R^{W1} is hydrogen, a protecting group, a prodrug moiety, —C(═O)R^{y3}, or an alkyl, cycloalkyl, heteroalkyl, heterocyclic, aryl or heteroaryl moiety; wherein R^{y3} is hydrogen, or an alkyl, cycloalkyl, heteroalkyl, heterocyclic, aryl or heteroaryl moiety;
cxii) compounds of subset xcix) above, wherein the heterocyclic moiety has the following stereochemistry:

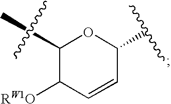

cxiii) -(A)_{q}-B-D-E-(G)ₜ-J- or A-B-D-E-G-J together represent a heterocyclic moiety having the structure:

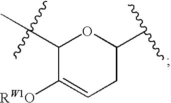

wherein R^{W1} is hydrogen, a protecting group, a prodrug moiety, —C(═O)R^{y3}, or an alkyl, cycloalkyl, heteroalkyl, heterocyclic, aryl or heteroaryl moiety; wherein R^{y3} is hydrogen, or an alkyl, cycloalkyl, heteroalkyl, heterocyclic, aryl or heteroaryl moiety;
cxiv) compounds of subset xci) above, wherein the heterocyclic moiety has the following stereochemistry:

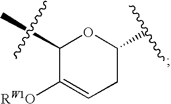

cxv) -(A)_{q}-B-D-E-(G)ₜ-J- or A-B-D-E-G-J together represent a heterocyclic moiety having the structure:

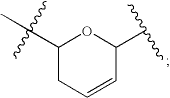

compounds of subset ciii) above, wherein the heterocyclic moiety has the following stereochemistry:

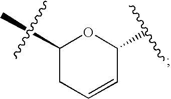

cxvi) -(A)_{q}-B-D-E-(G)ₜ-J- or A-B-D-E-G-J together represent a heterocyclic moiety having the structure:

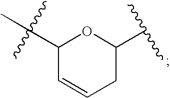

wherein R^{W1} is hydrogen, a protecting group, a prodrug moiety, —C(═O)R^{y3}, or an alkyl, cycloalkyl, heteroalkyl, heterocyclic, aryl or heteroaryl moiety; wherein R^{y3} is hydrogen, or an alkyl, cycloalkyl, heteroalkyl, heterocyclic, aryl or heteroaryl moiety;
cxvii) compounds of subset cv) above, wherein the heterocyclic moiety has the following stereochemistry:

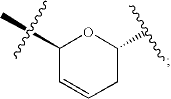

cxviii) compounds of subsets xcix) through cii) above, wherein R^{W1} is hydrogen, an oxygen protecting group or lower alkyl;
cxix) compounds of subset cvii) above, wherein R^{W1} is methyl;
cxx) -(A)_{q}-B-D-E-(G)ₜJ- or A-B-D-E-G-J together represent a heterocyclic moiety having the structure:

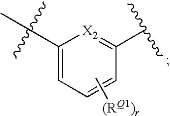

wherein X₂ is CH or N; r is an integer from 0 to 3; and each occurrence of R^{Q1} is independently hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety;
cxxi) q and t are each 1;
cxxii) q is 1 and t is 2;
cxxiii) q is 2 and t is 1;
cxxiv) q+t is 1;
cxxv) n is 0;
cxxvi) n is 1 and R^{10A} is lower alkyl;
cxxvii) n is 1 and R^{10A} is methyl;
cxxviii) r is 0;
cxxix) r is 1 and R^{Q1} is lower alkyl or OR^{W}1 wherein R^{W1} is hydrogen or lower alkyl; and/or
cxxx) r is 1 and R^{Q1} is OMe.

It will be appreciated that for each of the classes and subclasses described above and herein, any one or more occurrences of aliphatic, heteroaliphatic, alkyl, heteroalkyl may independently be substituted or unsubstituted, cyclic or acyclic, linear or branched and any one or more occurrences of aryl, heteroaryl, alicyclic, heteroalicyclic may be substituted or unsubstituted.

The reader will also appreciate that all possible combination of the variables described in i)-through cxxx) above (e.g., R₁-R₁₀, q, t, X₀, X₁, A, B, D, E, G, J, K, L, M and Z, among others) is considered part of the invention. Thus, the invention encompasses any and all compounds of formula I generated by taking any possible permutation of the variables described in i)-through cxxx) above.

As the reader will appreciate, compounds of particular interest include, among others, those which share the attributes of one or more of the foregoing subclasses. Some of those subclasses are illustrated by the following sorts of compounds:

I) Compounds Having the Structure (and Pharmaceutically Acceptable Derivatives Thereof):

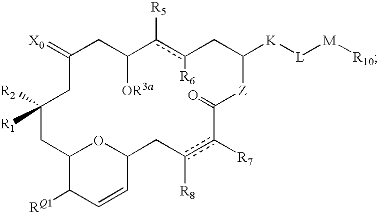

wherein R₁, R₂, R₅, R₆, R₇, R₈, R₁₀, X₀, Z, K, L and M are as defined generally above and in classes and subclasses herein; e^{l} is hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; and R^{3a} is hydrogen, an oxygen protecting group, a prodrug moiety or an alkyl, cycloalkyl, heteroalkyl, heterocyclic, aryl or heteroaryl moiety.

In certain embodiments, X₀ is CH₂ and the compound has the structure:

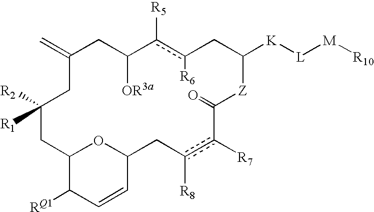

In certain embodiments, R₅ and R₆ and the carbon atoms to which they are attached form a 3-membered cyclic moiety; and the compound has the structure:

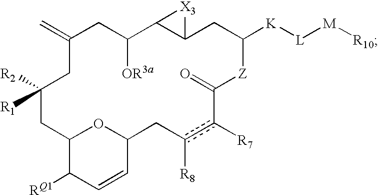

wherein X₃ is CR^{X3a}R^{X3b}, O or NR^{X3a}; wherein R^{X3a} and R^{X3b} are independently hydrogen, a nitrogen protecting group, or an alkyl, cycloalkyl, heteroalkyl, heterocyclic, aryl or heteroaryl moiety.

In certain other embodiments, the carbon atoms to which R₇ and R₈ are attached are connected with a single bond; and the compound has the structure:

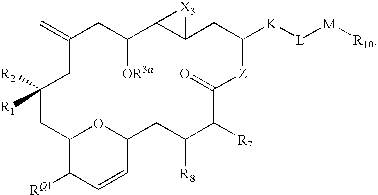

In certain other embodiments, the carbon atoms to which R₇ and R₈ are attached are connected with a cis-double bond; and the compound has the structure:

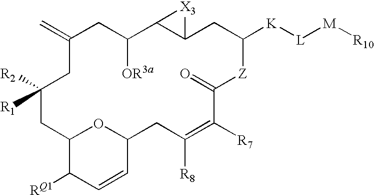

In certain other embodiments, the carbon atoms to which R₇ and R₈ are attached are connected with a trans-double bond; and the compound has the structure:

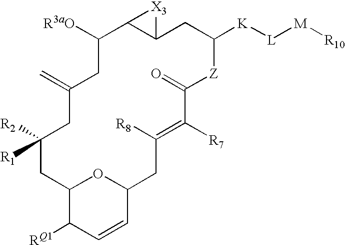

In certain other embodiments, R₇ and R₈ are absent; the carbon atoms to which R₇ and R₈ are attached are connected with a triple bond; and the compound has the structure:

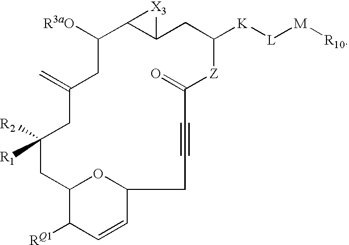

II) Compounds Having the Structure (and Pharmaceutically Acceptable Derivatives Thereof):

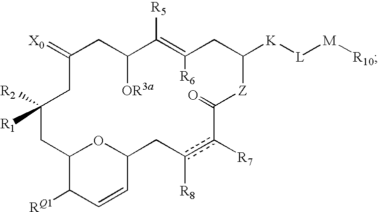

wherein R₁, R₂, R₅, R₆, R₇, R₈, R₁₀, X₀, Z, K, L and M are as defined generally above and in classes and subclasses herein; R^{Q1} is hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; and R^{3a} is hydrogen, an oxygen protecting group, a prodrug moiety or an alkyl, cycloalkyl, heteroalkyl, heterocyclic, aryl or heteroaryl moiety.

In certain embodiments, X₀ is CH₂ and the compound has the structure:

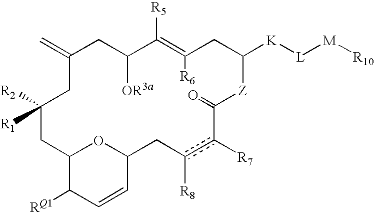

In certain other embodiments, the carbon atoms to which R₇ and R₈ are attached are connected with a single bond; and the compound has the structure:

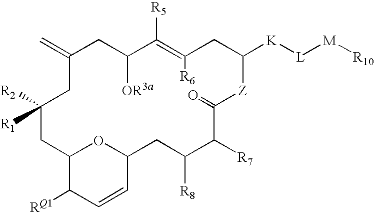

In certain other embodiments, the carbon atoms to which R₇ and R₈ are attached are connected with a cis-double bond; and the compound has the structure:

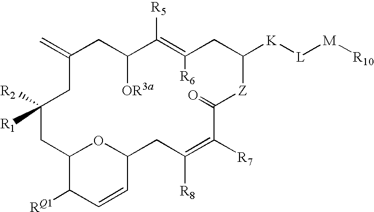

In certain other embodiments, the carbon atoms to which R₇ and R₈ are attached are connected with a trans-double bond; and the compound has the structure:

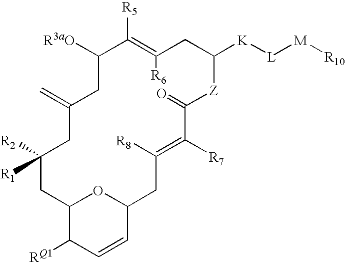

In certain other embodiments, R₇ and R₈ are absent; the carbon atoms to which R₇ and R₈ are attached are connected with a triple bond; and the compound has the structure:

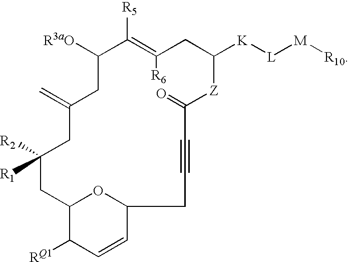

III) Compounds Having the Structure and Pharmaceutically Acceptable Derivatives Thereof):

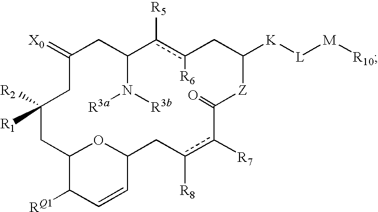

wherein R₁, R₂, R₅, R₆, R₇, R₈, R₁₀, X₀, Z, K, L and M are as defined generally above and in classes and subclasses herein; R^{Q1} is hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; and R^{3a} and R^{3b} are independently hydrogen, a nitrogen protecting group, a prodrug moiety or an alkyl, cycloalkyl, heteroalkyl, heterocyclic, acyl, aryl or heteroaryl moiety. In certain embodiments, R^{3a} and R^{3b} are independently hydrogen, lower alkyl or acyl.

In certain embodiments, X₀ is CH₂ and the compound has the structure:

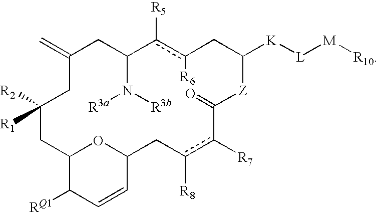

In certain embodiments, R₅ and R₆ and the carbon atoms to which they are attached faun a 3-membered cyclic moiety having the structure:

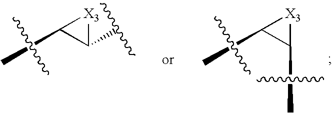

wherein X₃ is CR^{X3a}R^{X3b}, O or NR^{X3a}; wherein R^{X3a} and R^{X3b} are independently hydrogen, a nitrogen protecting group, or an alkyl, cycloalkyl, heteroalkyl, heterocyclic, acyl, aryl or heteroaryl moiety. In certain embodiments, X₃ is CH₂, O or NR^{X3a}; wherein R^{X3a} is hydrogen, a nitrogen protecting group, or an alkyl, cycloalkyl, heteroalkyl, heterocyclic, acyl, aryl or heteroaryl moiety. In certain exemplary embodiments, X₃ is CH₂ or O.

In certain other embodiments, the carbon atoms to which R₇ and R₈ are attached are connected with a single bond, a cis-double bond, a trans-double bond, or a triple bond.

IV) Compounds Having the Structure (and Pharmaceutically Acceptable Derivatives Thereof):

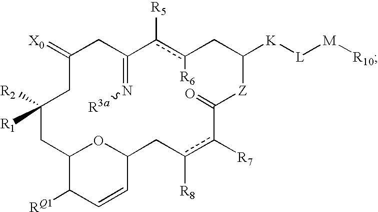

wherein R₁, R₂, R₅, R₆, R₇, R₈, R₁₀, X₀, Z, K, L and M are as defined generally above and in classes and subclasses herein; R^{Q1} is hydrogen, halogen, an alkyl, cycloalkyl, heteroalkyl, heterocyclyl, aryl or heteroaryl moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclyl, aryl or heteroaryl moiety, or, when W is NR^{W2}, R^{W1} and R^{W2}, taken together with the nitrogen atom to which they are attached, form a heterocyclic or heteroaryl moiety; and R^{3a} is hydrogen, a nitrogen protecting group, a prodrug moiety, an alkyl, cycloalkyl, heteroalkyl, heterocyclic, acyl, aryl or heteroaryl moiety; or OR^{3b} wherein R^{3b} is hydrogen, or an alkyl, cycloalkyl, heteroalkyl, heterocyclic, aryl or heteroaryl moiety. In certain embodiments, R^{3a} is OH or OR³b wherein R^{3b} is lower alkyl. In certain exemplary embodiments, R^{3a} is OMe.

In certain embodiments, X₀ is CH₂ and the compound has the structure:

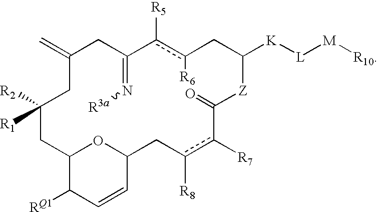

In certain embodiments, R₅ and R₆ and the carbon atoms to which they are attached form a 3-membered cyclic moiety having the structure:

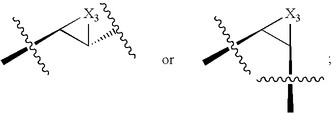

wherein X₃ is CR^{X3a}R^{X3b}, O or NR^{X3a}; wherein R^{X3a} and R^{X3b} are independently hydrogen, a nitrogen protecting group, or an alkyl, cycloalkyl, heteroalkyl, heterocyclic, acyl, aryl or heteroaryl moiety. In certain embodiments, X₃ is CH₂, O or NR^{X3a}; wherein R^{X3a} is hydrogen, a nitrogen protecting group, or an alkyl, cycloalkyl, heteroalkyl, heterocyclic, acyl, aryl or heteroaryl moiety. In certain exemplary embodiments, X₃ is CH₂ or O.

In certain other embodiments, R₇ and R₈ are attached are connected with a single bond, a cis-double bond, a trans-double bond, or a triple bond.

V) Compounds Having the Structure (and Pharmaceutically Acceptable Derivatives Thereof):

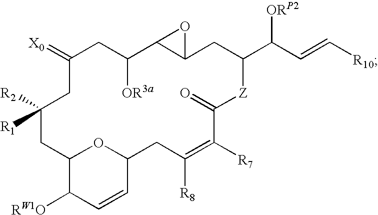

wherein X₀ is as defined generally above and in classes and subclasses herein; Z is O, NH or NR^{Z1}, wherein R^{Z1} is a nitrogen protecting group, alkyl, aryl or heteroaryl; R₁ and R₂ are independently hydrogen or lower alkyl; R^{3a}, R^{W1} and R^{P2} are independently hydrogen, an oxygen protecting group, a prodrug moiety, lower alkyl, aryl or heteroaryl; R₇ and R₈ are independently hydrogen, halogen, lower alkyl, aryl, heteroaryl, or, R₇ and R₈, taken together, form a cycloalkyl, heterocyclyl, aryl or heteroaryl moiety.

In certain embodiments, X₀ is CH₂ and the compound has the structure:

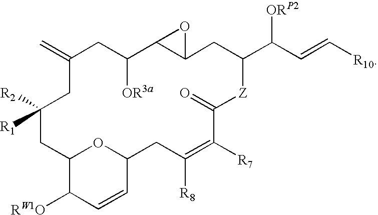

In certain embodiments, compounds have the following stereochemistry:

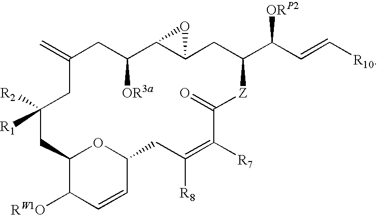

VI) Compounds Having the Structure (and Pharmaceutically Acceptable Derivatives Thereof):

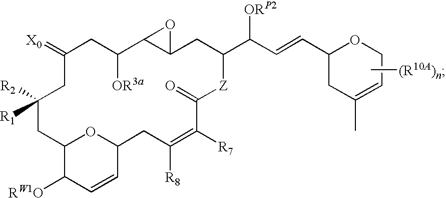

wherein X₀ is as defined generally above and in classes and subclasses herein; Z is O, NH or NR^{Z1}, wherein R^{Z1} is a nitrogen protecting group, alkyl, aryl or heteroaryl; R₁ and R₂ are independently hydrogen or lower alkyl; R^{3a}, R^{W1} and R^{P2} are independently hydrogen, an oxygen protecting group, a prodrug moiety, lower alkyl, aryl or heteroaryl; R₇ and R₈ are independently hydrogen, halogen, lower alkyl, aryl, heteroaryl, or, R₇ and R₈, taken together, form a cycloalkyl, heterocyclyl, aryl or heteroaryl moiety.

In certain embodiments, X₀ is CH₂ and the compound has the structure:

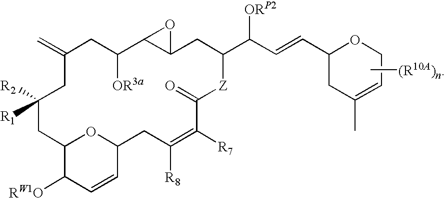

In certain embodiments, compounds have the following stereochemistry:

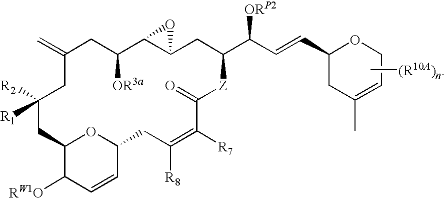

VII) Compounds Having the Structure (and Pharmaceutically Acceptable Derivatives Thereof):

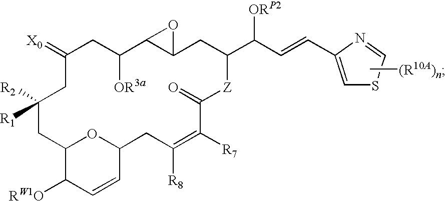

wherein X₀ is as defined generally above and in classes and subclasses herein; n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety.

In certain embodiments, X₀ is CH₂ and the compound has the structure:

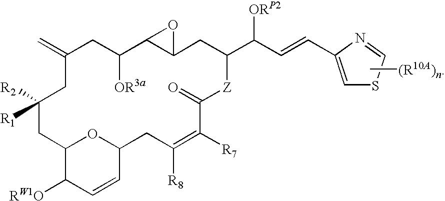

In certain embodiments, compounds have the following stereochemistry:

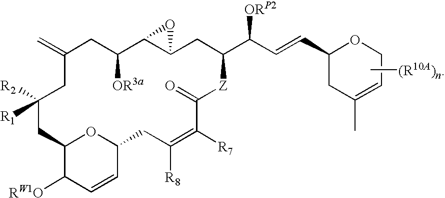

VIII) Compounds Having the Structure (and Pharmaceutically Acceptable Derivatives Thereof):

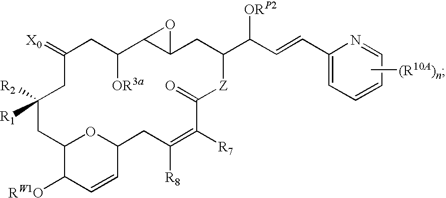

wherein X₀ is as defined generally above and in classes and subclasses herein; n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety.

In certain embodiments, X₀ is CH₂ and the compound has the structure:

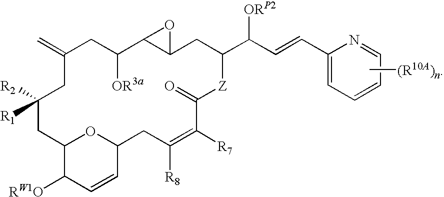

In certain embodiments, compounds have the following stereochemistry:

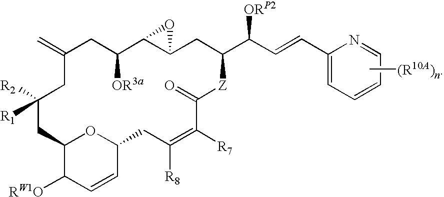

IX) Compounds Having the Structure (and Pharmaceutically Acceptable Derivatives Thereof):

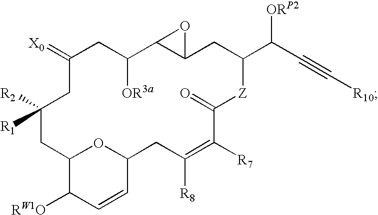

wherein R₁, R₂, R₇, R₈, R₁₀, X₀ and Z are as defined generally above and in classes and subclasses herein; and R^{3a}, R^{P2} and R^{W1} are independently hydrogen, a protecting group, a prodrug moiety or an alkyl, cycloalkyl, heteroalkyl, heterocyclic, aryl or heteroaryl moiety.

In certain embodiments, X₀ is CH₂ and the compound has the structure:

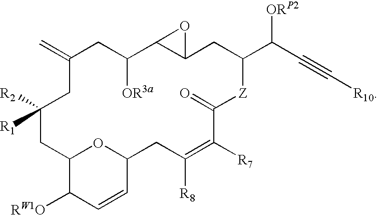

In certain embodiments, Z is O, NH or NR^{Z1}, wherein R^{Z1} is a nitrogen protecting group, alkyl, aryl or heteroaryl; R₁ and R₂ are independently hydrogen or lower alkyl; R^{3a}, R^{W1} and R^{P2} are independently hydrogen, an oxygen protecting group, a prodrug moiety, lower alkyl, aryl or heteroaryl; R₇ and R₈ are independently hydrogen, halogen, lower alkyl, aryl or heteroaryl. In certain exemplary embodiment, R₇ and R₈ are each hydrogen.

In certain embodiments, compounds have the following stereochemistry:

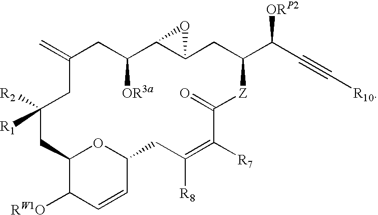

X) Compounds Having the Structure (and Pharmaceutically Acceptable Derivatives Thereof):

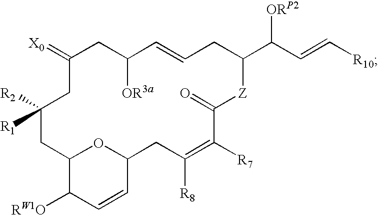

wherein X₀ is as defined generally above and in classes and subclasses herein; Z is O, NH or NR^{Z1}, wherein R^{Z1} is a nitrogen protecting group, alkyl, aryl or heteroaryl; R₁ and R₂ are independently hydrogen or lower alkyl; R^{3a}, R^{W1} and R^{P2} are independently hydrogen, an oxygen protecting group, a prodrug moiety, lower alkyl, aryl or heteroaryl; R₇ and R₈ are independently hydrogen, halogen, lower alkyl, aryl, heteroaryl, or, R₇ and R₈, taken together, form a cycloalkyl, heterocyclyl, aryl or heteroaryl moiety.

In certain embodiments, X₀ is CH₂ and the compound has the structure:

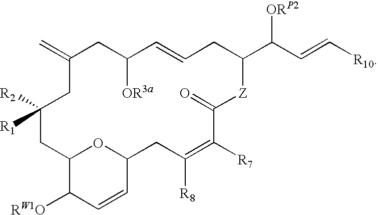

In certain embodiments, compounds have the following stereochemistry:

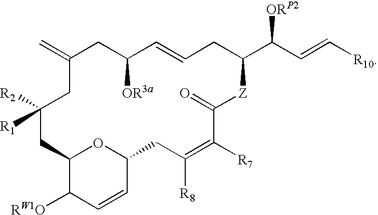

XI) Compounds Having the Structure (and Pharmaceutically Acceptable Derivatives Thereof):

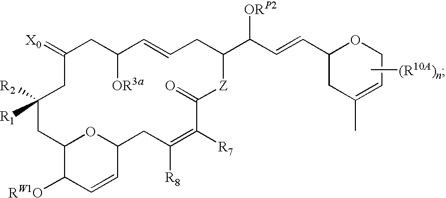

wherein X₀ is as defined generally above and in classes and subclasses herein; n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety.

In certain embodiments, X₀ is CH₂ and the compound has the structure:

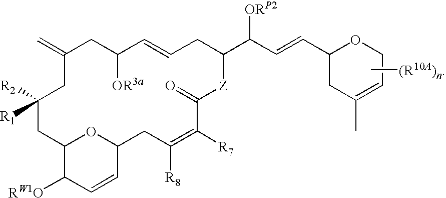

In certain embodiments, compounds have the following stereochemistry:

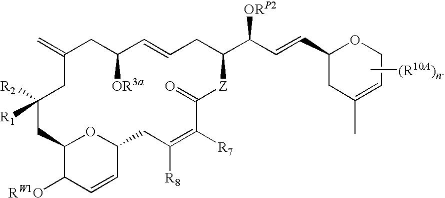

XII) Compounds Having the Structure (and Pharmaceutically Acceptable Derivatives Thereof):

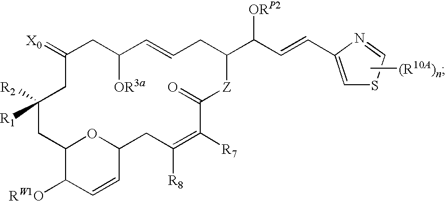

wherein X₀ is as defined generally above and in classes and subclasses herein; n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen,a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety.

In certain embodiments, X₀ is CH₂ and the compound has the structure:

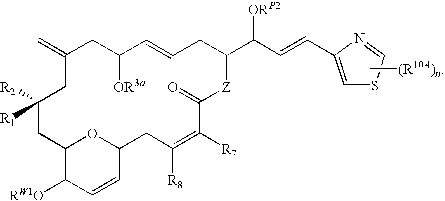

In certain embodiments, compounds have the following stereochemistry:

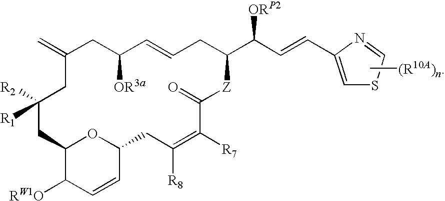

XIII) Compounds Having the Structure (and Pharmaceutically Acceptable Derivatives Thereof):

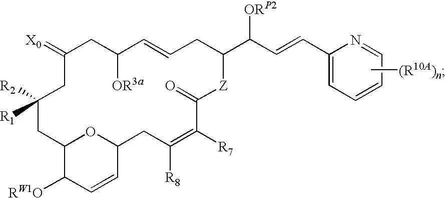

wherein X₀ is as defined generally above and in classes and subclasses herein; n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is R^{W2}, R^{W1} and R^{W2}, taken together with the nitrogen atom to which they are attached, form a heterocyclic or heteroaryl moiety.

In certain embodiments, X₀ is CH₂ and the compound has the structure:

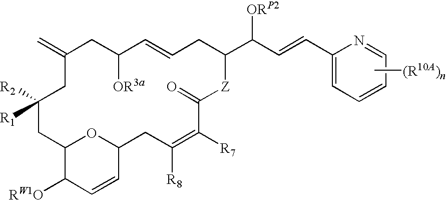

In certain embodiments, compounds have the following stereochemistry:

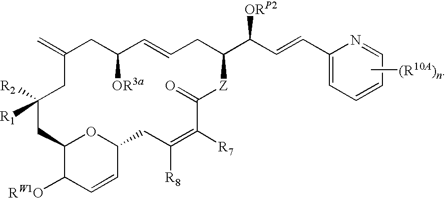

XIV) Compounds Having the Structure (and Pharmaceutically Acceptable Derivatives Thereof):

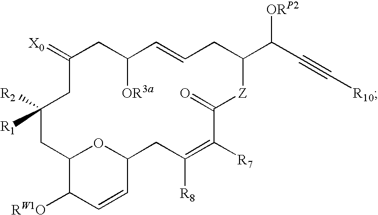

wherein R₁, R₂, R₇, R₈, R₁₀, X₀ and Z are as defined generally above and in classes and subclasses herein; and R^{3a}, R^{P2} and R^{W1} are independently hydrogen, a protecting group, a prodrug moiety or an alkyl, cycloalkyl, heteroalkyl, heterocyclic, aryl or heteroaryl moiety.

In certain embodiments, X₀ is CH₂ and the compound has the structure:

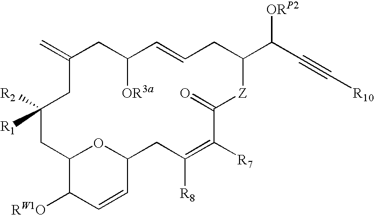

In certain embodiments, Z is O, NH or NR^{Z1}, wherein R^{Z1} is a nitrogen protecting group, alkyl, aryl or heteroaryl; R₁ and R₂ are independently hydrogen or lower alkyl; R^{3a}, R^{W1} and R^{P2} are independently hydrogen, an oxygen protecting group, a prodrug moiety, lower alkyl, aryl or heteroaryl; R₇ and R₈ are independently hydrogen, halogen, lower alkyl, aryl or heteroaryl. In certain exemplary embodiment, R₇ and R₈ are each hydrogen.

In certain embodiments, compounds have the following stereochemistry:

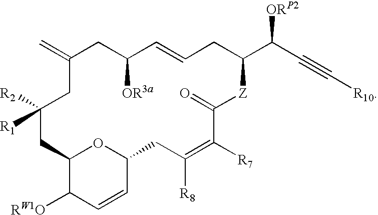

XV) Compounds Having the Structure (and Pharmaceutically Acceptable Derivatives Thereof):

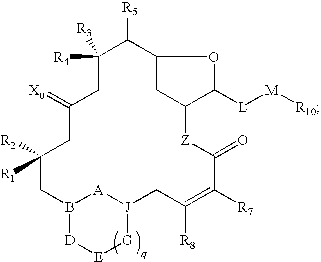

wherein q, R₁-R₅, R₇-R₈, R₁₀, X₀, A, B, D, E, G, J, L, M and Z are as defined generally above and in classes and subclasses herein.

In certain embodiments, X₀ is CH₂ and the compound has the structure:

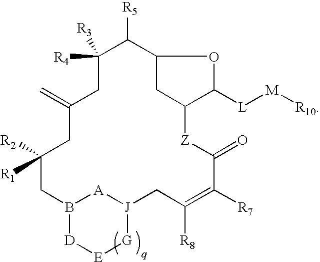

In certain embodiments, Z is O, R₅, R₇ and R₈ are each hydrogen, R₃ and R₄ together represent a carbonyl, and the compound has the structure:

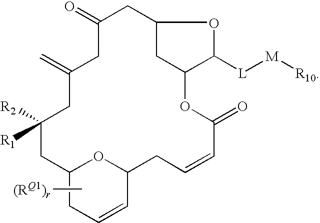

In certain embodiments, the compound has the following stereochemistry:

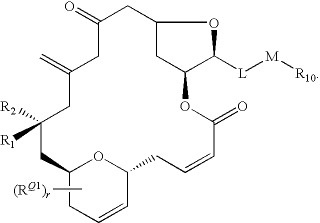

In certain embodiments, -L-M-R¹⁰ is one of:

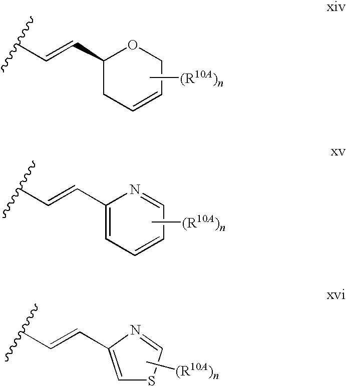

wherein n is an integer from 0 to 3; and each occurrence of R^{10A} is independently hydrogen, halogen, —CN, or WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an alkyl, cycloalkyl, heteroalkyl, heterocyclic, aryl or heteroaryl moiety, or, when W is NR^{W2}, R^{W1} and R^{W2}, taken together with the nitrogen atom to which they are attached, form a heterocyclic or heteroaryl moiety.

In certain exemplary embodiments, -L-M-R¹⁰ is xiv and the compound has the structure:

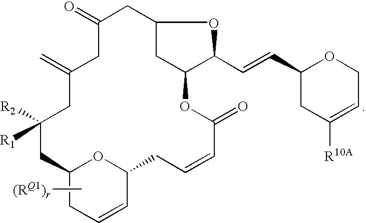

In certain exemplary embodiments, r is 0, R₁ and R^{10A} are each methyl and R₂ is hydrogen.

In certain embodiments, for compounds of classes I-XVI above, R₁ is methyl and R₂ is hydrogen. In certain other embodiments, R₁ and R₂ are each methyl. In certain other embodiments, R^{3a} is hydrogen, methyl or acetyl. In certain other embodiments, R^{P2} is hydrogen, methyl or acetyl. In certain other embodiments, R₇ and R₈ are each hydrogen. In certain other embodiments, R^{W1} is hydrogen or methyl. In certain other embodiments, Z is O or NR^{Z1} wherein R^{Z1} is hydrogen, lower alkyl or aryl.

In certain embodiments, any one or more of the following structures is part of the invention:

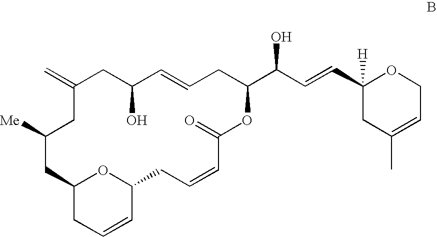

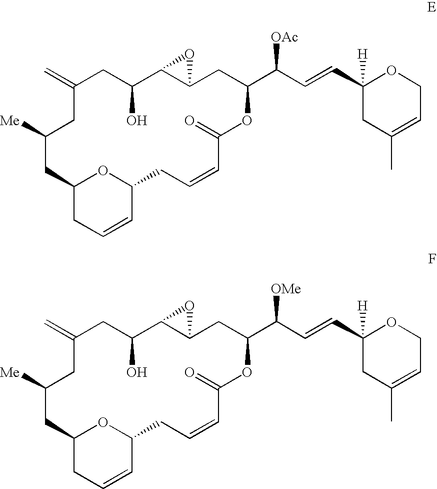

It will be appreciated that each of the compounds described herein and each of the subclasses of compounds described above (I-XV) may be substituted as described generally herein, or may be substituted according to any one or more of the subclasses described above and herein (e.g., i-cxxx).

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of compound of formula (I) under different conditions and may exist as one or a combination of polymorphs of compound of general formula (I) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts, their pharmaceutically acceptable solvates, and pharmaceutically acceptable compositions containing them.

As discussed above, this invention provides novel compounds with a range of biological properties. Preferred compounds of this invention have biological activities relevant for the treatment of cancer and disorders associated with cell hyperproliferation.

Compounds of this invention include those specifically set forth above and described herein, and are illustrated in part by the various classes, subgenera and species disclosed elsewhere herein.

2) Synthetic Methodology

The practitioner has a a well-established literature of macrolide chemistry to draw upon, in combination with the information contained herein, for guidance on synthetic strategies, protecting groups, and other materials and methods useful for the synthesis of the compounds of this invention, including compounds containing the various R₁-R₁₀, q, t, X₀, X₁, A, B, D, E, G, J, K, L, M and Z substituents.

The various patent documents and other references cited herein provide helpful background information on preparing compounds similar to the inventive compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

As described above, the present invention provides novel compounds, specifically compounds having the following general structure:

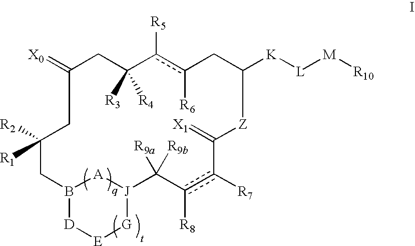

and pharmaceutically acceptable derivatives thereof;

wherein R₁-R₁₀, q, t, X₀, X₁, A, B, D, E, G, J, K, L, M and Z are defined in classes and subclasses herein.

In yet another aspect of the invention, methods for producing intermediates useful for the preparation of compounds of formulae (I) are provided, embodiments of said methods being depicted generally in Schemes 1-14 below.

For example, acetonide 5 may be prepared by coupling of bromide 1 with enal 2a, followed by protection of the resulting hydroxyl group to give acetonide 3 (Scheme 1). Reaction with the free hydroxyl group of intermediate 3 with an appropriate phosphonate reagent, followed by TBS deprotection and oxidation (e.g., Dess-Martin) affords intermediate 4, which may be cyclized under suitable conditions to yield macrocylic acetonide intermediate 5.

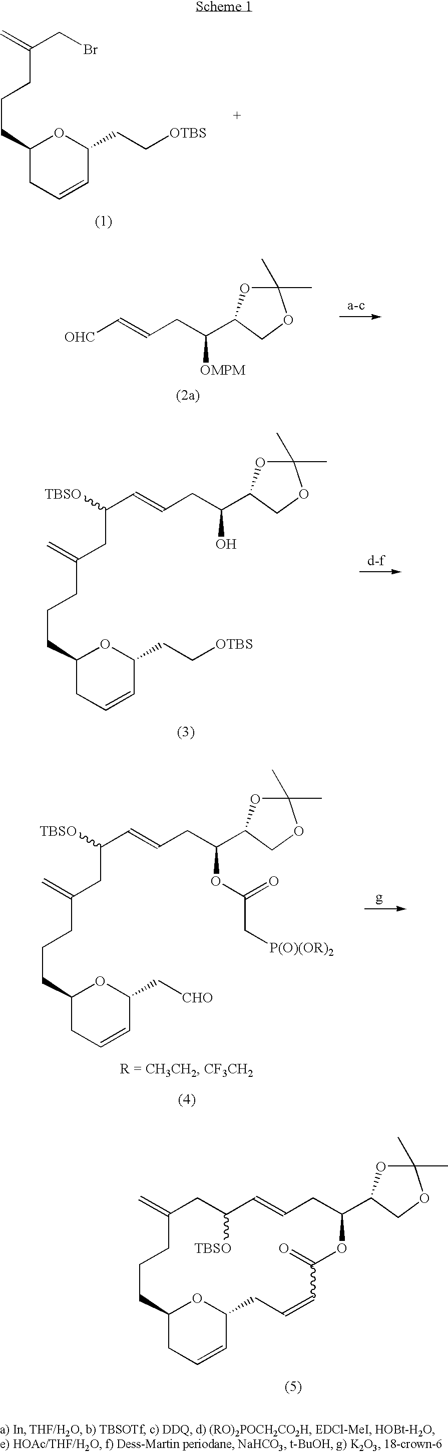

Acetonide 5 may be further functionalized in a variety of ways to give the —K-L-M-R₁₀ side chain (see formula I) of interest. For example, acetonide 5 may be hydrolyzed to the corresponding diol 6, which may be further functionalized by attachment of suitable side chain fragments on one or both the side chain hydroxyl groups.

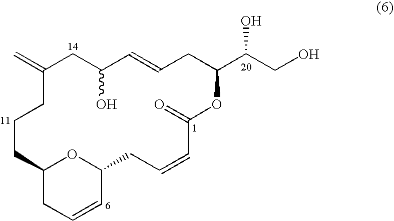

In certain embodiments, 20-epi-11-methyl acetonide 12 may be prepared from bromide 7 and enal 2b according to Scheme 2.

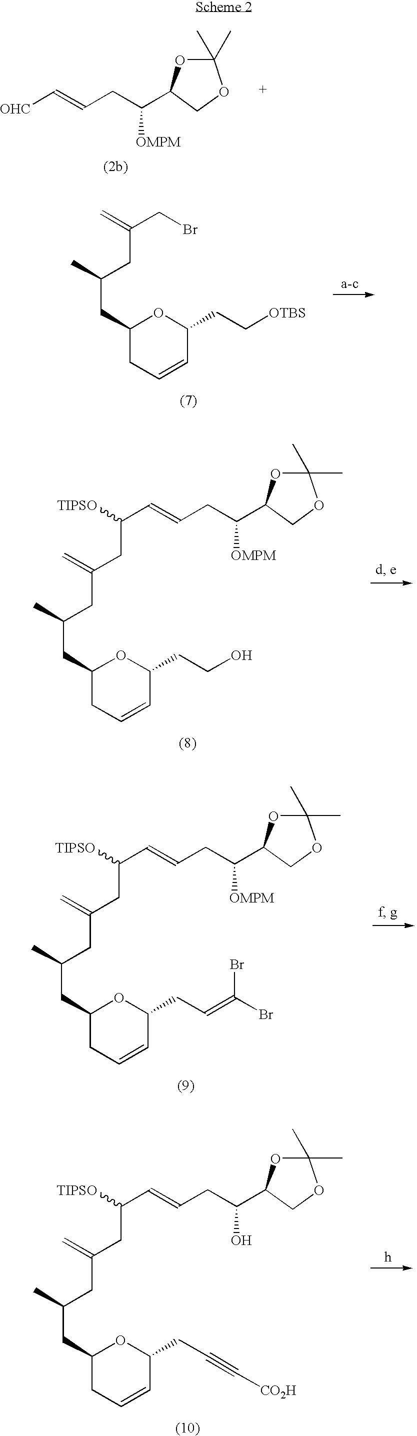

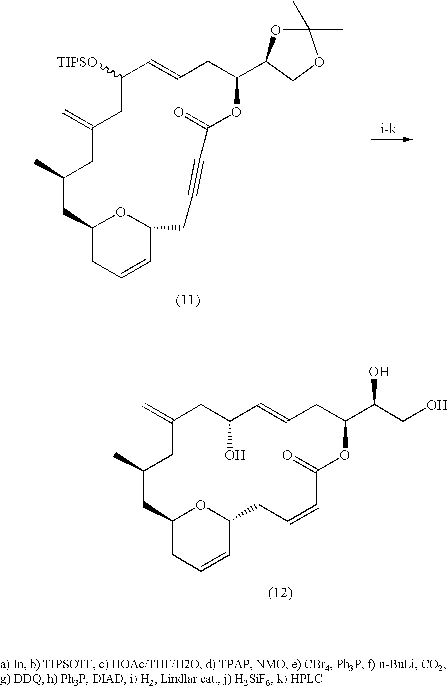

In certain embodiments, enal 2a may be obtained through the methodology depicted in Scheme 3. In certain embodiments, enal 2b may be obtained through a similar method, starting from a different saccharide.

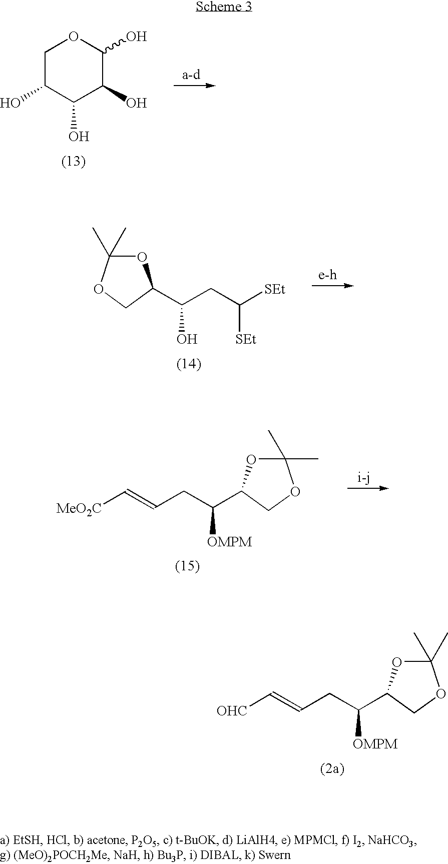

In certain embodiments, bromide 1 may be obtained through the methodology depicted in Scheme 4.

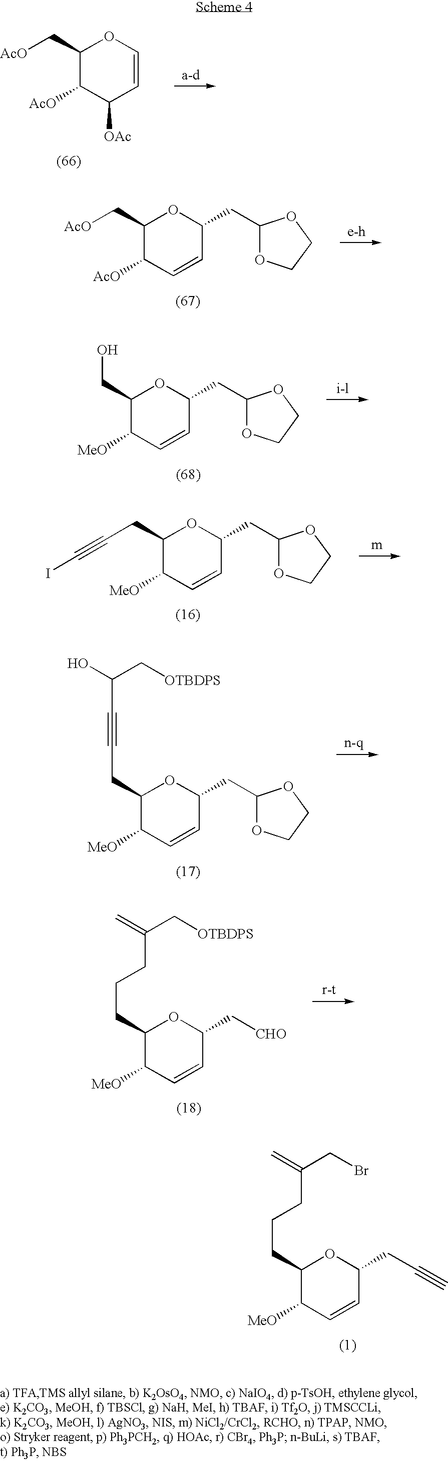

In certain embodiments, bromide 7 may be obtained through the methodology depicted in Scheme 5.

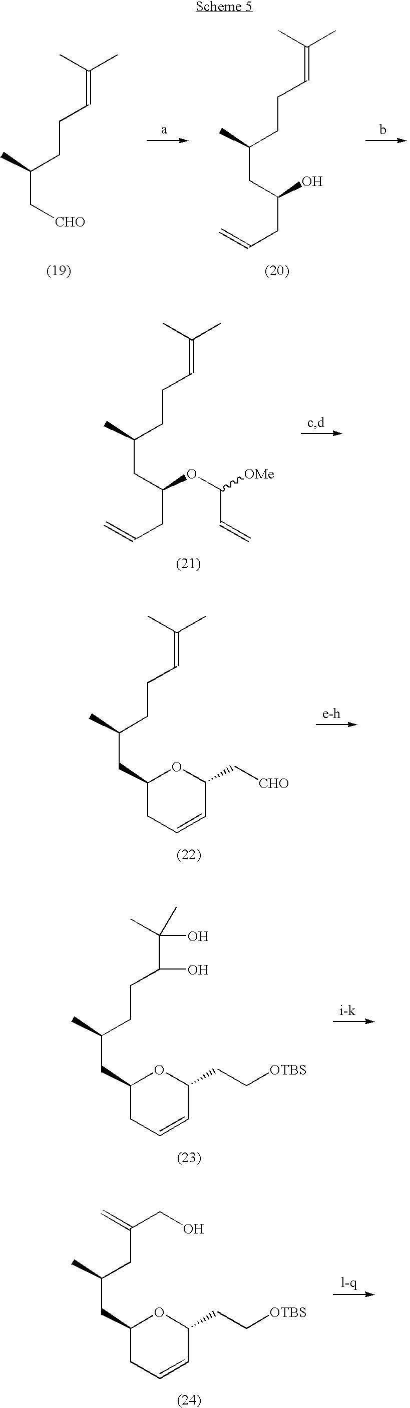

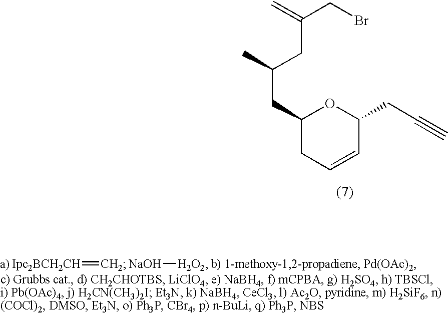

In certain other exemplary embodiments, bromide 7 may be obtained through the methodology depicted in Scheme 6.

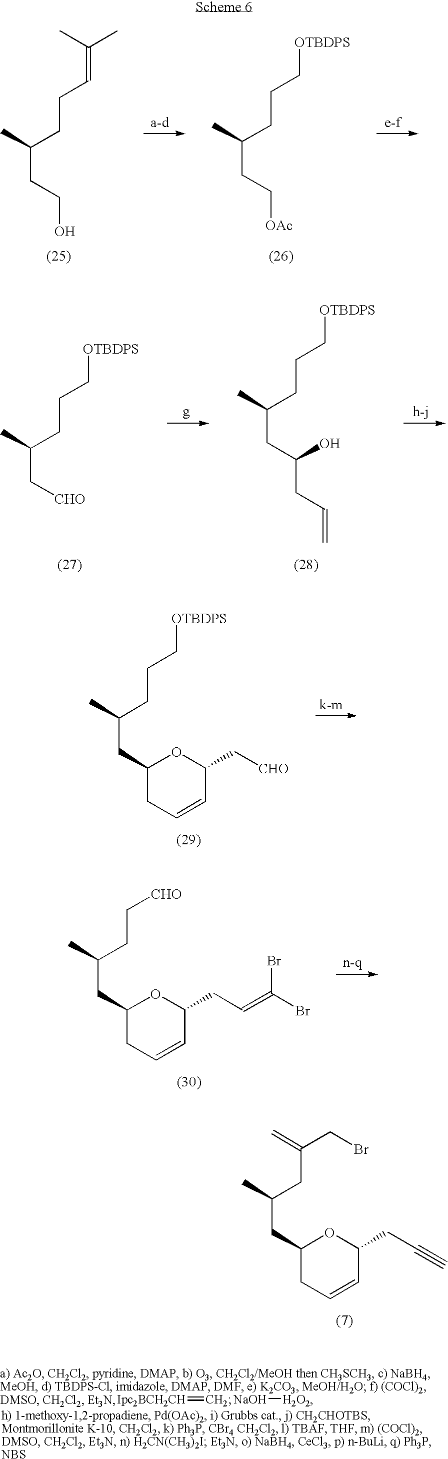

In certain other embodiments, compounds of formula I wherein —K-L-M-R₁₀ is a moiety having the structure:

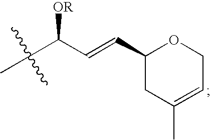

may be obtained by using intermediate 31 having the following structure:

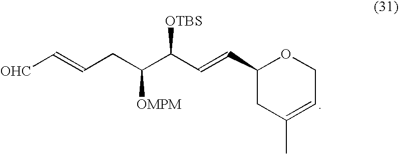

For example, a synthetic approach combining bromide intermediate 1 and enal 31 may lead to the formation of macrolide 33, as depicted in Scheme 7. Analog 33 may be further functionalized. For example, as depicted in Scheme 7, the triple bond may be partially hydrogenated to give the corresponding enone 34, which may be subjected to selective epoxidation to give epoxide 35.

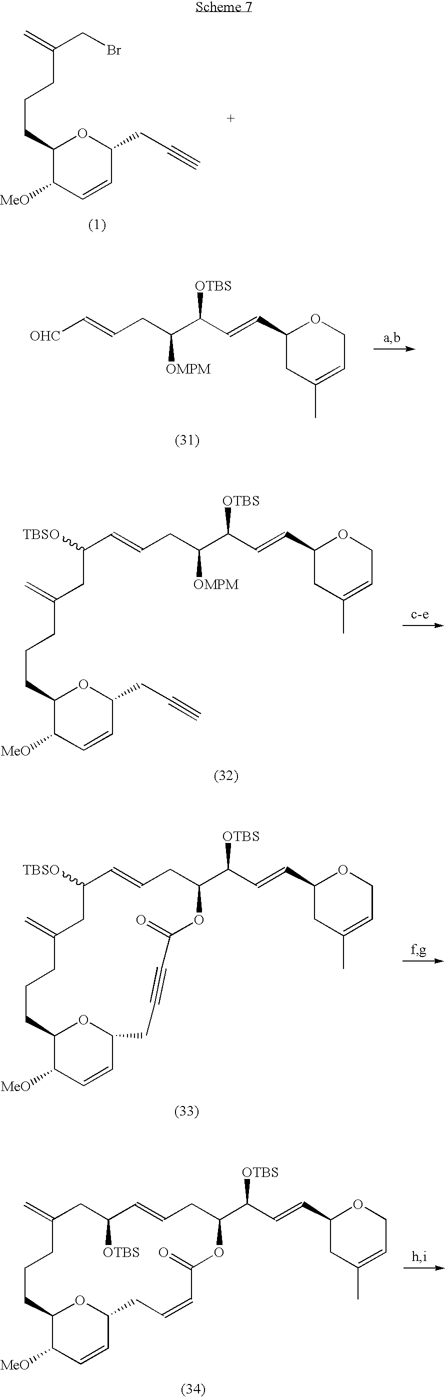

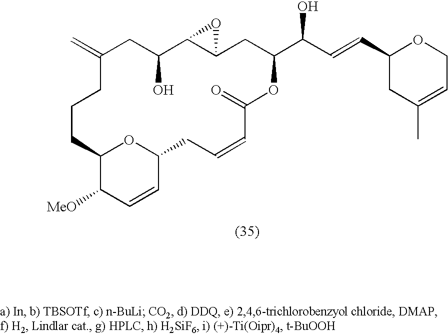

In certain other embodiments, a synthetic approach combining bromide intermediate 7 and enal 31 may lead to the formation of macrolide 39a, as depicted in Scheme 8. Analog 39a may be subjected to selective epoxidation to give epoxide 40.

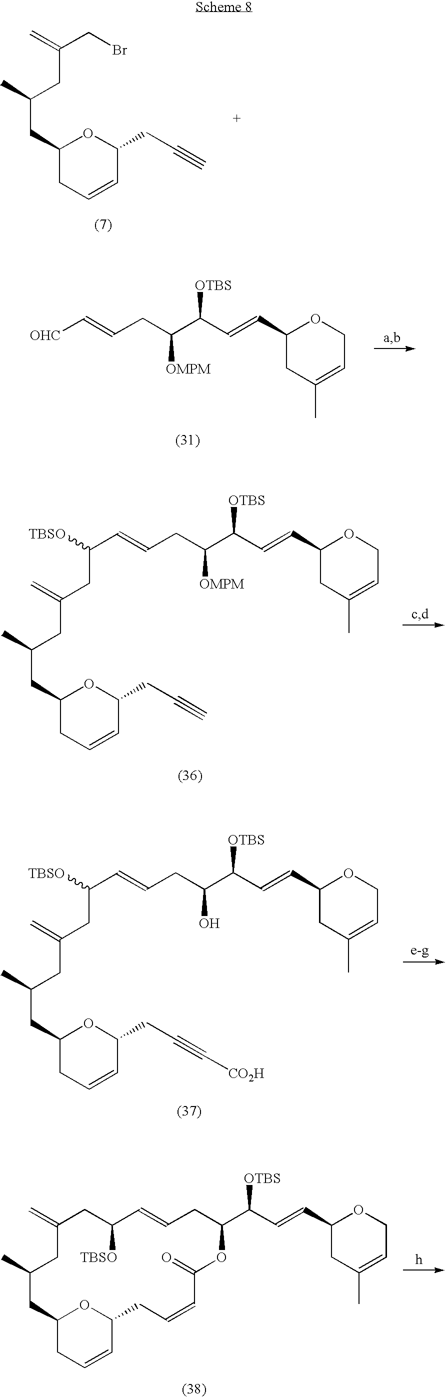

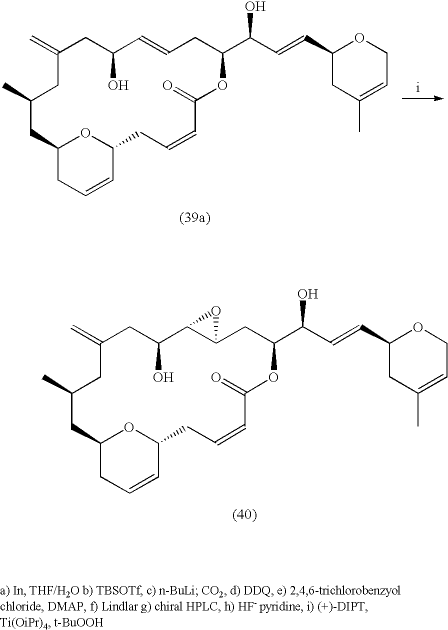

In certain other embodiments, a Homer-Wadsworth-Emmons approach may be used, as depicted in Scheme 9. Such an approach would give rise to a mixture of diastereomers 39a-d, which may be separated by a suitable separation technique (e.g., HPLC).

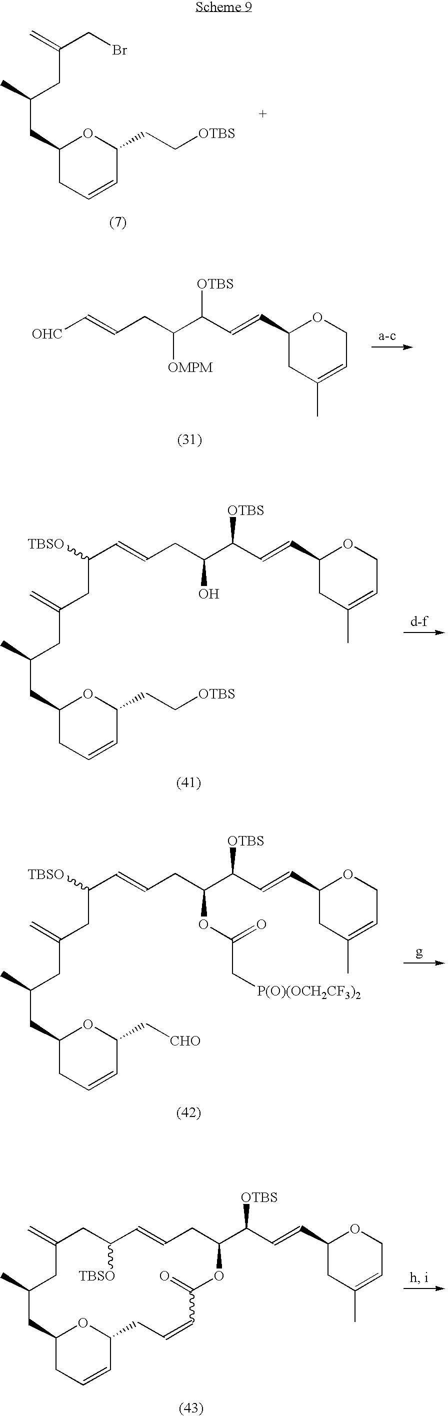

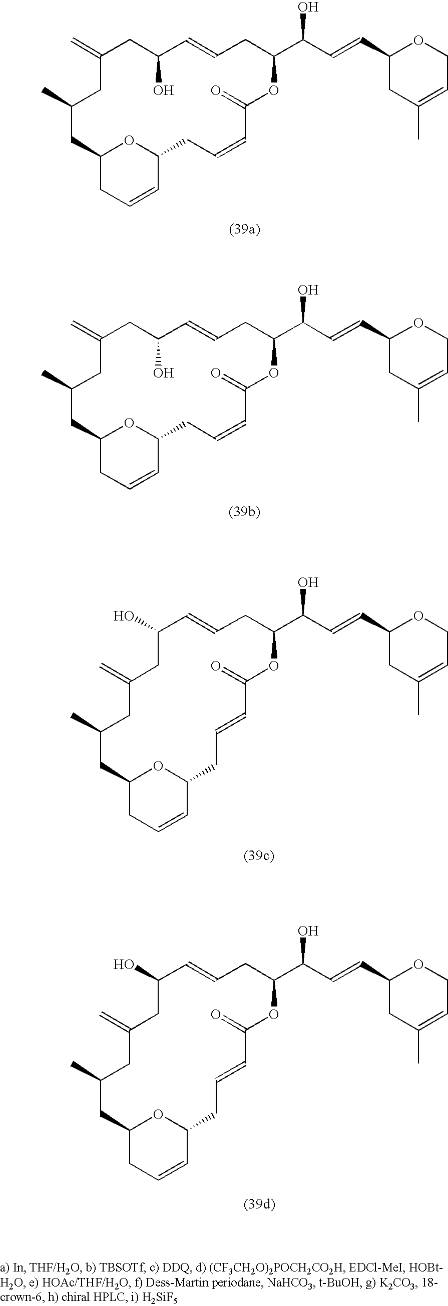

In certain exemplary embodiments, (CF₃CH₂O)₂POCH₂CO₂H (47) may be obtained through the methodology depicted in Scheme 10.

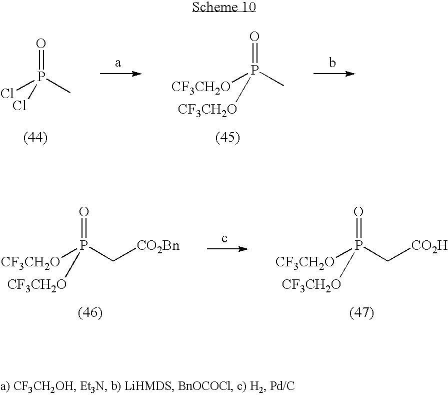

In certain exemplary embodiments, intermediate 31 may be obtained through the methodology depicted in Scheme 11.

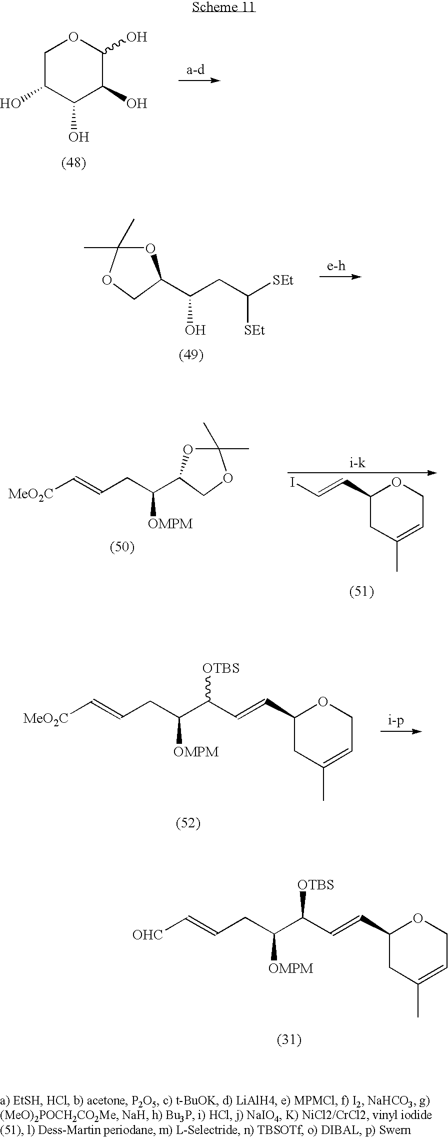

In certain exemplary embodiments, vinyl iodide 51 may be obtained through the methodology depicted in Scheme 12.

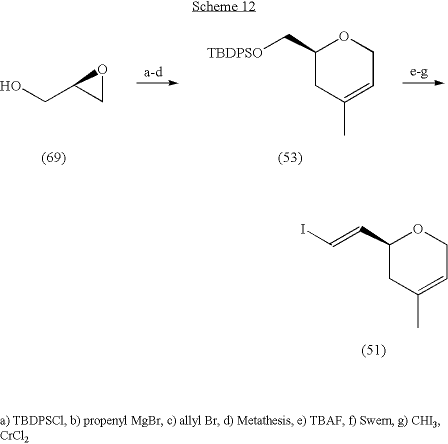

In certain other exemplary embodiments, intermediate 31 may be obtained via a Homer-Wadsworth-Emmons reaction, as depicted in Scheme 13.

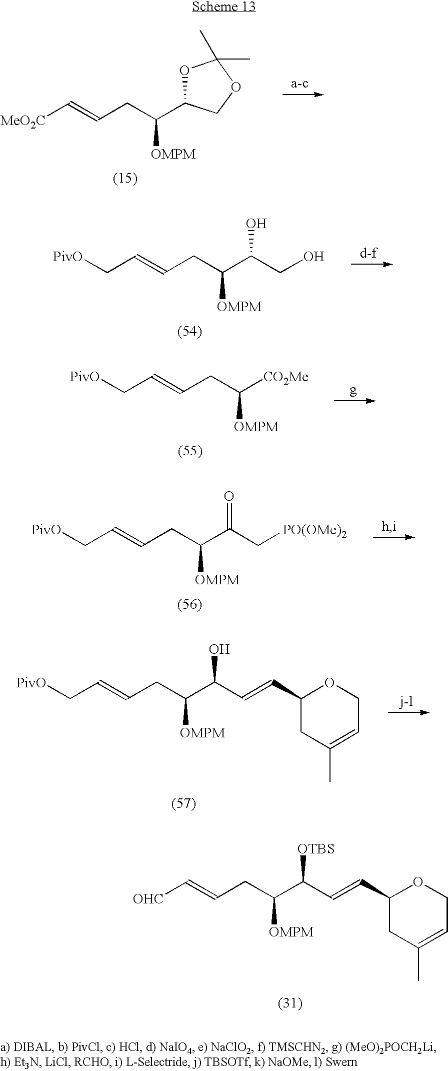

In yet other embodiments, compounds of formula I wherein —K-L-M-R₁₀ is a moiety having, the structure:

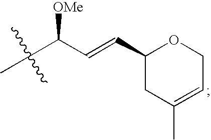

may be obtained by using intermediate 60 having the following structure:

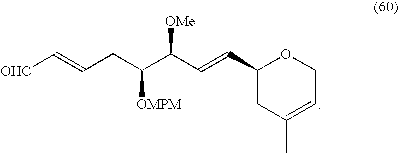

For example, a synthetic approach combining bromide intermediate 7 and enal 60 may lead to the formation of macrolide 62, as depicted in Scheme 14.

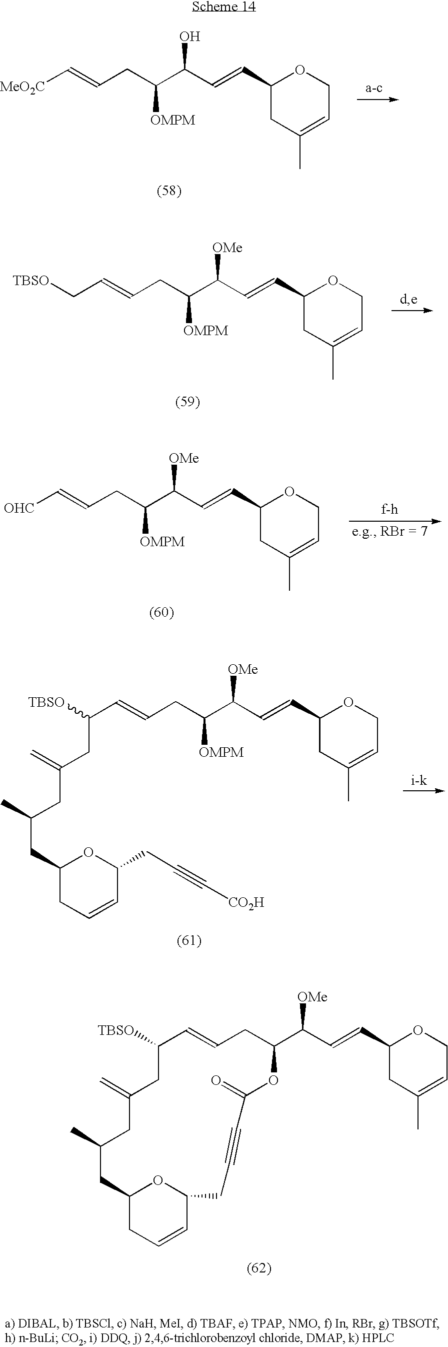

Analog 62 may be further functionalized. For example, as depicted in Scheme 15, analog 62 may be epoxidized to give epoxide 63. In addition, the triple bond in analog 63 may be epoxidized and partially or fully hydrogenated to give the corresponding analogs 64 and 65, respectively.

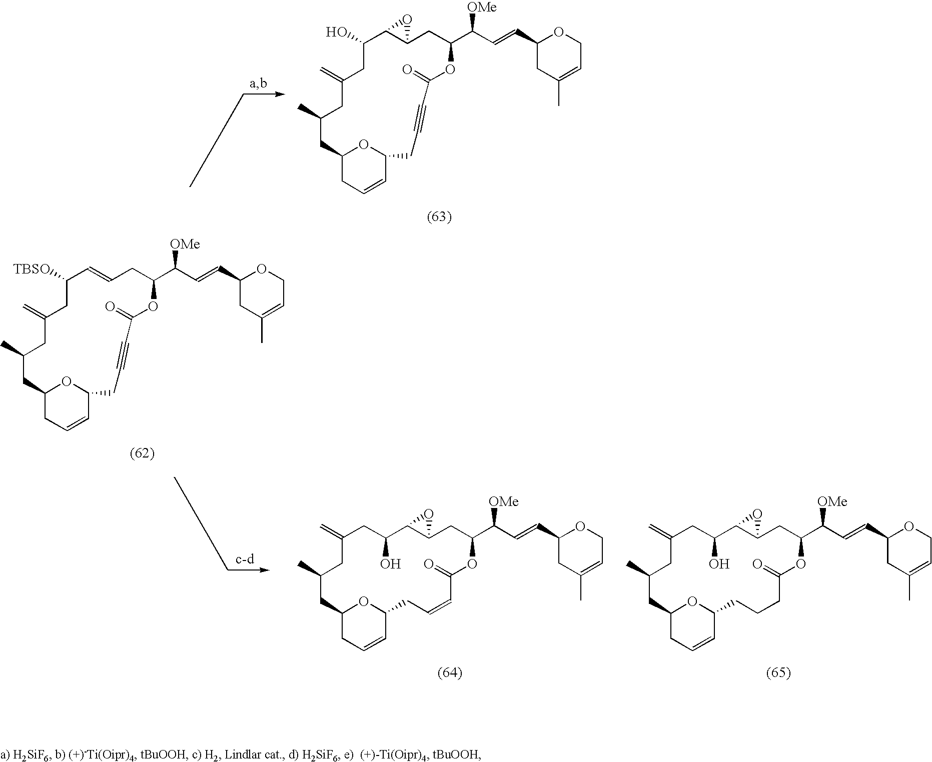

Exemplary synthetic methods for the preparation of compounds of formula (I) where Z is NH are described in the Exemplification section. Other synthetic approaches will be apparent to the skilled practitioner.

Compounds of formula (I) where Z is S may be prepared, for example, by treating the appropriate C19 alcohol compound with P2S5 or Lawesson's reagent. Other synthetic approaches will be apparent to the skilled practitioner.

Compounds of formula (I) where Z is CH may be prepared, for example, according to the methodolody depicted in Scheme 15. Other synthetic approaches will be apparent to the skilled practitioner.

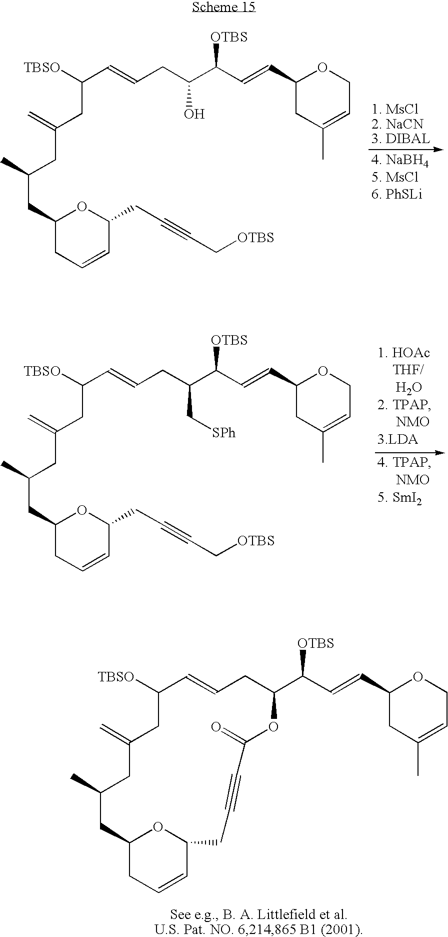

Compounds of formula (I) where R₉ₐ and R_{9b}, taken together with X₁, form an aromatic or heteroaromatic moiety may be prepared, for example, according to the methodolody depicted in Scheme 16. Other synthetic approaches will be apparent to the skilled practitioner.

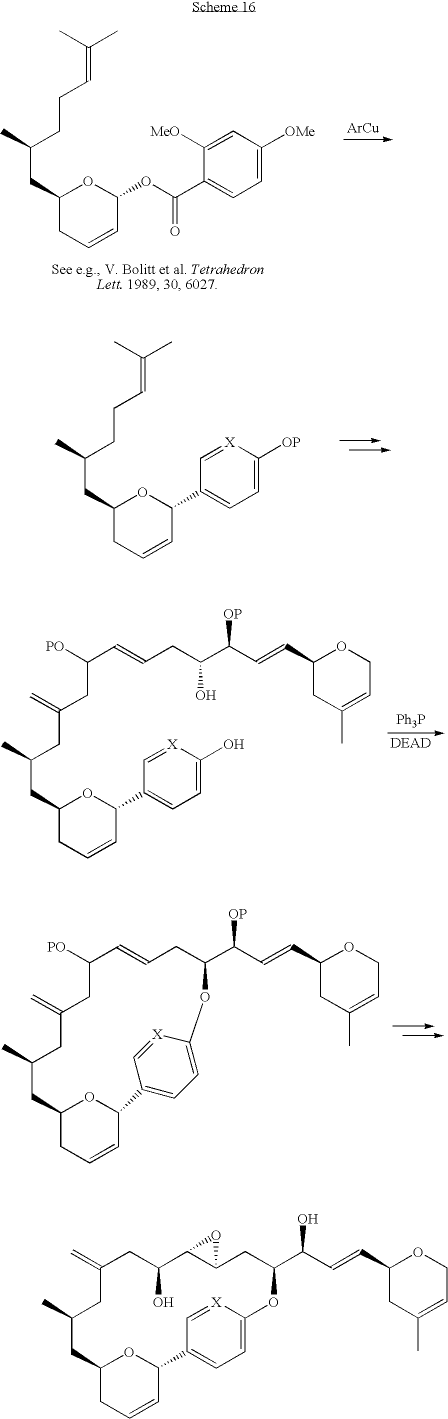

Compounds of formula (I) where R₉ₐ and R_{9b}, taken together with X₁, form a phenyl moiety may be prepared, for example, according to the methodolody depicted in Scheme 17. Other synthetic approaches will be apparent to the skilled practitioner.

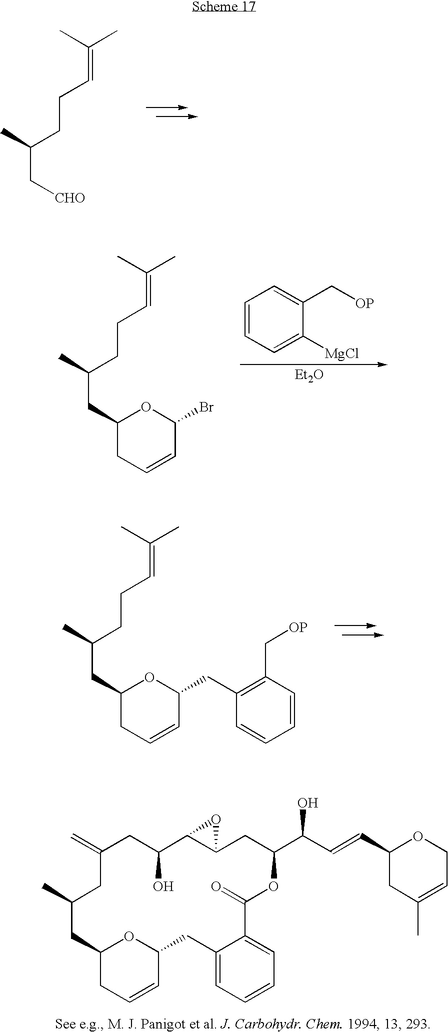

Compounds of formula (I) where R₉ₐ and R_{9b}, taken together with X₁, form a phenyl moiety may be prepared, for example, according to the methodolody depicted in Scheme 18. Other synthetic approaches will be apparent to the skilled practitioner.

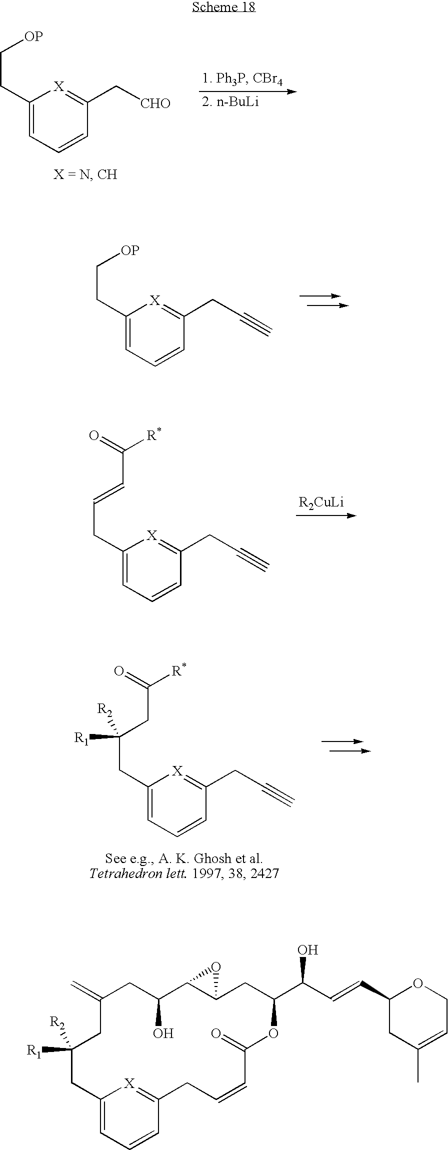

Compounds of formula (I) where —K-L-M together represent a diol side chain may be prepared, for example, according to the methodolody depicted in Scheme 19. Other synthetic approaches will be apparent to the skilled practitioner.

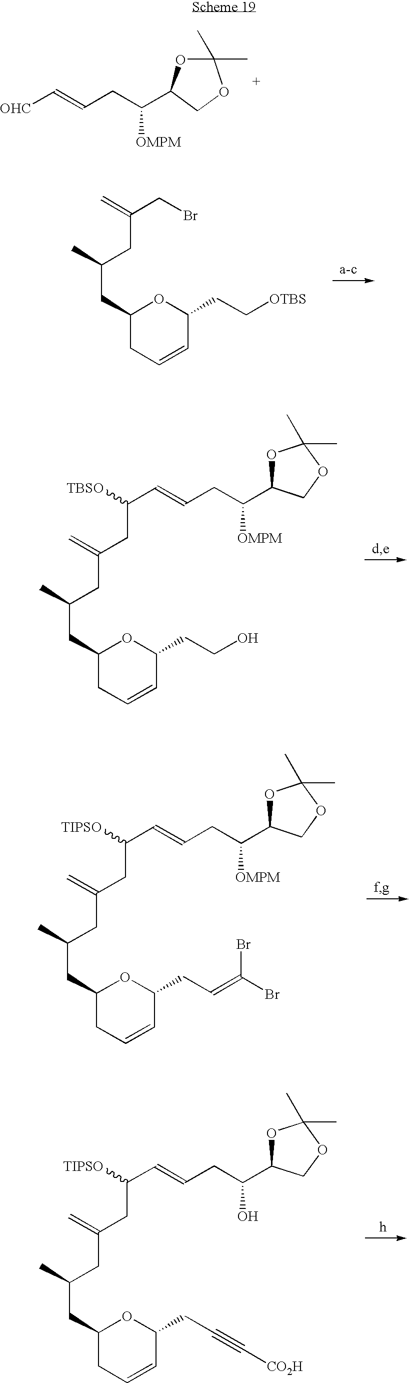

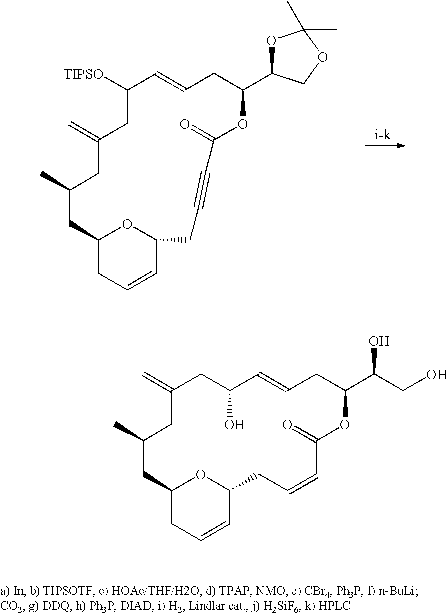

Diversification:

It will also be appreciated that each of the components used in the synthesis of inventive compounds can be diversified either before synthesis or alternatively after the construction of the core structure of formula (I). As used herein, the term “diversifying” or “diversify” means reacting an inventive compound (I) or any of the precursor fragments (or any classes or subclasses thereof) at one or more reactive sites to modify a functional moiety or to add a functional moiety (e.g., nucleophilic addition of a substrate). Described generally herein are a variety of schemes to assist the reader in the synthesis of a variety of compounds, either by diversification of the intermediate components or by diversification of the core structures as described herein, and classes and subclasses thereof. It will be appreciated that a variety of diversification reactions can be employed to generate compounds other than those described in the Exemplification herein. As but a few examples, where a double bond is present in the compound structure, epoxidation and aziridation can be conducted to generate epoxide and aziridine derivatives of compounds described herein. For additional guidance available in the art, the practitioner is directed to “Advanced Organic Chemistry”, March, J. John Wiley & Sons, 2001, 5^{th} ed., the entire contents of which are hereby incorporated by reference.

The skilled practitioner will know how to select reagents, staring materials and reaction conditions to make a variety of analogues and derivatives. The exemplary synthetic methodology described above is a highly efficient approach, and allows access to a variety of Laulimalide analogues and derivatives in quantities sufficient for in vivo testing.

3) Pharmaceutical Compositions

As discussed above this invention provides novel compounds that have biological properties useful for the treatment of disorders associated with cellular hyperproliferation.

Accordingly, in another aspect of the present invention, pharmaceutical compositions are provided, which comprise any one of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved chemotherapeutic agent, anti-inflammatory agent, or it may be any one of a number of agents undergoing approval in the Food and Drug Administration that ultimately obtain approval for the treatment of any disorder associated with cellular hyperproliferation. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof

Optionally, the composition may include one or more additional microtubule-stabilizing agents. Representative examples of other microtubule-stabilizing agents include but are not limited to: taxanes (e.g., paclitaxel and docetaxel), epothilone, camptothecin, eleutherobin, sarcodictyins, discodermolide, and derivatives thereof. Formulations for taxanes are described by, for example, PCT publication no. WO 99/62510, which is incorporated herein by reference in its entirety.

When the composition is used to treat psoriasis and dermatitis, the composition optionally may contain therapeutically effective amount of one or more compounds that are used to treat psoriasis and dermatitis including but not limited to: cyclosporine; methotrexate; tamoxifen; forskolin and analogs; tar derivatives; steroids; vitamin A and its derivatives; vitamin D and its derivatives including 1-alpha-hydroxyl-cholecalciferol, 1,25-dihydrlxyl-cholecalciferol, 24,25-dihydroxy-cholecalciferol, 1,24-dihydroxy-cholecalciferol and calcipotriol (MC 903); and beta agonists such as terbutaline.

A wide variety of carriers may be selected of either polymeric or non-polymeric origin which may be biodegradable or non-biodegradable. Examples of suitable carriers are described in published U.S. Patent Application 2002/0128471, paragraphs [0111] through [0123] which are incorporated herein by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66:1-19 (1977), incorporated herein by reference. The salts can be prepared in situ during, the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting, agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredients) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/druglistframe)—See Appendix A. Examples of chemotherapeutic agents which can be used in combination with the inventive compounds are also described in PCT Publication WO 03/076445, which is incorporated herein by reference in its entirety.

In certain embodiments, the pharmaceutical compositions of the present invention further comprise one or more additional therapeutically active ingredients (e.g., chemotherapeutic and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

In certain embodiments, compounds of the invention are useful for the treatment of psoriasis and pharmaceutical compositions containing them may be administered in combination with any of the antipsoriatic therapies or therapeutic agents known in the art. For example, therapies or antipsoriatic agents that may be used in combination with the inventive compounds of the present invention include Ultraviolet light treatment (e.g., sunlight), lubricants, keratolytics, emollients (e.g., Aqueous Cream, E45, and Emulsifying ointment), ammoniated mercury, topical vitamin D analogs (e.g., Calcipotriol (Dovonex), Tacalcitol (Curatoderm)), dithranol (e.g., Dithrocream and Miconal), tar (e.g., Alphosyl, anthralin), topical steroids (e.g., corticosteroids, halobetasol), topical retinoids zorac, Tazarotene), systemic antimetabolites (e.g., oral methotrexate), immunosuppressive drugs (e.g., oral cyclosporine, tacrolimus, mycophenolate, and mofetil) and oral retinoids (e.g., acitretin).

4) Research Uses, Pharmaceutical Uses and Methods of Treatment

Research Uses

According to the present invention, the inventive compounds may be assayed in any of the available assays known in the art for identifying compounds having antiangiogenic activity and/or antiproliferative activity. For example, the assay may be cellular or non-cellular, in vivo or in vitro, high- or low-throughput format, etc.

Thus, in one aspect, compounds of this invention which are of particular interest include those which:
exhibit activity as microtubule-stabilizing agents;
exhibit an antiproliferative effect on solid tumors; and/or
exhibit a favorable therapeutic profile (e.g., safety, efficacy, and stability).

As detailed in the exemplification herein, in assays to determine the ability of compounds to stabilize microtubule certain inventive compounds exhibit IC₅₀ values≦50 μM. In certain other embodiments, inventive compounds exhibit IC₅₀ values≦40 μM. In certain other embodiments, inventive compounds exhibit IC₅₀ values≦30 μM. In certain other embodiments, inventive compounds exhibit IC₅₀ values≦20 μM. In certain other embodiments, inventive compounds exhibit IC₅₀ values≦10 μM. In certain other embodiments, inventive compounds exhibit IC₅₀ values≦7.5 μM. In certain embodiments, inventive compounds exhibit IC₅₀ values≦5 μM. In certain other embodiments, inventive compounds exhibit IC₅₀ values≦2.5 μM. In certain embodiments, inventive compounds exhibit IC₅₀ values≦1 μM. In certain embodiments, inventive compounds exhibit IC₅₀ values≦0.75 μM. In certain embodiments, inventive compounds exhibit IC₅₀ values≦0.5 μM. In certain embodiments, inventive compounds exhibit IC₅₀ values≦0.25 μM. In certain embodiments, inventive compounds exhibit IC₅₀ values≦0.1 μM. In other embodiments, exemplary compounds exhibit IC₅₀ values≦75 nM. In other embodiments, exemplary compounds exhibit IC₅₀ values≦50 nM. In other embodiments, exemplary compounds exhibit IC₅₀ values≦25 nM. In other embodiments, exemplary compounds exhibit IC₅₀ values≦10 nM. In other embodiments, exemplary compounds exhibit IC₅₀ values≦5 nM.

As detailed in the exemplification herein, in assays to determine the ability of compounds to inhibit tumor cell proliferation, certain inventive compounds exhibit IC₅₀ values≦200 μM. In certain other embodiments, inventive compounds exhibit IC₅₀ values≦150 μM. In certain other embodiments, inventive compounds exhibit IC₅₀ values≦100 μM. In certain other embodiments, inventive compounds exhibit IC₅₀ values≦50 μM. In certain other embodiments, inventive compounds exhibit IC₅₀ values≦10 μM. In certain other embodiments, inventive compounds exhibit IC₅₀ values≦7.5 μM. In certain embodiments, inventive compounds exhibit IC₅₀ values≦5 μM. In certain other embodiments, inventive compounds exhibit IC₅₀ values≦2.5 μM. In certain embodiments, inventive compounds exhibit IC₅₀ values≦1 μM. In certain embodiments, inventive compounds exhibit IC₅₀ values≦0.75 μM. In certain embodiments, inventive compounds exhibit IC₅₀ values≦0.5 μM. In certain embodiments, inventive compounds exhibit IC₅₀ values≦0.25 μM. In certain embodiments, inventive compounds exhibit IC₅₀ values≦0.1 μM. In other embodiments, exemplary compounds exhibit IC₅₀ values≦75 nM. In other embodiments, exemplary compounds exhibit IC₅₀ values≦50 nM. In other embodiments, exemplary compounds exhibit IC₅₀ values≦25 nM. In other embodiments, exemplary compounds exhibit IC₅₀ values≦10 nM. In other embodiments, exemplary compounds exhibit IC₅₀ values≦5 nM.

Pharmaceutical Uses and Methods of Treatment

In general, methods of using the compounds of the present invention comprise administering to a subject in need thereof a therapeutically effective amount of a compound of the present invention. Diseases that may be treated with the compounds of the present invention are those that are characterized by cellular hyperproliferation, such as cancers, tumors, and inflammatory disorders. Illustrative examples of inflammatory disorders include, for example, atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis, irritant dermatitis, allergic dermatitis), periodontal disease (i.e., periodontitis), polycystic kidney disease, and type I diabetes.

Accordingly, in another aspect of the invention, methods for the treatment of cancer are provided comprising administering a therapeutically effective amount of a compound of formula (I), as described herein, to a subject in need thereof. In certain embodiments, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result.

In certain embodiments, the method involves the administration of a therapeutically effective amount of the compound or a pharmaceutically acceptable derivative thereof to a subject (including, but not limited to a human or animal) in need of it. In certain embodiments, the inventive compounds as useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric-cancer, bladder cancer, uterine cancer, kidney cancer, testicular cancer, stomach cancer, brain cancer, liver cancer, or esophageal cancer).

In certain embodiments, the inventive anticancer agents are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer, to name a few. In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents are active against solid tumors.

Utilizing the inventive compounds, compositions and methods provided herein, a wide variety of inflammatory skin diseases can be treated or prevented. For example, within one embodiment of the invention an inflammatory skin disease such as psoriasis or eczema may be treated or prevented by delivering, to a site of inflammation (or a potential site of inflammation) an agent that inhibits microtubule function. Briefly, skin cells are genetically programmed to follow two possible programs—normal growth or wound healing. In the normal growth pattern, skin cells are created in the basal cell layer and then move up through the epidermis to the skin surface. Dead cells are shed from healthy skin at the same rate new cells are created. The turnover time (i.e., time from cell birth to death) for normal skin cells is approximately 28 days. During wound healing, accelerated growth and repair is triggered resulting in rapid turnover of skin cells (to replace and repair the wound), increased blood supply (to meet the increased metabolic needs associated with growth) and localized inflammation.

In many respects, psoriasis is similar to an exaggerated wound healing process where skin cells (called “keratinocytes”) are created and pushed to the skin surface in as little as 2-4 days. Psoriasis occurs when skin cells hyperproliferate and the surface skin cannot shed the dead cells fast enough. The excess keratinocytes build up and form elevated, scaly lesions. This growth is supported by new blood vessels in the dermis (the support tissue beneath the epidermis) that are established to provide the nutrients necessary to support the hyperproliferating keratinocytes. At the same time, lymphocytes, neutrophils and macrophage invade the tissue, creating inflammation, swelling and soreness, and potentially producing growth factors that augment the rapid proliferation of the keratinocytes. All these cells (keratinocytes, vascular endothelial cells and white blood cells) produce tissue degrading enzymes or proteinases that aid in the progression of the disease and the destruction of surrounding tissue.

Utilizing the compositions of the present invention, inflammatory skin lesions may be treated. In particular, compounds of the invention are administered directly to the site of inflammation (or a potential site of inflammation), in order to treat or prevent the disease. The one or more inventive compounds may be delivered as a composition along with a polymeric carrier, or in a liposome, cream or ointment formulation as discussed previously. Within preferred embodiments of the invention, the compounds or compositions are delivered either topically, or by subcutaneous administration. An effective therapy for psoriasis will achieve at least one of the following: decrease the number and severity of skin lesions, decrease the frequency or duration of active disease exacerbations, increase the amount of time spent in remission (i.e., periods when the patient is symptom-free) and/or decrease the severity or duration of associated symptoms (e.g., joint pain and swelling, axial skeletal pain, bowel symptoms). Clinically the treatment will result in a reduction in the size or number of skin lesions, diminution of cutaneous symptoms (pain, burning and bleeding of the affected skin) and/or a reduction in associated symptoms (e.g., joint redness, heat, swelling, diarrhea, abdominal pain). Pathologically a microtubule-stabilizing agent will produce at least one of the following: inhibition of keratinocyte proliferation, reduction of skin inflammation (for example, by impacting on: attraction and growth factors, antigen presentation, production of reactive oxygen species and matrix metalloproteinases), and inhibition of dermal angiogenesis.

The compounds of the invention can be administered in any manner sufficient to achieve the above end points. In certain embodiments, the methods include topical and systemic administration. Patients with localized disease can be administered a topical cream, ointment or emollient applied directly to the psoriatic lesions. For example, a topical cream containing 0.001% to 10% of an inventive compound by weight is administered depending upon severity of the disease and the patient's response to treatment. In certain embodiments, a topical preparation containing an inventive compound at 0.01% to 1% by weight is administered to psoriatic lesions. Alternatively, direct intracutaneous injection of an inventive compound in a suitable pharmaceutical vehicle can be used for the management of individual lesions. In patients with widespread disease or extracutaneous symptoms (e.g., psoriatic arthritis, Reiter's syndrome, associated spondylitis, associated inflammatory bowel disease) systemic treatment can be administered. For example, intermittent treatments with an intravenous formulation can be administered at a dose of 10 to 75 mg/m² of a compound of the present invention depending, upon therapeutic response and patient tolerance. An equivalent oral preparation would also be suitable for this indication.

Other dermatological conditions that can also benefit from topical administration of inventive compounds include: eczematous disease (atopic dermatitis, contact dermatitis, eczema), immunobullous disease, pre-malignant epithelial tumors, basal cell carcinoma, squamous cell carcinoma, keratocanthoma, malignant melanoma and viral warts. Topical creams, ointments, and emollients containing 0.001% to 10% inventive compound by weight can be suitable for the management of these conditions.

Compounds of the invention may be utilized to treat or prevent chronic inflammatory neurological disorders, such as multiple sclerosis. Briefly, multiple sclerosis (“MS”) is a devastating demyelinating disease of the human central nervous system. Although its etiology and pathogenesis is not known, genetic, immunological and environmental factors are believed to play a role. In the course of the disease, there is a progressive demyelination in the brain of MS patients resulting, in the loss of motor function. Although the exact mechanisms involved in the loss of myelin are not understood, there is an increase in astrocyte proliferation and accumulation in the areas of myelin destruction. At these sites, there is macrophage-like activity and increased protease activity which is at least partially responsible for degradation of the myelin sheath.

Compounds of the present invention can be administered to the site of inflammation (or a potential site of inflammation), in order to treat or prevent the disease. Such agents may, within certain embodiments, be delivered as a composition along with a polymeric carrier, or in a liposome formulation as previously. Within certain embodiments of the invention, the agents or compositions may be administered orally, intravenously, or by direct administration (preferably with ultrasound, CT, fluoroscopic, MRI or endoscopic guidance) to the disease site. An effective therapy for multiple sclerosis will accomplish one or more of the following: decrease the severity of symptoms; decrease the duration of disease exacerbations; increase the frequency and duration of disease remission/symptom-free periods; prevent fixed impairment and disability; and/or prevent/attenuate chronic progression of the disease. Clinically, this would result in improvement in visual symptoms (visual loss, diplopia), gait disorders (weakness, axial instability, sensory loss, spasticity, hyperreflexia, loss of dexterity), upper extremity dysfunction (weakness, spasticity, sensory loss), bladder dysfunction (urgency, incontinence, hesitancy, incomplete emptying), depression, emotional lability, and cognitive impairment. Pathologically the treatment reduces one or more of the following, such as myelin loss, breakdown of the blood-brain barrier, perivascular infiltration of mononuclear cells, immunologic abnormalities, gliotic scar formation and astrocyte proliferation, metalloproteinase production, and impaired conduction velocity.

The microtubule-stabilizing agent can be administered in any manner sufficient to achieve the above endpoints. However, preferred methods of administration include intravenous, oral, or subcutaneous, intramuscular or intrathecal injection. The microtubule-stabilizing agent can be administered as a chronic low dose therapy to prevent disease progression, prolong, disease remission or decrease symptoms in active disease. Alternatively, the therapeutic agent can be administered in higher doses as a “pulse” therapy to induce remission in acutely active disease. The minimum dose capable of achieving these endpoints can be used and can vary according to patient, severity of disease, formulation of the administered agent, and route of administration. For example, preferred embodiments would include 10 to 75 mg/m² of an inventive compound once every 1 to 4 weeks. 10 to 75 mg/m² daily, as tolerated, or 10 to 175 mg/m² once weekly, as tolerated or until symptoms subside.

Inflammatory arthritis is a serious health problem in developed countries, particularly given the increasing number of aged individuals. For example, one form of inflammatory arthritis, rheumatoid arthritis (“RA”) is a multisystem chronic, relapsing, inflammatory disease of unknown cause. Although many organs can be affected, RA is basically a severe form of chronic synovitis that sometimes leads to destruction and ankyiosis of affected joints (Robbins Pathological Basis of Disease, by R. S. Cotran, V. Kumar, and S. L. Robbins, W. B. Saunders Co., 1989). Pathologically, the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and, eventually the pannus grows to fill the joint space. The pannus develops an extensive network of new blood vessels through the process of angiogenesis that is essential to the evolution of the synovitis. The release of digestive enzymes (matrix metalloproteinases such as collagenase, stromelysin, and the like) and other mediators of the inflammatory process (e.g., hydrogen peroxide, superoxides, lysosomal enzymes, and products of arachadonic acid metabolism) from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis and ultimately bony ankylosis, of the involved joint. It is generally believed, but not conclusively proven, that RA is an autoimmune disease, and that many different arthrogenic stimuli activate the immune response in the immunogenetically susceptible host. Both exogenous infectious agents (Ebstein-Barr virus, rubella virus, cytomegalovirus, herpes virus, human T-cell lymphotropic virus, Mycoplasma, and others) and endogenous proteins (collagen, proteoglycans, altered immunoglobulins) have been implicated as the causative agent that triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive inappropriate immune response directed against the host tissues (e.g., antibodies directed against type II collagen, antibodies directed against the Fc portion of autologous IgG (called “Rheumatoid Factor”)). This further amplifies the immune response and hastens the destruction of the cartilage tissue. Once this cascade is initiated numerous mediators of cartilage destruction are responsible for the progression of rheumatoid arthritis.

Thus, within one aspect of the present invention, methods are provided for treating or preventing inflammatory arthritis (e.g., rheumatoid arthritis) comprising the step of administering to a patient a therapeutically effective amount of a microtubule-stabilizing agent. Inflammatory arthritis includes a variety of conditions including, but not limited to, rheumatoid arthritis, systemic lupus erythematosus, systemic sclerosis (sclerodemia), mixed connective tissue disease, Sjogren's syndrome, ankylosing spondylitis, Behcet's syndrome, sarcoidosis, and osteoarthritis—all of which feature inflamed, painful joints as a prominent symptom.

Within a preferred embodiment of the invention, microtubule-stabilizing agents may be administered directly to a joint by intra-articular injection, as a surgical paste or administered by another route, e.g., systemically or orally. Such agents may, within certain embodiments, be delivered as a composition along, with a polymeric carrier, or in a liposome formulation as discussed previously.

An effective microtubule-stabilizing therapy for inflammatory arthritis will accomplish one or more of the following: (i) decrease the severity of symptoms (pain, swelling and tenderness of affected joints; morning stiffness. weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease. Pathologically, an effective microtubule-stabilizing therapy for inflammatory arthritis will produce at least one of the following: (i) decrease the inflammatory response, (ii) disrupt the activity of inflammatory cytokines (such as IL-I, TNFa, FGF, VEGF), (iii) inhibit synoviocyte proliferation, (iv) block matrix metalloproteinase activity, and/or (v) inhibit angiogenesis. A microtubule-stabilizing agent will be administered systemically (orally, intravenously, or by intramuscular or subcutaneous injection) in the minimum dose to achieve the above mentioned results. For patients with only a small number of joints affected, or with disease more prominent in a limited number of joints, the microtubule-stabilizing agent can be directly injected (intra-articular injection) into the affected joints. The microtubule-stabilizing agent can be administered in any manner sufficient to achieve the above endpoints. However, preferred methods of administration include intravenous, oral, or subcutaneous, intramuscular or intra-articular injection. The microtubule-stabilizing agent can be administered as a chronic low dose therapy to prevent disease progression, prolong disease remission, or decrease symptoms in active disease.

Alternatively, the therapeutic agent can be administered in higher doses as a “pulse” therapy to induce remission in acutely active disease. The minimum dose capable of achieving these endpoints can be used and can vary according to patient, severity of disease, formulation of the administered agent, and route of administration. For example, preferred embodiments would include 10 to 75 mg/m² of an inventive compound once every 1 to 4 weeks, 10 to 75 mg/m² daily, as tolerated, or 10 to 175 mg/m² once weekly, as tolerated or until symptoms subside.

As discussed above, the inventive compounds also find use in the prevention of restenosis of blood vessels subject to traumas such as angioplasty and stenting. For example, it is contemplated that the compounds of the invention will be useful as a coating for implanted medical devices, such as tubings, shunts, catheters, artificial implants, pins, electrical implants such as pacemakers, and especially for arterial or venous stents, including balloon-expandable stents. In certain embodiments inventive compounds may be bound to an implantable medical device, or alternatively, may be passively adsorbed to the surface of the implantable device. In certain other embodiments, the inventive compounds may be formulated to be contained within, or, adapted to release by a surgical or medical device or implant, such as, for example, stents, sutures, indwelling catheters, prosthesis, and the like.

In certain exemplary embodiments, the inventive compounds may be used as coating for stents. A stent is typically an open tubular structure that has a pattern (or patterns) of apertures extending from the outer surface of the stent to the lumen. It is commonplace to make stents of biocompatible metallic materials, with the patterns cut on the surface with a laser machine. The stent can be electro-polished to minimize surface irregularities since these irregularities can trigger an adverse biological response. However, stents may still stimulate foreign body reactions that result in thrombosis or restenosis. To avoid these complications, a variety of stent coatings and compositions have been proposed in the prior art literature both to reduce the incidence of these complications or other complications and restore tissue function by itself or by delivering therapeutic compound to the lumen. For example, drugs having antiproliferative and anti-inflammatory activities have been evaluated as stent coatings, and have shown promise in preventing retenosis (See, for example, Presbitero P. et al., “Drug eluting stents do they make the difference?”, Minerva Cardioangiol, 2002, 50(5):431-442; Ruygrok P. N. et al., “Rapamycin in cardiovascular medicine”, Intern. Med. J., 2003, 33(3):103-109; and Marx S. O. et al., “Bench to bedside: the development of rapamycin and its application to stent restenosis”, Circulation, 2001, 104(8):852-855, each of these references is incorporated herein by reference in its entirety). Accordingly, without wishing to be bound to any particular theory, Applicant proposes that inventive compounds having anti-inflammatory and/or antiproliferative effects can be used as stent coatings and/or in stent drug delivery devices, inter alia for the prevention of restenosis or reduction of restenosis rate. A variety of compositions and methods related to stent coating and/or local stent drug delivery for preventing restenosis are known in the art (see, for example, U.S. Pat. Nos. 6,517,889; 6,273,913; 6,258,121; 6,251,136; 6,248,127; 6,231,600; 6,203,551; 6,153,252; 6,071,305; 5,891,507; 5,837,313 and published U.S. patent application No.: US2001/0027340, each of which is incorporated herein by reference in its entirety). For example, stents may be coated with polymer-drug conjugates by dipping the stent in polymer-drug solution or spraying the stent with such a solution. In certain embodiment, suitable materials for the implantable device include biocompatible and nontoxic materials, and may be chosen from the metals such as nickel-titanium alloys, steel, or biocompatible polymers, hydrogels, polyurethanes, polyethylenes, ethylenevinyl acetate copolymers, etc. In certain embodiments, the inventive compound, is coated onto a stent for insertion into an artery or vein following balloon angioplasty.

The invention may be described therefore, in certain broad aspects as a method of inhibiting arterial restenosis or arterial occlusion following vascular trauma comprising administering to a subject in need thereof, a composition comprising an inventive compound conjugated to a suitable polymer or polymeric material. In the practice of the method, the subject may be a coronary bypass, vascular surgery, organ transplant or coronary or any other arterial angioplasty patient, for example, and the composition may be administered directly, intravenously, or even coated on a stent to be implanted at the sight of vascular trauma.

In another aspect, the invention encompasses implants and surgical or medical devices, including stents and grafts, coated with or otherwise constructed to contain and/or release any of the inventive compounds disclosed herein. In certain embodiments, the compounds have anti-inflammatory and/or antiproliferative activities. In certain other embodiments, the compounds inhibit smooth muscle cell proliferation. Representative examples of the inventive implants and surgical or medical devices include cardiovascular devices (e.g., implantable venous catheters, venous ports, tunneled venous catheters, chronic infusion lines or ports, including hepatic artery infusion catheters, pacemaker wires, implantable defibrillators); neurologic/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, implantable meshes for hernias, suspensions or solid implants to prevent surgical adhesions, including meshes); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy); phthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants); otolaryngology devices (e.g., ossicular implants, Eustachian tube splints or stents for glue ear or chronic otitis as an alternative to transtempanic drains); plastic surgery implants (e.g., prevention of fibrous contracture in response to gel- or saline-containing breast implants in the subpectoral or subglandular approaches or post-mastectomy, or chin implants), and orthopedic implants (e.g., cemented orthopedic prostheses).

Implants and other surgical or medical devices may be coated with (or otherwise adapted to release) compositions of the present invention in a variety of manners, including for example: (a) by directly affixing to the implant or device an inventive compound or composition (e.g., by either spraying the implant or device with a polymer/drug film, or by dipping the implant or device into a polymer/drug solution, or by other covalent or noncovalent means); (b) by coating the implant or device with a substance such as a hydrogel which will in turn absorb the inventive compound or composition; (c) by interweaving inventive compound- or composition-coated thread (or the polymer itself formed into a thread) into the implant or device; (d) by inserting the implant or device into a sleeve or mesh which is comprised of or coated with an inventive compound or composition; (e) constructing the implant or device itself with an inventive compound or composition; or (f) by otherwise adapting the implant or device to release the inventive compound. In certain embodiments, the composition should firmly adhere to the implant or device during storage and at the time of insertion. The inventive compound or composition should also preferably not degrade during storage, prior to insertion, or when warmed to body temperature after insertion inside the body (if this is required). In addition, it should preferably coat the implant or device smoothly and evenly, with a uniform distribution of inventive compound, while not changing the stent contour. Within preferred embodiments of the invention, the inventive implant or device should provide a uniform, predictable, prolonged release of the inventive compound or composition into the tissue surrounding the implant or device once it has been deployed. For vascular stents, in addition to the above properties, the composition should not render the stent thrombogenic (causing blood clots to form), or cause significant turbulence in blood flow (more than the stent itself would be expected to cause if it was uncoated).

In the case of stents, a wide variety of stents may be developed to contain and/or release the inventive compounds or compositions provided herein, including esophageal stents, gastrointestinal stents, vascular stents, biliary stents, colonic stents, pancreatic stents, ureteric and urethral stents, lacrimal stents, Eustachian tube stents, fallopian tube stents and tracheal/bronchial stents (See, for example, U.S. Pat. No. 6,515,016, the entire contents of which are incorporated herein by reference). Stents may be readily obtained from commercial sources, or constructed in accordance with well-known techniques. Representative examples of stents include those described in U.S. Pat. No. 4,768,523, entitled “Hydrogel Adhesive”; U.S. Pat. No. 4,776,337, entitled “Expandable Intraluminal Graft, and Method and Apparatus for Implanting and Expandable Intraluminal Graft”; U.S. Pat. No. 5,041,126 entitled “Endovascular Stent and Delivery System”; U.S. Pat. No. 5,052,998 entitled “Indwelling Stent and Method of Use”; U.S. Pat. No. 5,064,435 entitled “Self-Expanding Prosthesis Having Stable Axial Length”; U.S. Pat. No. 5,089,606, entitled “Water-insoluble Polysaccharide Hydrogel Foam for Medical Applications”; U.S. Pat. No. 5,147,370, entitled “Nitinol Stent for Hollow Body Conduits”; U.S. Pat. No. 5,176,626, entitled “Indwelling Stent”; U.S. Pat. No. 5,213,580, entitled “Biodegradable Polymeric Endoluminal Sealing Process”; and U.S. Pat. No. 5,328,471, entitled “Method and Apparatus for Treatment of Focal Disease in Hollow Tubular Organs and Other Tissue Lumens.”

As discussed above, the stent coated with (or otherwise adapted to release) compositions of the present invention may be used to eliminate a vascular obstruction and prevent restenosis or reduce the rate of restenosis. Within other aspects of the present invention, stents coated with (or otherwise adapted to release) compositions of the present invention are provided for expanding the lumen of a body passageway. Specifically, a stent having a generally tubular structure, and a surface coated with (or otherwise adapted to release) an inventive compound or composition may be inserted into the passageway, such that the passageway is expanded. In certain embodiments, the stent coated with (or otherwise adapted to release) compositions of the present invention may be used to eliminate a biliary, gastrointestinal, esophageal, tracheal/bronchial, urethral or vascular obstruction.

In certain other embodiments, methods are provided for using the inventive implants and other surgical or medical devices coated with (or otherwise adapted to release) compounds and compositions of the present invention. In certain embodiments, methods are provided for preventing restenosis, comprising inserting a stent into an obstructed blood vessel, the stent having a generally tubular structure, the surface of the structure being coated with (or otherwise adapted to release) an inventive compound or composition, such that the obstruction is eliminated and the inventive compound or composition is delivered in amounts effective to prevent restenosis. In other embodiments, methods are provided for preventing restenosis, comprising inserting a stent into an obstructed blood vessel, the stent having a generally tubular structure, the surface of the structure being coated with (or otherwise adapted to release) an inventive compound or composition, such that the obstruction is eliminated and the inventive compound or composition is delivered in amounts effective to inhibit smooth muscle cell proliferation.

Within other aspects of the present invention, methods are provided for expanding the lumen of a body passageway, comprising inserting a stent into the passageway, the stent having a generally tubular structure, the surface of the structure being coated with (or otherwise adapted to release) an inventive compound or composition, such that the passageway is expanded. In certain embodiments, the lumen of a body passageway is expanded in order to eliminate a biliary, gastrointestinal, esophageal, tracheal/bronchial, urethral and/or vascular obstruction.

In certain embodiments, methods are provided for eliminating biliary obstructions, comprising inserting a biliary stent into a biliary passageway, the stent having a generally tubular structure, the surface of the structure being coated with (or otherwise adapted to release) an inventive compound or composition, such that the biliary obstruction is eliminated. Briefly, tumor overgrowth of the common bile duct results in progressive cholestatic jaundice which is incompatible with life. Generally, the biliary system which drains bile from the liver into the duodenum is most often obstructed by (1) a tumor composed of bile duct cells (cholangiocarcinoma), (2) a tumor which invades the bile duct (e.g., pancreatic carcinoma), or (3) a tumor which exerts extrinsic pressure and compresses the bile duct (e.g., enlarged lymph nodes). Both primary biliary tumors, as well as other tumors which cause compression of the biliary tree may be treated utilizing stents. Implants and other surgical or medical devices may be coated with (or otherwise adapted to release) compositions of the present invention. One example of primary biliary tumors are adenocarcinomas (which are also called Klatskin tumors when found at the bifurcation of the common hepatic duct). These tumors are also referred to as biliary carcinomas, choledocholangiocarcinomas, or adenocarcinomas of the biliary system. Benign tumors which affect the bile duct (e.g., adenoma of the biliary system), and, in rare cases, squamous cell carcinomas of the bile duct and adenocarcinomas of the gallbladder, may also cause compression of the biliary tree and therefore, result in biliary obstruction. Compression of the biliary tree is most commonly due to tumors of the liver and pancreas which compress and therefore obstruct the ducts. Most of the tumors from the pancreas arise from cells of the pancreatic ducts. This is a highly fatal form of cancer (5% of all cancer deaths; 26,000 new cases per year in the U.S.) with an average of 6 months survival and a 1 year survival rate of only 10%. When these tumors are located in the head of the pancreas they frequently cause biliary obstruction, and this detracts significantly from the quality of life of the patient. While all types of pancreatic tumors are generally referred to as “carcinoma of the pancreas” there are histologic subtypes including: adenocarcinoma, adenosquamous carcinoma, cystadenocarcinoma, and acinar cell carcinoma. Hepatic tumors, as discussed above, may also cause compression of the biliary tree, and therefore cause obstruction of the biliary ducts.

In certain embodiments, a biliary stent is first inserted into a biliary passageway in one of several ways: from the top end by inserting a needle through the abdominal wall and through the liver (a percutaneous transhepatic cholangiogram or “PTC”); from the bottom end by cannulating the bile duct through an endoscope inserted through the mouth, stomach, and duodenum (an endoscopic retrograde cholangiogram or “ERCP”); or by direct incision during a surgical procedure. In certain embodiments, a preinsertion examination, PTC, ERCP, or direct visualization at the time of surgery is performed to determine the appropriate position for stent insertion. A guidewire is then advanced through the lesion, and over this a delivery catheter is passed to allow the stent to be inserted in its collapsed form. If the diagnostic exam was a PTC, the guidewire and delivery catheter is inserted via the abdominal wall, while if the original exam was an ERCP the stent may be placed via the mouth. The stent is then positioned under radiologic, endoscopic, or direct visual control taking particular care to place it precisely across the narrowing in the bile duct. The delivery catheter is then removed leaving the stent standing as a scaffolding which holds the bile duct open. A further cholangiogram may be performed to document that the stent is appropriately positioned.

In certain embodiments, methods are provided for eliminating esophageal obstructions, comprising inserting an esophageal stent into an esophagus, the stent having a generally tubular structure, the surface of the structure being coated with (or otherwise adapted to release) an inventive compound or composition, such that the esophageal obstruction is eliminated. Briefly, the esophagus is the hollow tube which transports food and liquids from the mouth to the stomach. Cancer of the esophagus or invasion by cancer arising in adjacent organs (e.g., cancer of the stomach or lung) results in the inability to swallow food or saliva. In certain embodiments, a preinsertion examination, usually a barium swallow or endoscopy is performed in order to determine the appropriate position for stent insertion. A catheter or endoscope may then be positioned through the mouth, and a guidewire is advanced through the blockage. A stent delivery catheter is passed over the guidewire under radiologic or endoscopic control, and a stent is placed precisely across the narrowing in the esophagus. A post-insertion examination, usually a barium swallow x-ray, may be utilized to confirm appropriate positioning.

In certain embodiments, methods are provided for eliminating colonic obstructions, comprising inserting a colonic stent into a colon, the stent having a generally tubular structure, the surface of the structure being coated with (or otherwise adapted to release) an inventive compound or composition, such that the colonic obstruction is eliminated. Briefly, the colon is the hollow tube which transports digested food and waste materials from the small intestines to the anus. Cancer of the rectum and/or colon or invasion by cancer arising in adjacent organs (e.g., cancer of the uterus, ovary, bladder) results in the inability to eliminate feces from the bowel. In certain embodiments, a preinsertion examination, usually a barium enema or colonoscopy is performed in order to determine the appropriate position for stent insertion. A catheter or endoscope may then be positioned through the anus, and a guidewire is advanced through the blockage. A stent delivery catheter is passed over the guidewire under radiologic or endoscopic control, and a stent is placed precisely across the narrowing in the colon or rectum. A post-insertion examination, usually a barium enema x-ray, may be utilized to confirm appropriate positioning.

In certain embodiments, methods are provided for eliminating tracheal/bronchial obstructions, comprising inserting a tracheal/bronchial stent into a trachea or bronchi, the stent having a generally tubular structure, the surface of the structure being coated with (or otherwise adapted to release) an inventive compound or composition, such that the tracheal/bronchial obstruction is eliminated. Briefly, the trachea and bronchi are tubes which carry air from the mouth and nose to the lungs. Blockage of the trachea by cancer, invasion by cancer arising in adjacent organs (e.g., cancer of the lung), or collapse of the trachea or bronchi due to chondromalacia (weakening of the cartilage rings) results in inability to breathe. In certain embodiments, preinsertion examination, usually an endoscopy, is performed in order to determine the appropriate position for stent insertion. A catheter or endoscope is then positioned through the mouth, and a guidewire advanced through the blockage. A delivery catheter is then passed over the guidewire in order to allow a collapsed stent to be inserted. The stent is placed under radiologic or endoscopic control in order to place it precisely across the narrowing. The delivery catheter may then be removed leaving the stent standing as a scaffold on its own. A post-insertion examination, usually a bronchoscopy may be utilized to confirm appropriate positioning.

In certain embodiments, methods are provided for eliminating urethral obstructions, comprising inserting a urethral stent into a urethra, the stent having a generally tubular structure, the surface of the structure being coated with (or otherwise adapted to release) an inventive compound or composition, such that the urethral obstruction is eliminated. Briefly, the urethra is the tube which drains the bladder through the penis. Extrinsic narrowing of the urethra as it passes through the prostate, due to hypertrophy of the prostate, occurs in virtually every man over the age of 60 and causes progressive difficulty with urination. In certain embodiments, a preinsertion examination, usually an endoscopy or urethrogram is first performed in order to determine the appropriate position for stent insertion, which is above the external urinary sphincter at the lower end, and close to flush with the bladder neck at the upper end. An endoscope or catheter is then positioned through the penile opening and a guidewire advanced into the bladder. A delivery catheter is then passed over the guidewire in order to allow stent insertion. The delivery catheter is then removed, and the stent expanded into place. A post-insertion examination, usually endoscopy or retrograde urethrogram, may be utilized to confirm appropriate position.

In certain embodiments, methods are provided for eliminating vascular obstructions, comprising inserting a vascular stent into a blood vessel, the stent having a generally tubular structure, the surface of the structure being coated with (or otherwise adapted to release) an inventive compound or composition, such that the vascular obstruction is eliminated. Briefly, stents may be placed in a wide array of blood vessels, both arteries and veins, to prevent recurrent stenosis at the site of failed angioplasties, to treat narrowings that would likely fail if treated with angioplasty, and to treat post-surgical narrowings (e.g., dialysis graft stenosis). Suitable sites include, but are not limited to, the iliac, renal, and coronary arteries, the superior vena cava, and in dialysis grafts. In certain embodiments, angiography is first performed in order to localize the site for placement of the stent. This is typically accomplished by injecting radiopaque contrast through a catheter inserted into an artery or vein as an x-ray is taken. A catheter may then be inserted either percutaneously or by surgery into the femoral artery, brachial artery, femoral vein, or brachial vein, and advanced into the appropriate blood vessel by steering it through the vascular system under fluoroscopic guidance. A stent may then be positioned across the vascular stenosis. A post-insertion angiogram may also be utilized in order to confirm appropriate positioning.

Another aspect of the invention relates to a method for inhibiting the growth of multidrug resistant cells in a biological sample or a patient, which method comprises administering to the patient, or contacting said biological sample with a compound of formula I or a composition comprising said compound.

Another aspect of the invention relates to a method of treating or lessening the severity of a disease or condition associated with cell hyperproliferation in a patient, said method comprising a step of administering to said patient, a compound of formula I or a composition comprising said compound.

It will be appreciated that the compounds and compositions, according to the method of the present invention, may be administered using any amount and any route of administration effective for the treatment of cancer and/or disorders associated with cell hyperproliferation. For example, when using the inventive compounds for the treatment of cancer, the expression “effective amount” as used herein, refers to a sufficient amount of agent to inhibit cell proliferation, or refers to a sufficient amount to reduce the effects of cancer. The exact amount required will vary from subject to subject, depending on the species, age, and general condition of the subject, the severity of the diseases, the particular anticancer agent, its mode of administration, and the like.

The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being, treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety).

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, creams or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg /kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise specified, the contents of those cited references are incorporated herein by reference in their entirety to help illustrate the state of the art. Throughput this document, various publications are referred to, each of which is hereby incorporated by reference in its entirety in an effort to more fully describe the state of the art to which the invention pertains.

The following examples contain important additional information, exemplification and guidance that can be adapted to the practice of this invention in its various embodiments and the equivalents thereof.

### Exemplification

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

1) General Description of Synthetic Methods:

The practitioner has a a well-established literature of macrolide chemistry to draw upon, in combination with the information contained herein, for guidance on synthetic strategies, protecting groups, and other materials and methods useful for the synthesis of the compounds of this invention.

The various references cited herein provide helpful background information on preparing compounds similar to the inventive compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

According to the present invention, any available techniques can be used to make or prepare the inventive compounds or compositions including them. For example, a variety of solution phase synthetic methods such as those discussed in detail below may be used. Alternatively or additionally, the inventive compounds may be prepared using any of a variety combinatorial techniques, parallel synthesis and/or solid phase synthetic methods known in the art.

It will be appreciated as described below, that a variety of inventive compounds can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1990, “Comprehensive Organic Transformations: A Guide to Functional Group Preparations”, 2^{nd} ed. VCH Publishers. These schemes are merely illustrative of some methods by which the compounds of this invention can be synthesized, and various modifications to these schemes can be made and will be suggested to a person of ordinary skill in the art having regard to this disclosure.

The starting materials, intermediates, and compounds of this invention may be isolated and purified using conventional techniques, including filtration, distillation, crystallization, chromatography, and the like. They may be characterized using conventional methods, including physical constants and spectral data.

Certain exemplary compounds of the invention are listed below and are referred to by compound number as indicated.

| Structure | Compound |
|---|---|
| 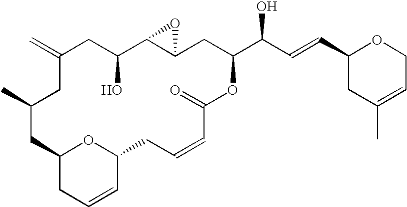 | ER-806782 |
| 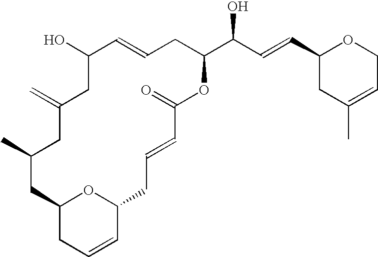 | ER-805883 |
| 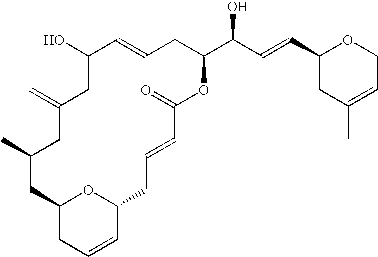 | ER-805884 |
| 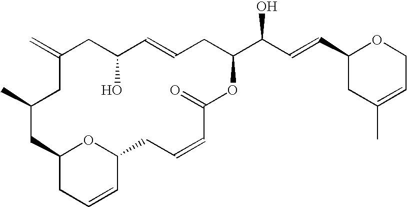 | ER-805885 |
| 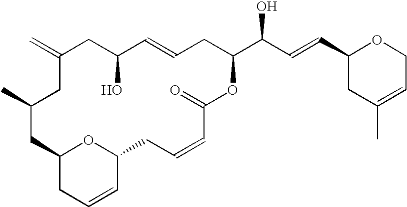 | ER-805886 |
| 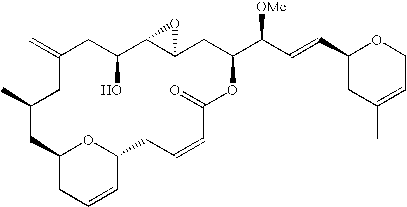 | ER-807397 |
| 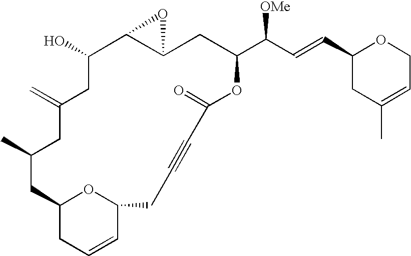 | ER-807398 |
| 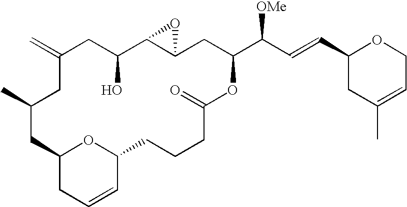 | ER-807308 |
| 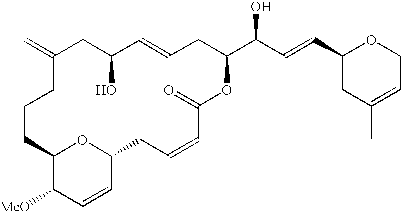 | ER-807127 |
| 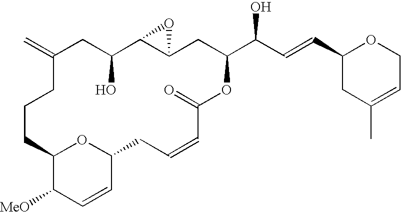 | ER-807331 |
| 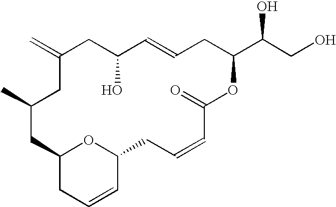 | ER-807129 |
| 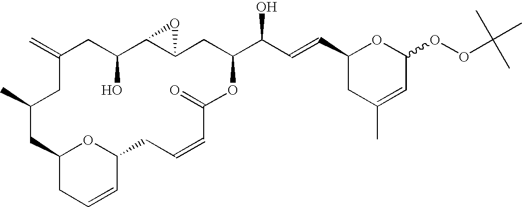 | ER-808455 |
| 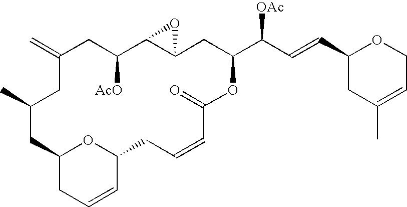 | ER-808545 |
| 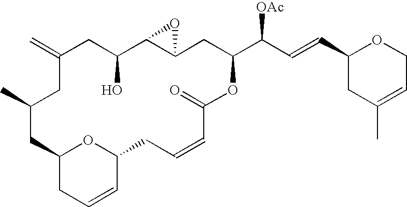 | ER-808546 |
| 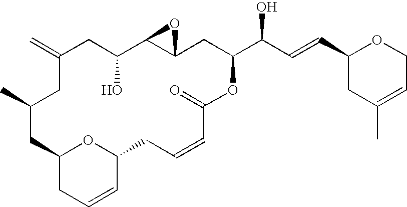 | ER-808547 |
| 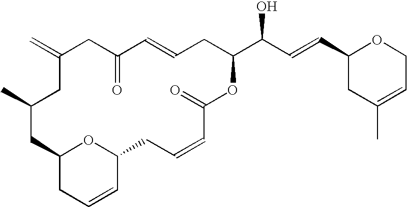 | ER-808550 |
| 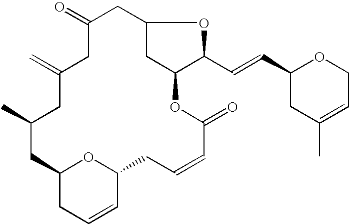 | ER-808551 |
| 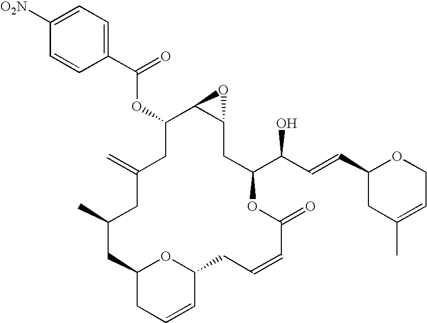 | ER-808572 |
| 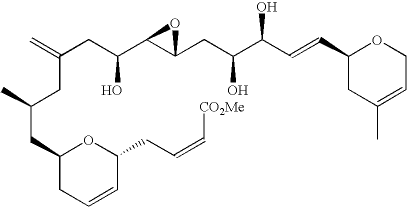 | ER-808573 |
| 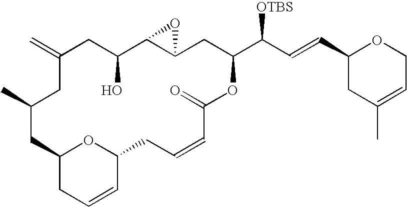 | ER-808574 |
| 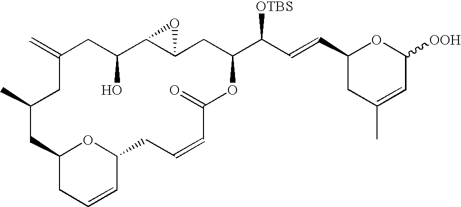 | ER-808575 |
| 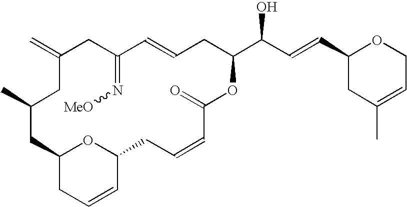 | ER-808626 |
| 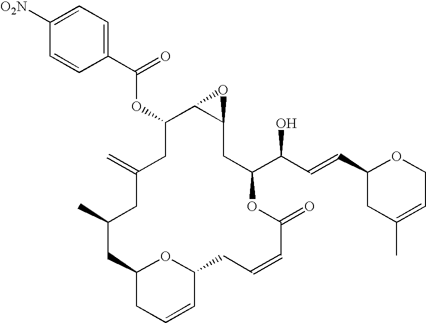 | ER-808715 |
| 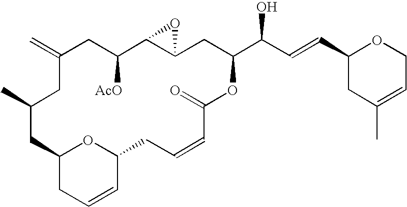 | ER-808716 |
| 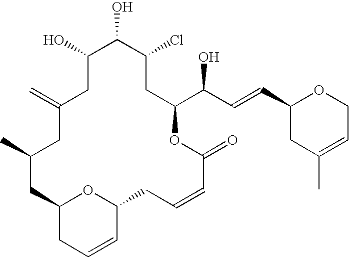 | ER-808859 |
| 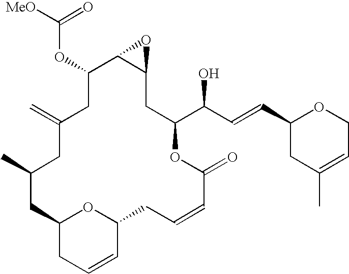 | ER-808860 |
| 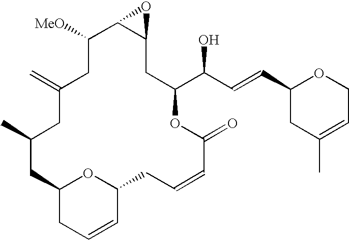 | ER-809170 |
| 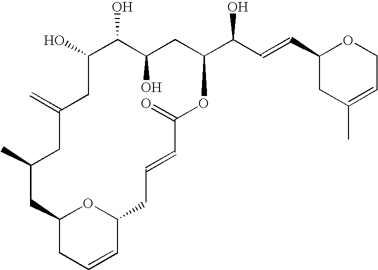 | ER-809171 |
| 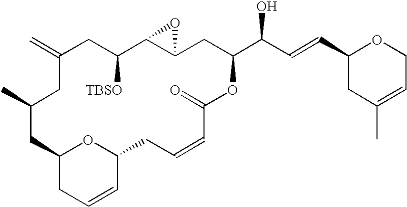 | ER-809172 |
| 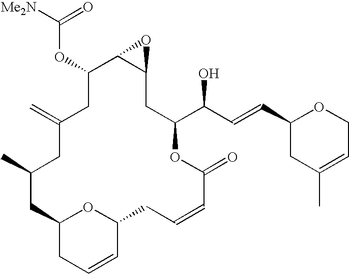 | ER-809173 |

General Reaction Procedures:

Unless mentioned specifically, reaction mixtures were stirred using a magnetically driven stirrer bar. An inert atmosphere refers to either dry argon or dry nitrogen. Reactions were monitored either by thin layer chromatography (TLC), by proton nuclear magnetic resonance (NMR) or by high-pressure liquid chromatography (HPLC), of a suitably worked up sample of the reaction mixture.

Listed below are abbreviations used for some common organic reagents referred to herein:

DDQ: 2,3-Dichloro-5,6-dicyano-1,4-benzoquinone

DEAD: Diethylazodicarboxykate

DIPT: Diisopropyl tartrate

DMAP: N,N-Dimethylaminopyridine

MOMCl: Methoxymethylchloride

PNBz: para-Nitrobenzoyl

RT: Room temperature

TBAF: Tetra n-butyl ammonium fluoride

TBS: Tri-butyl silyl

TBSOTf: Tert-butyl-dimethylsilyl triflate

TIPS: Tri-isopropyl silyl

THF: Tetrahydrofuran

General Work Up Procedures:

Unless mentioned specifically, reaction mixtures were cooled to room temperature or below then quenched, when necessary, with either water or a saturated aqueous solution of ammonium chloride. Desired products were extracted by partitioning between water and a suitable water-immiscible solvent (e.g. ethyl acetate, dichloromethane, diethyl ether). The desired product containing extracts were washed appropriately with water followed by a saturated solution of brine. On occasions where the product containing extract was deemed to contain residual oxidants, the extract was washed with a 10% solution of sodium sulphite in saturated aqueous sodium bicarbonate solution, prior to the aforementioned washing procedure. On occasions where the product containing extract was deemed to contain residual acids, the extract was washed with saturated aqueous sodium bicarbonate solution, prior to the aforementioned washing procedure (except in those cases where the desired product itself had acidic character). On occasions where the product containing extract was deemed to contain residual bases, the extract was washed with 10% aqueous citric acid solution, prior to the aforementioned washing procedure (except in those cases where the desired product itself had basic character). Post washing, the desired product containing extracts were dried over anhydrous magnesium sulphate, and then filtered. The crude products were then isolated by removal of solvent(s) by rotary evaporation under reduced pressure, at an appropriate temperature (generally less than 45° C.).

General Purification Procedures:

Unless mentioned specifically, chromatographic purification refers to flash column chromatography on silica, using a single solvent or mixed solvent as eluent. Suitably purified desired product containing elutes were combined and concentrated under reduced pressure at an appropriate temperature (generally less than 45° C.) to constant mass. Final compounds were dissolved in 50% aqueous acetonitrile or benzene, filtered and transferred to vials, then freeze-dried under high vacuum before submission for biological testing.

1) Synthesis of Exemplary Compounds:

Unless otherwise indicated, starting materials are either commercially available or readily accessibly through laboratory synthesis by anyone reasonably familiar with the art. Described generally below, are procedures and general guidance for the synthesis of compounds as described generally and in subclasses and species herein. In addition, synthetic guidance can be found in published U.S. patent application 2002/0128471, published PCT application WO 01/54689 and published European patent application EP 1295886, the entire contents of which are hereby incorporated by reference. Additional synthetic guidance may be found in Ghosh et al., 2001, “Total synthesis of microtubule-stabilizing agent (−)-Laulimalide,” J. Org. Chem. 66: 8973-8982; Paterson et al., 2001, “Total synthesis of microtubule-stabilizing agent (−)-Laulimalide,” Org. Lett.: 3149-3152; Enev et al., 2001, “Macrocyclization via allyl transfer: total synthesis of Laulimalide,” J. Am. Chem. Soc. 123: 10764-10765; Ghosh et al., 2000, “Total synthesis of (−)-Laulimalide,” J. Am. Chem. Soc. 122: 11027-11028 and references cited therein.

### Example 1

### Preparation of ER-806341

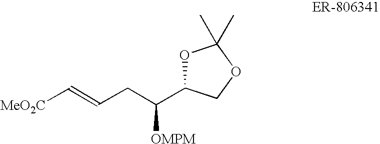

Exemplary Synthesis:

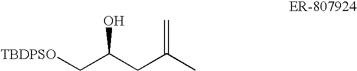

1-(tert-Butyl-diphenyl-silanyloxy)-4-methyl-pent-4-en-2-ol. R-Glycidol (10 g, 135 mmol), Et₃N (20.6 mL, 148 mmol), and DMAP (0.82 g, 6.71 mmol) were dissolved in CH₂Cl₂ (250 mL) and cooled to −50° C. TBDPSCl (37.1 mL, 39.2 g, 143 mmol) was added, and the mixture was stirred for 4 h then gradually warmed to RT and stirred overnight. Typical aqueous work up provided 45 g of crude TBDPS protected R-glycidol. The crude product (21 g, 67 mmol) was taken up in THF (500 ml), cooled to −50° C., CuI (12.8 g, 67.2 mmol) was added followed by dropwise addition of isopropenyl magnesium bromide (200 mL of a 0.5M solution in THF, 100 mmol). The reaction temperature was slowly increased to −20° C. over 1 h. Typical aqueous work up provided 30 g of the crude product. Spectral data confirmed the structure of the product.

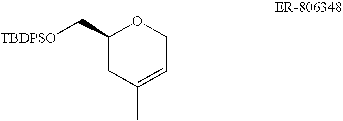

tert-Butyl-(4-methyl-3,6-dihydro-2H-pyran-2-ylmethoxy)-diphenyl-silane. The crude starting material (124 g, 350 mmol) and allyl bromide (121 mL, 169 g, 1.40 mol) were dissolved in THF (1 L), cooled to 5° C., and t-BuOK (455 mL of a 1M solution in THF, 455 mmol) was added slowly over 45 min. Typical aqueous work up provided crude allylated product. The crude material was taken into anhydrous CH₂Cl₂ (1 L), bis(tricyclohexylphosphine)benzylidene ruthenium (IV) dichloride (8.65 g, 10.46 mmol) was added, and the mixture was refluxed overnight. Concentration and chromatography provided the product. Spectral data confirmed the structure of the product.

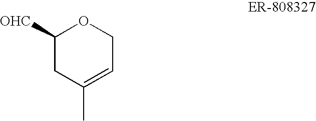

4-Methyl-3,6-dihydro-2H-pyran-2-carbaldehyde. The starting material (5.0 g, 14.3 mmol) was dissolved in THF (100 mL) and solid TBAF (5.35 g, 20.46 mmol) was added portionwise. The reaction was stirred at RT for 45 min. Typical aqueous work up and chromatography provided the crude alcohol. COCl₂ (1.77 mL of a 2M solution in CH₂Cl₂, 3.54 mmol) was dissolved in CH₂Cl₂ (14 mL), cooled to −78° C., DMSO (0.44 mL, 6.20 mmol) was added dropwise, followed by dropwise addition of the starting material. After 20 min, Et₃N was added and the reaction was allowed to warm to RT over 45 min. Typical aqueous workup provided the crude product which was used without purification. Spectral data confirmed the structure of the product.

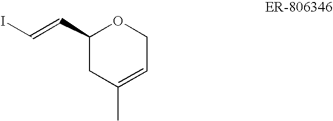

2-(2-Iodo-vinyl)-4-methyl-3,6-dihydro-2H-pyran. CrCl₂ (9.43 g, 76.73 mmol) was suspended in THF (120 mL), cooled to 0° C., and a solution of CHI₃ (10.45 g, 26.54 mmol) and the pyran aldehyde (1.68 g, 13.29 mmol) in THF (90 mL) was added dropwise. Typical aqueous workup and chromatography provided 1.31 mg of product. Spectral data confirmed the structure of the product.

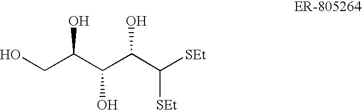

5,5-Bis-ethylsulfanyl-pentane-1,2,3,4-tetraol. D-Arabinose (305.67 g, 2.036 mol) and ZnCl₂ (50.06 g, 0.367 mol) were dissolved in concentrated hydrochloric acid (330 mL), cooled to ˜0° C., and EtSH (300 mL, 251.70 g, 4.051 mol) was added dropwise over a period of 45 min while maintaining the temperature near 0° C., then stirred for 45 min at ˜0° C. Typical aqueous workup provided 463.02 g of product as a white powder. Spectral data confirmed the structure of the product. MS (API, ESP+) m/z 279 [M+Na]⁺

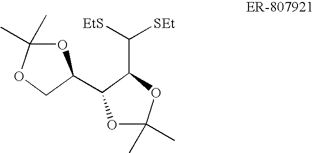

5-(Bis-ethylsulfanyl-methyl)-2,2,2′,2′-tetramethyl-[4,4′]bi[[1,3]dioxolanyl]. The thioacetal (410.72 g, 1.602 mol) was dissolved in acetone (3.3 L), cooled to ˜0° C., and P₂O₅ (310 g, 2.184 mol) was added in 5 portions. The mixture was warmed to room temperature and stirred ˜72 hours. Neutralization with sat. aq. NaHCO₃ and typical aqueous workup provided 399.35 g of crude product. Spectral data confirmed the structure of the product.

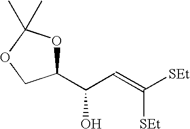

1-(2,2-Dimethyl-[1,3]dioxolan-4-yl)-3,3-bis-ethylsulfanyl-prop-2-en-1-ol. Solid t-BuOK (168.17 g, 1.499 mol), was dissolved in THF (2 L), and DMSO (700 mL). A solution of the bis-acetonide (399.30 g, 1.186 mol) in THF (700 mL) was added dropwise at RT over a period of ˜60 min. The resulting solution was stirred at RT for ˜1 h. Typical aqueous workup provided 292.20 g of crude product. Spectral data confirmed the structure of the product.

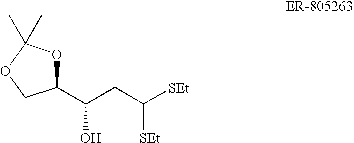

1-(2,2-Dimethyl-[1,3]dioxolan-4-yl)-3,3-bis-ethylsulfanyl-propan-1-ol. LiAlH₄ pellets (43.28 g, 1.140 mol) and were suspended in THF (2.3 L). The mixture was cooled to 0° C., and a solution of the acetonide (292.20 g, 1.049 mol) in THF (500 mL) was added dropwise over a period of ˜1 h while maintaining the temperature below ˜10° C. Upon completion of the reaction, typical aqueous workup provided 261.50 g of crude product. Spectral data confirmed the structure of the product.

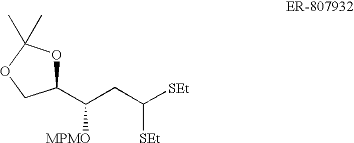

4-[3,3-Bis-ethylsulfanyl-1-(4-methoxy-benzyloxy)-propyl]-2,2-dimethyl-[1,3]dioxolane. NaH (18 g of a 60% dispersion in mineral oil, 0.44 mol) was suspended in DMF (2 L). A solution of the alcohol (95 g, 0.34 mol) in DMF (500 mL) was added dropwise, the resulting mixture was stirred for 30 min, then p-methoxybenzyl chloride (60 mL, 0.44 mol) was added dropwise. The reaction was stirred at RT for 4 h, then typical aqueous workup and chromatography provided 81 g of product. Spectral data confirmed the structure of the product. MS (API, ESP+) m/z 423 [M+Na]⁺

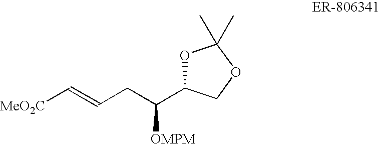

5-(2,2-Dimethyl-[1,3]dioxolan-4-yl)-5-(4-methoxy-benzyloxy)-pent-2-enoic acid methyl ester. A solution of the thioacetal (177 g, 0.44 mol) in acetone (3 L) was treated with solid NaHCO₃ (138 g, 1.64 mol) followed by water (1 L). Solid iodine (168 g, 0.66 mol) was then added portionwise. Upon completion of the reaction, typical aqueous workup provided 150 g of crude product. NaH (26.5 g of a 60% dispersion in mineral oil, 0.66 mol) was suspended in THF (6.6 L), cooled to 0° C., and trimethyl-phosphonoacetate (107 mL, 0.44 mol) was added dropwise. The resulting mixture was stirred for 1 h at 0° C., then a solution of the crude aldehyde (150 g, ˜0.44 mol) in THF (600 mL) was added dropwise over a period of 1 h. Typical aqueous workup and chromatography provided 105 g of product as a ˜4:1 mixture of isomers. A solution of the ester (125 g, 0.357 mol) in CH₃CN (1.2 L) was treated with tributylphosphine (27 mL, 0.11 mol) and the resulting mixture was stirred at 60° C. Concentration and chromatography provided 118 g of product. Spectral data confirmed the structure of the product as virtually exclusively trans. MS (API, ESP+) m/z 373 [M+Na]⁺

### Example 2

### Preparation of ER-807910

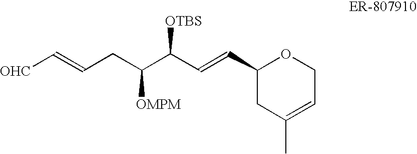

Exemplary Synthesis:

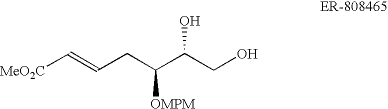

6,7-Dihydroxy-5-(4-methoxy-benzyloxy)-hept-2-enoic acid methyl ester. The acetonide (4.04 g, 11.54 mmol) was dissolved in THF (20 mL) and 1M aqueous HCl (20 mL) and stirred ˜12 h at RT. Typical aqueous workup and chromatography provided 2.96 g of product. Spectral data confirmed the structure of the product. MS (API, ESP+) m/z 333 [M+Na]⁺

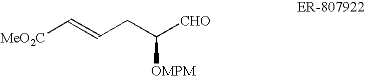

5-(4-Methoxy-benzyloxy)-6-oxo-hex-2-enoic acid methyl ester.

The diol (2.55 g, 8.22 mmol) was dissolved in THF (50 mL) and H₂O (50 mL), cooled to 0° C., and NaIO₄ (3.20 g, 14.94 mmol) was added. Typical aqueous workup provided 2.18 g of crude product. Spectral data continued the structure of the product.

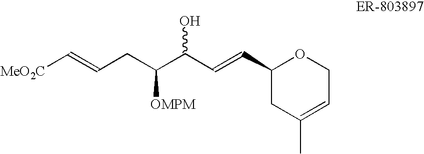

6-Hydroxy-5-(4-methoxy-benzyloxy)-8-(4-methyl-3,6-dihydro-2H-pyran-2-yl)-octa-2,7-dienoic acid methyl ester. The aldehyde (1.54 g, 5.53 mmol) and vinyl iodide (1.01 g, 4.04 mmol) were dissolved in degassed DMSO (20 mL). A mixture of 0.1% NiCl₂/CrCl₂ (1.41 g, 11.47 mmol) was added the reaction was stirred ˜36 h. The reaction was diluted with EtOAc (200 mL), stirred with Florisil (˜8 g) and Silica gel (˜8 g) for ˜1 h, then filtered. Concentration and chromatography yielded 1.36 g of product. Spectral data confirmed the structure of the product as a ˜1:2 mixture of diastereomers. MS (API, ESP+) m/z 425 [M+N]⁺

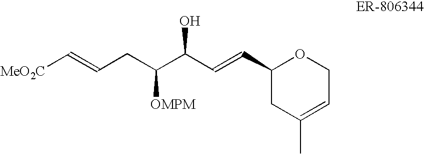

6-Hydroxy-5-(4-methoxy-benzyloxy)-8-(4-methyl-3,6-dihydro-2H-pyran-2-yl)-octa-2,7-dienoic acid methyl ester. The alcohol mixture (1.22 g, 3.02 mmol) was dissolved in CH₂Cl₂ (11 mL) and Dess-Martin periodane (1.93 g, 4.56 mmol) was added. Typical aqueous workup and chromatography provided 738 mg of enone. The enone was dissolved in THF (6 mL), cooled to −78° C., and L-Selectride (2.8 mL of a 1M solution in THF, 2.8 mmol) was added dropwise. Typical aqueous workup and chromatography provided 465 mg of product. Spectral data confirmed the structure of the product. MS (API, ESP+) m/z 425 [M+Na]⁺

Alternate procedure to 6-Hydroxy-5-(4-methoxy-benzyloxy)-8-(4-methyl-3,6-dihydro-2H-pyran-2-yl)-octa-2,7-dienoic acid methyl ester. DMSO (0.50 mL, 7.05 mmol) was dissolved in CH₂Cl₂ (10 mL), cooled to −78° C., and COCl₂ (2.1 mL of a 2M solution in CH₂Cl₂, 4.2 mmol) was added dropwise. After 20 min, a solution of alcohol SM (1.14 g, 2.83 mmol) in CH₂Cl₂ (15 mL) was added, stirred 15 min, then Et₃N (2.0 mL, 14.3 mmol) was added. Typical aqueous workup provided the crude aldehyde. The crude material was dissolved in THF (20 mL), cooled to −78° C., and L-Selectride (4.0 mL of a 1M solution in THF, 4.0 mmol) was added dropwise. Typical aqueous workup and chromatography provided 413 mg of product. Spectral data confirmed the structure of the product.

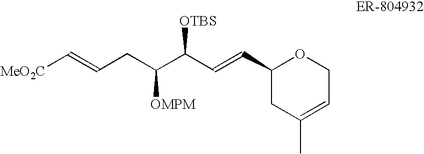

6-(tert-Butyl-dimethyl-silanyloxy)-5-(4-methoxy-benzyloxy)-8-(4-methyl-3,6-dihydro-2H-pyran-2-yl)-octa-2,7-dienoic acid methyl ester. The alcohol (413 mg, 1.02 mmol) and imidazole (0.22 g, 3.27 mmol) were dissolved in DMF (10 mL), cooled to 0° C., and TBSOTf (0.35 mL, 0.40 g, 1.52 mmol) was added dropwise. The reaction was warmed to RT and stirred overnight. Typical aqueous workup and chromatography provided 0.44 g of product. Spectral data confirmed the structure of the product. MS (API, ESP+) m/z 539 [M+Na]⁺

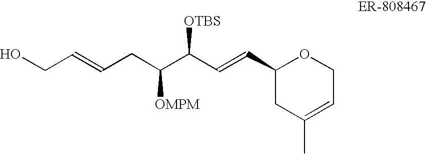

6-(tert-Butyl-dimethyl-silanyloxy)-5-(4-methoxy-benzyloxy)-8-(4-methyl-3,6-dihydro-2H-pyran-2-yl)-octa-2,7-dien-1-ol. The methyl ester (0.44 g, 0.85 mmol) was dissolved in CH₂Cl₂ (8.5 mL), cooled to −78° C., and DIBAL (2 mL of a 1M solution in CH₂Cl₂, 2 mmol) was added dropwise. Typical aqueous workup provided 0.40 g of crude product. Spectral data confirmed the structure of the product. MS (API, ESP+) m/z 509 [M+Na]⁺

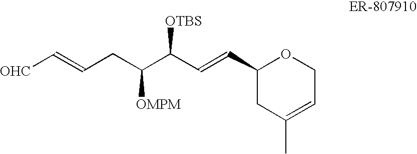

6-(tert-Butyl-dimethyl-silanyloxy)-5-(4-methoxy-benzyloxy)-8-(4-methyl-3,6-dihydro-2H-pyran-2-yl)-octa-2,7-dienal. DMSO (0.14 mL, 1.97 mmol) was dissolved in CH₂Cl₂ (3 mL), cooled to −78° C., and (COCl)₂ (0.53 mL of a 2M solution in CH₂Cl₂, 1.06 mmol) was added dropwise. After 20 min, a solution of the allylic alcohol (0.40 g, 0.82 mmol) in CH₂Cl₂ (5 mL) was added, stirred 15 min, then Et₃N (2.0 mL, 14.3 mmol) was added and the reaction was warmed to RT. Typical aqueous workup and chromatography provided the 336 mg of enal. Spectral data confirmed the structure of the product. MS (API, ESP+) m/z 511 [M+Na]⁺

Alternate procedure to 6-(tert-Butyl-dimethyl-silanyloxy)-5-(4-methoxy-benzyloxy)-8-(4-methyl-3,6-dihydro-2H-pyran-2-yl)-octa-2,7-dienal. Alcohol (0.14 g, 0.29 mmol) was dissolved in CH₂C₂ (2.5 mL), solid NaHCO₃ (0.51 g, 6.1 mmol) was added followed by Dess-Martin periodane (0.37 g, 0.88 mmol). Typical aqueous workup and chromatography provided 104 mg of product. Spectral data confirmed the structure of the product.

Alternate procedure to 6-(tert-Butyl-dimethyl-silanyloxy)-5-(4-methoxy-benzyloxy)-8-(4-methyl-3,6-dihydro-2H-pyran-2-yl)-octa-2,7-dienal. Alcohol (0.15 g, 0.30 mmol) was dissolved in CH₂Cl₂ (3 mL), cooled to 0° C., 4 Å molecular sieves (0.15 g), tetrapropylammonium perruthenate (14.5 mg, 0.014 mmol) and N-methylmorpholine N-oxide (51 mg, 0.44 mmol) were added. Filtration through Silica gel and concentration provided 126 mg of product. Spectral data confirmed the structure of the product.

### Example 3

### Alternate Horner-Wadsworth-Emmons Procedure for the Preparation of ER-807910

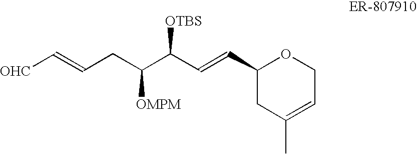

Exemplary Synthesis:

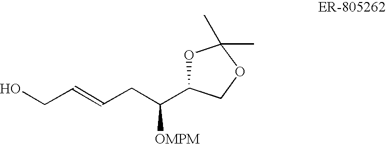

5-(2,2-Dimethyl-[1,3]dioxolan-4-yl)-5-(4-methoxy-benzyloxy)-pent-2-en-1-ol. The methyl ester (163 g, 467 mmol) was dissolved in CH₂Cl₂ (2.3 L), cooled to −78° C., and DIBAL (1.17 L of a 1M solution in CH₂Cl₂, 1.17 mol) was added dropwise. Typical aqueous workup provided 140 g of crude product. Spectral data confirmed the structure of the product.

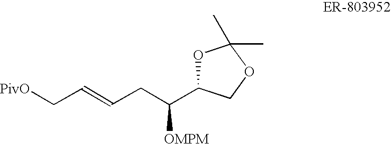

2,2-Dimethyl-propionic acid 5-(2,2-dimethyl-[1,3]dioxolan-4-yl)-5-(4-methoxy-benzyloxy)-pent-2-enyl ester. The allyl alcohol (140 g, 434 mmol), pyridine (110 mL, 1.35 mol), and DMAP (5.3 g, 43 mmol) were dissolved in CH₂Cl₂ (2 L) and pivaloyl chloride (67 mL, 543 mmol) was added dropwise. Typical aqueous workup and chromatography provided 155 g of product. Spectral data confirmed the structure of the product.

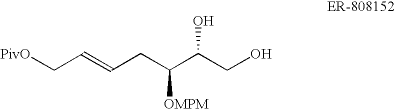

2,2-Dimethyl-propionic acid 6,7-dihydroxy-5-(4-methoxy-benzyloxy)-hept-2-enyl ester. The allyl pivaloate (92.6 g, 228 mmol) was dissolved in THF (500 mL) and 2.5M aqueous HCl (500 mL) at 0° C., then warmed to RT. Typical aqueous workup provided 79 g of product. Spectral data confirmed the structure of the product.

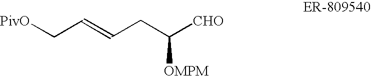

2,2-Dimethyl-propionic acid 5-(4-methoxy-benzyloxy)-6-oxo-hex-2-enyl ester. The diol (59 g, 0.162 mol) was dissolved in a 1/1 mixture THF/H₂O (3.2 L), cooled to 0° C., and sodium periodate (87.6 g, 0.40 mol) was added portion-wise. Filtration and typical aqueous workup gave 56 g of the desired aldehyde.

Spectral data confirmed the structure of the product. MS (API, ESP+) m/z 357 [M+Na]⁺

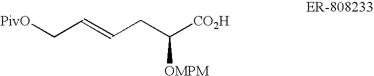

6-(2,2-Dimethyl-propionyloxy)-2-(4-methoxy-benzyloxy)-hex-4-enoic acid. The aldehyde (56 g, 163 mmol) was dissolved in t-BuOH (450 mL), cooled to 0° C., a solution of sulfamic acid (20.5 g, 212 mmol) in H₂O (450 mL) was added followed by dropwise addition of a solution of sodium chlorite (27.6 g, 245 mmol) in H₂O (450 mL). Typical aqueous workup provided 56.8 g of product. Spectral data confirmed the structure of the product. MS (API, ESP+) m/z 373 [M+Na]⁺

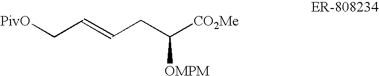

6-(2,2-Dimethyl-propionyloxy)-2-(4-methoxy-benzyloxy)-hex-4-enoic acid methyl ester. The acid (56.8 g, 162 mmol) was dissolved in toluene (630 mL) and MeOH (180 mL) and a solution of TMSCHN₂ (120 mL of a 2M solution in hexanes, 240 mmol) was added dropwise. Concentration and chromatography yielded 33 g of product. Spectral data confirmed the structure of the product. MS (API, ESP+) m/z 387 [M+Na]⁺

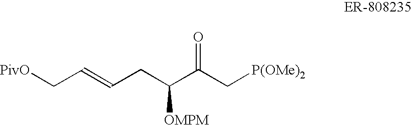

2,2-Dimethyl-propionic acid 7-(dimethoxy-phosphoryl)-5-(4-methoxy-benzyloxy)-6-oxo-hept-2-enyl ester. Dimethyl methylphosphonate (29.5 mL, 273 mmol) was dissolved in 550 mL of dry THF and cooled to −78° C. A 2.5 M of n-butyllithium in hexanes (107 mL, 267 mmol) was added dropwise and the reaction mixture was stirred at −78° C. for 15 min. A solution of the methyl ester (36.7 g, 101 mmol) in 110 mL of dry THF was added to the mixture, which was then stirred for 5 min. MeOH was added and the mixture was warmed slowly to RT. A solution of sodium methoxide 25 wt %) in MeOH (680 mL) was added to complete the cleavage of the pivaloyl group. Standard acidic workup and chromatography gave 35.6 g of the corresponding allylic alcohol. This material (35.6 g, 92 mmol), pyridine (22 mL, 276 mmol), and DMAP (1.1 g, 9 mmol) were dissolved in CH₂Cl₂ (460 mL) and pivaloyl chloride (14.2 mL, 115 mmol) was added dropwise. The reaction mixture was stirred for 16 h at RT. Typical aqueous workup and chromatography provided 155 g of product. Spectral data confirmed the structure of the product. MS (API, ESP+) m/z 479 [M+Na]⁺.

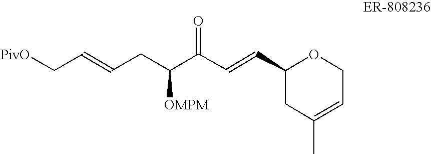

2,2-Dimethyl-propionic acid 5-(4-methoxy-benzyloxy)-8-(4-methyl-3,6-dihydro-2H-pyran-2-yl)-6-oxo-octa-2,7-dienyl ester. The phosphonate (28.5 g, 62.4 mmol) was dissolved in THF (312 mL), cooled to 0° C., and LiCl (6.56 g, 156 mmol) and triethylamine (21.7 mL, 15.8 g, 155 mmol) were added. After 10 min, a solution of aldehyde (11 g, 94 mmol) in THF (30 mL) was added dropwise, and the reaction was warmed to RT. Typical aqueous workup and chromatography yielded 19.6 g of product. Spectral data confirmed the structure of the product. MS (API, ESP+) m/z 479 [M+Na]⁺

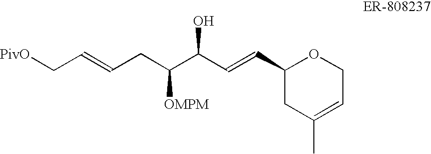

2,2-Dimethyl-propionic acid 6-hydroxy-5-(4-methoxy-benzyloxy)-8-(4-methyl-3,6-dihydro-2H-pyran-2-yl)-octa-2,7-dienyl ester. The enone (19.5 g, 42.7 mmol) was dissolved in THF (215 mL), cooled to −78° C., and a solution of L-Selectride (64 mL of a 1M solution in THF, 64 mmol) was added dropwise. Typical aqueous workup and chromatography provided 13.2 g of product. Spectral data confirmed the structure of the product. MS (API, ESP+) m/z 481 [M+Na]⁺

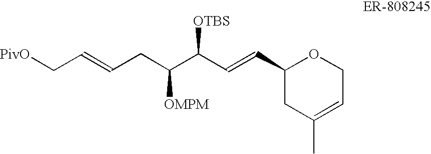

2,2-Dimethyl-propionic acid 6-(tert-butyl-dimethyl-silanyloxy)-5-(4-methoxy-benzyloxy)-8-(4-methyl-3,6-dihydro-2H-pyran-2-yl)-octa-2,7-dienyl ester. The allylic alcohol (13.6 g, 29.6 mmol) was dissolved in DMF (150 mL), cooled to 0° C., imidazole (10 g, 148 mmol) was added followed by TBSOTf (17 mL, 19.6 g, 74 mmol) and the misture was warmed to RT. Typical aqueous workup and chromatography provided 12 g of product. Spectral data confirmed the structure of the product. MS (API, ESP+) m/z 597 [M+Na]⁺

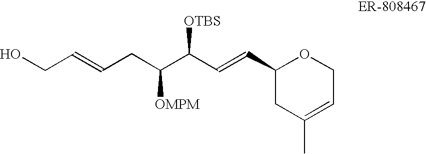

6-(tert-Butyl-dimethyl-silanyloxy)-5-(4-methoxy-benzyloxy)-8-(4-methyl-3,6-dihydro-2H-pyran-2-yl)-octa-2,7-dien-1-ol. The pivaloate (16.4 g, 28.6 mmol) was dissolved in MeOH (120 mL), cooled to 0° C., a solution of NaOMe (62 mL of a 25% wt solution in MeOH, 286 mmol) was added, and the reaction was warmed to RT. Typical aqueous workup provided 13.4 g of crude product. Spectral data confirmed the structure of the product. MS (API, ESP+) m/z 511 [M+Na]⁺. Swern oxidation would afford ER-807910.

### Example 4

### Preparation of ER-806836

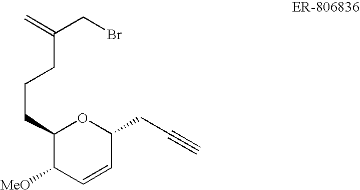

Exemplary Synthesis:

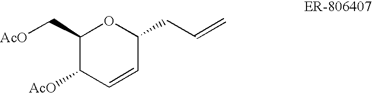

Acetic acid 2-acetoxymethyl-6-allyl-3,6-dihydro-2H-pyran-3-yl ester. Triacetyl glucal (223 g, 819 mmol) and trimethylallyl silane (200 g, 1.75 mol) were dissolved in CH₃CN (1.5 L), cooled to 0° C., TFA (64 mL, 95 g, 831 mmol) was added dropwise, and the reaction was warmed to RT. Typical aqueous workup provided 205 g of product. Spectral data confirmed the structure of the product.

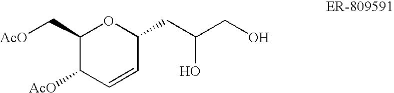

Acetic acid 3-acetoxy-6-(2,3-dihydroxy-propyl)-3,6-dihydro-2H-pyran-2-ylmethyl ester. The glycal (102 g, 400 mmol) was dissolved in THF (500 mL) and H₂O (250 mL), cooled to 0° C., K₂OsO₄-2H₂O (737 mg, 2.0 mmol) and NMO (47.4 g, 404 mmol) were added, and the reaction was warmed to RT. Typical aqueous workup yielded 107 g of product. Spectral data continued the structure of the product.

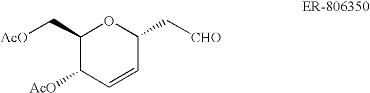

Acetic acid 2-acetoxymethyl-6-(2-oxo-ethyl)-3,6-dihydro-2H-pyran-3-yl ester. The diol (107 g, 371 mmol) was dissolved in CH₂Cl₂ (1 L) and sat. aq. NaHCO₃ (60 mL) and NaIO₄ (58 g, 271 mmol) was added. Typical aqueous workup produced 71.6 g of product. Spectral data confirmed the structure of the product.

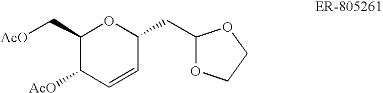

Acetic acid 2-acetoxymethyl-6-[1,3]dioxolan-2-ylmethyl-3,6-dihydro-2H-pyran-3-yl ester. The aldehyde (33.5 g, 131 mmol), p-TsOH—H₂O (1.2 g, 6.5 mmol), and ethylene glycol (33 mL, 37 g, 592 mmol) were dissolved in benzene (350 mL). The flask was equipped with a pressure equilibrating dropping funnel packed with 4 Å molecular sieves and CaSO₄ (drierite) and topped with a condenser. The reaction was refluxed for 1.5 h. Typical aqueous workup and recrystallization (MTBE) produced 29.1 g of product. Spectral data confirmed the structure of the product.

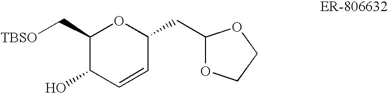

2-(tert-Butyl-dimethyl-silanyloxymethyl)-6-[1,3]dioxolan-2-ylmethyl-3,6-dihydro-2H-pyran-3-ol. The bis-acetate was hydrolyzed to the diol in a similar manner to ER-809594. The diol (1.0 g, 4.7 mmol) was dissolved in DMF (10 mL), imidazole (0.47 g, 6.90 mmol) was added, the reaction cooled to ˜10° C., and TBSCl (0.74 g. 4.91 mmol) was added. Typical aqueous workup provided 1.31 g of crude product. Spectral data confirmed the structure of the product.

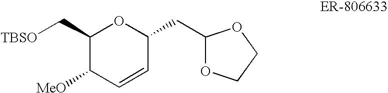

tert-Butyl-(6-[1,3]dioxolan-2-ylmethyl-3-methoxy-3,6-dihydro-2H-pyran-2-ylmethoxy)-dimethyl-silane. The alcohol (0.73 g, 2.21 mmol) was dissolved in DMF (10 mL), MeI (0.21 mL, 0.48 g, 3.37 mmol) was added, the reaction cooled to 0° C., NaH (0.11 g of a 60% suspension in mineral oil, 2.75 mmol) was added, and the reaction was allowed to warm to RT. Typical aqueous workup provided 0.70 g of crude product. Spectral data confirmed the structure of the product.

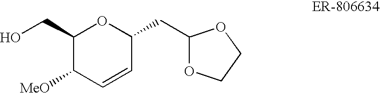

(6-[1,3]Dioxolan-2-ylmethyl-3-methoxy-3,6-dihydro-2H-pyran-2-yl)-methanol. The methyl ether (0.70 g, 2.03 mmol) was dissolved in THF (5 mL) and TBAF (6 mL of a 1M solution in THF, 6 mmol) was added. Typical aqueous workup and chromatography provided 0.50 g of crude product. Spectral data confirmed the structure of the product.

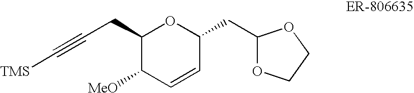

[3-(6-[1,3]Dioxolan-2-ylmethyl-3-methoxy-3,6-dihydro-2H-pyran-2-yl)-prop-1-ynyl]-trimethyl-silane. The alcohol (1.0 g, 4.3 mmol) was dissolved in CH₂Cl₂ (20 mL), cooled to −50° C., 2,6-di-t-butylpyridine (1.36 mL, 1.16 g, 6.07 mmol) was added followed by dropwise addition of Tf₂O (0.88 mL, 1.48 g, 5.23 mmol). Typical aqueous workup provided the crude triflate. The crude material was dissolved in THF (20 mL) and added dropwise to a cold (−78° C.) solution of TMS lithium acetylide (35 mL of a 0.5M solution in THF, 17.5 mmol) and HMPA (3.0 mL, 3.1 g, 17.2 mmol). Typical aqueous workup and chromatography provided 1.08 g of product. Spectral data confirmed the structure of the product.

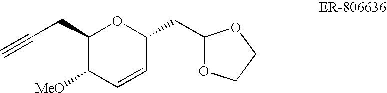

6-[1,3]Dioxolan-2-ylmethyl-3-methoxy-2-prop-2-ynyl-3,6-dihydro-2H-pyran. The TMS acetylide (0.50 g, 1.60 mmol) and Cs₂CO₃ (78 mg, 0.24 mmol) were dissolved in MeOH (10 mL). Typical aqueous workup provided 0.38 g of product. Spectral data confirmed the structure of the product.

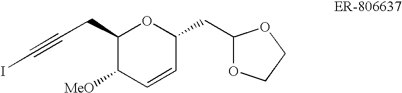

6-[1,3]Dioxolan-2-ylmethyl-2-(3-iodo-prop-2-ynyl)-3-methoxy-3,6-dihydro-2H-pyran. The alkyne (0.38 g, 1.60 mmol) was dissolved in acetone (10 mL), and AgNO₃ (0.30 g of a 10% w/w on Silica) and NIS (0.44 g, 1.96 mmol) were added. Typical aqueous workup and chromatography yielded 0.41 g of product. Spectral data confirmed the structure of the product. MS (API, ESP+) m/z 387 [M+Na]⁺.

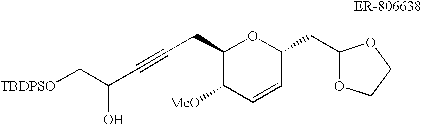

1-(tert-Butyl-diphenyl-silanyloxy)-5-(6-[1,3]dioxolan-2-ylmethyl-3-methoxy-3,6-dihydro-2H-pyran-2-yl)-pent-3-yn-2-ol. The alkynyl iodide (1.70 g, 4.70 mmol) and aldehyde (2.0 g, 6.7 mmol; for preparation, see e.g. W.-B. Choi et al. J. Am. Chem. Soc. 1991, 113, 9377) were dissolved in THF (50 mL), the solution was degassed (flow of N₂), and a mixture of 0.1% NiCl₂/CrCl₂ (4.3 g, 35.0 mmol) was added. Typical aqueous workup and chromatography produced 2.23 g of product. Spectral data confirmed the structure of the product.

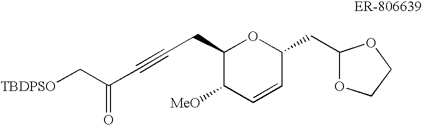

1-(tert-Butyl-diphenyl-silanyloxy)-5-(6-[1,3]dioxolan-2-ylmethyl-3-methoxy-3,6-dihydro-2H-pyran-2-yl)-pent-3-yn-2-one. The alcohol (50 mg, 0.09 mmol), NMO (16 mg, 0.14 mmol) and 4 Å molecular sieves (51 mg) were dissolved in CH₂Cl₂ (1 mL) and tetrapropylammonium perruthenate (2.7 mg, 0.008 mmol) was added. Typical aqueous workup yielded 48 mg of crude product. Spectral data confirmed the structure of the product.

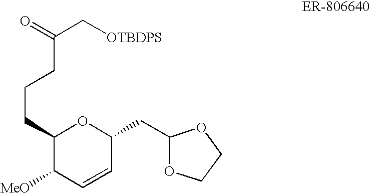

1-(tert-Butyl-diphenyl-silanyloxy)-5-(6-[1,3]dioxolan-2-ylmethyl-3-methoxy-3,6-dihydro-2H-pyran-2-yl)-pentan-2-one. The ynone (2.10 g, 3.90 mmol) was dissolved in toluene (60 mL) and H₂O (0.16 mL), the solution was degassed (flow of N₂), and Stryker's reagent (4.61 g, 2.35 mmol) was added. Typical aqueous workup and chromatography produced 1.33 g of product. Spectral data confirmed the structure of the product.

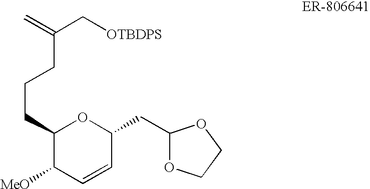

tert-Butyl-[5-(6-[1,3]dioxolan-2-ylmethyl-3-methoxy-3,6-dihydro-2H-pyran-2-yl)-2-methylene-pentyloxy]-diphenyl-silane. Methyl triphenylphosphonium bromide (3.32 g, 6.18 mmol) was added portionwise to a cool (0° C.) solution of n-BuLi (3.9 mL of a 1.6M solution in hexanes, 6.2 mmol) in THF (10 mL). The mixture was warmed to RT for ˜30 min, then cooled to 0° C., and a solution of ketone (1.33 g, 2.47 mmol) in THF (15 mL) was added. Typical aqueous workup and chromatography produced 1.20 g of product. Spectral data confirmed the structure of the product. MS (API, ESP+) m/z 559 [M+Na]⁺.

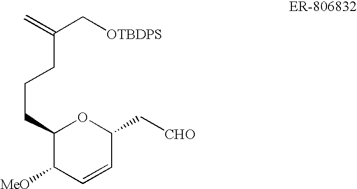

6-[4-(tert-Butyl-diphenyl-silanyloxymethyl)-pent-4-enyl]-5-methoxy-5,6-dihydro-2H-pyran-2-yl}-acetaldehyde. The dioxolane (480 mg, 0.89 mmol) was dissolved in 80% aqueous HOAc (20 mL) and heated to ˜60° C. Typical aqueous workup and chromatography provided 200 mg of product. Spectral data confirmed the structure of the product.

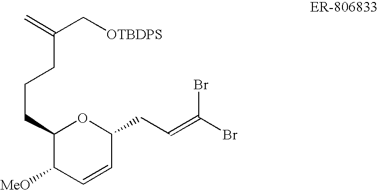

tert-Butyl-{5-[6-(3,3-dibromo-allyl)-3-methoxy-3,6-dihydro-2H-pyran-2-yl]-2-methylene-pentyloxy}-diphenyl-silane. A stock solution was prepared as follows: PPh₃ (0.63 g, 2.40 mmol) was dissolved in CH₂Cl₂ (1.5 mL), cooled to 0° C., and a solution of CBr₄ (0.40 g, 1.21 mmol) in CH₂Cl₂ (1.5 mL) was added and the mixture was allowed to stir at 0° C. for 10 min and 20 min at RT. A fraction of the stock solution (0.15 mL) was added to a cold (˜78° C.) solution of the aldehyde (9 mg, 0.02 mol) in CH₂Cl₂ (0.5 mL). Typical aqueous workup and preparative TLC produced 10.3 mg of product. Spectral data confirmed the structure of the product.

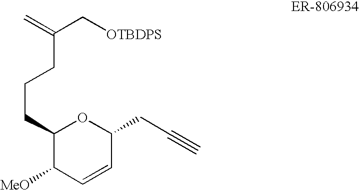

tert-Butyl-[5-(3-methoxy-6-prop-2-ynyl-3,6-dihydro-2H-pyran-2-yl)-2-methylene-pentyloxy]-diphenyl-silane. The di-bromide (250 mg, 0.39 mmol) was dissolved in THF (6 mL), cooled to −78° C., and n-BuLi (0.73 mL of a 1.6M solution in hexanes, 1.2 mmol) was added dropwise. Typical aqueous workup produced 187 mg of crude product. Spectral data confirmed the structure of the product.

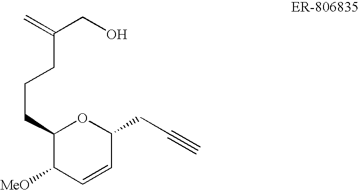

5-(3-Methoxy-6-prop-2-ynyl-3,6-dihydro-2H-pyran-2-yl)-2-methylene-pentan-1-ol. The alkyne (7.8 mg, 0.016 mmol) and TBAF (0.19 mL of a 1M solution in THF, 0.19 mmol) were dissolved in THF (0.5 mL). Typical aqueous workup and chromatography yielded 4 mg of product. Spectral data confirmed the structure of the product.

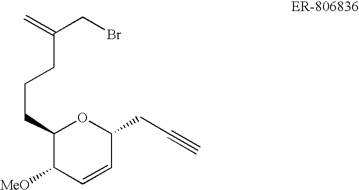

2-(4-Bromomethyl-pent-4-enyl)-3-methoxy-6-prop-2-ynyl-3,6-dihydro-2H-pyran. The alcohol (90 mg, 0.36 mmol) was dissolved in CH₂Cl₂ (1.5 mL), cooled to 0° C., and Ph₃P (123 mg, 0.47 mmol) and NBS (90 mg, 0.51 mmol) were added. Typical aqueous workup and chromatography produced 104 mg of product. Spectral data confirmed the structure of the product.

### Example 5

### Coupling of ER-806836 and ER-807910 and Preparation of Laulimalide Analogues

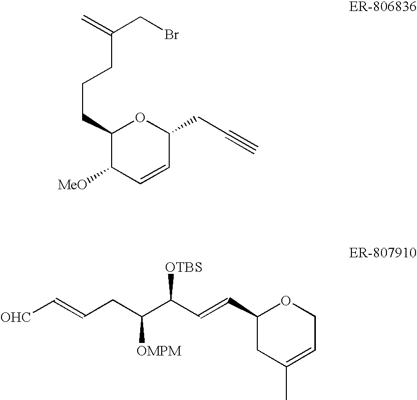

Exemplary Synthesis:

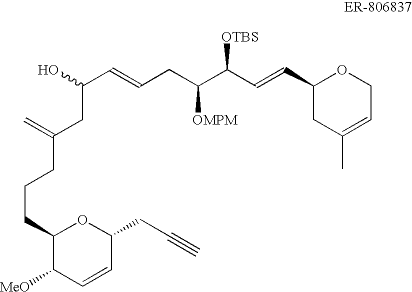

11-(tert-Butyl-dimethyl-silanyloxy)-10-(4-methoxy-benzyloxy)-1-(3-methoxy-6-prop-2-ynyl-3,6-dihydro-2H-pyran-2-yl)-13-(4-methyl-3,6-dihydro-2H-pyran-2-yl)-4-methylene-trideca-7,12-dien-6-ol. The bromide (30 mg, 0.096 mmol) and enal (70 mg, 0.14 mmol) were dissolved in THF (3 mL) and H₂O (1 mL), then Indium powder (44 mg, 0.38 mmol) and 0.2M aq. HCl (88 μL) were added. Filtration, typical aqueous workup, and chromatography produced 43 mg of product. Spectral data confirmed the structure of the product. MS (API, ESP+) m/z 743 [M+N]⁺.

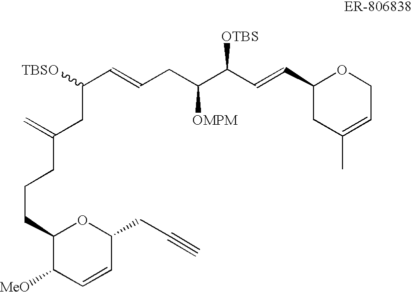

2-[6,11-Bis-(tert-butyl-dimethyl-silanyloxy)-10-(4-methoxy-benzyloxy)-13-(4-methyl-3,6-dihydro-2H-pyran-2-yl)-4-methylene-trideca-7,12-dienyl]-3-methoxy-6-prop-2-ynyl-3,6-dihydro-2H-pyran. The alcohol (43 mg, 0.060 mmol) and DMAP (several crystals) were dissolved in pyridine (1 mL) and TBSOTf (5 drops) was added. Typical aqueous workup and chromatography yielded 40 mg of product. Spectral data confirmed the structure of the product.

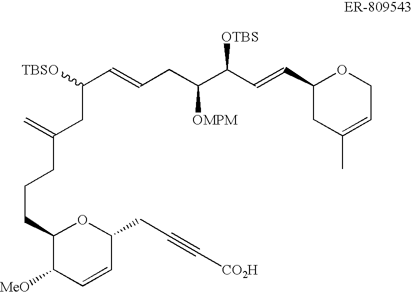

4-{6-[6,11-Bis-(tert-butyl-dimethyl-silanyloxy)-10-(4-methoxy-benzyloxy)-13-(4-methyl-3,6-dihydro-2H-pyran-2-yl)-4-methylene-trideca-7,12-dienyl]-5-methoxy-5,6-dihydro-2H-pyran-2-yl}-but-2-ynoic acid. The alkyne (40 mg, 0.048 mmol) was dissolved in THF (0.3 mL), cooled to −78° C., and n-BuLi (0.18 mL of a 1.6M solution in hexanes, 0.29 mmol) was added. After ˜5 min, a stream of CO₂ gas was bubbled through the reaction mixture. Typical aqueous workup and chromatography provided 24 mg of product. Spectral data confirmed the structure of the product.

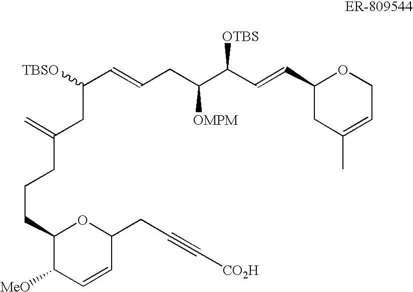

4-{6-[6,11-Bis-(tert-butyl-dimethyl-silanyloxy)-10-hydroxy-13-(4-methyl-3,6-dihydro-2H-pyran-2-yl)-4-methylene-trideca-7,12-dienyl]-5-methoxy-5,6-dihydro-2H-pyran-2-yl}-but-2-ynoic acid. The acid (24 mg, 0.027 mmol) and DDQ (25 mg, 0.11 mmol) were dissolved in a 2/1 mixture of CH₂Cl₂/H₂O (0.6 mL). Typical aqueous workup and chromatography provided 26 mg of product. Spectral data confirmed the structure of the product. MS (API, ESP−) m/z 878 [M−H]⁻.

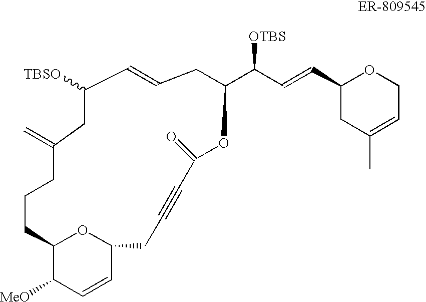

11-(tert-Butyl-dimethyl-silanyloxy)-7-[1-(tert-butyl-dimethyl-silanyloxy)-3-(4-methyl-3,6-dihydro-2H-pyran-2-yl)-allyl]-18-methoxy-13-methylene-6,21-dioxa-bicyclo[15.3.1]henicosa-9,19-dien-3-yn-5-one. The acid (5.0 mg, 0.0066 mmol) was dissolved in THF (0.4 mL), i-Pr₂NEt (2.3 μL, 1.7 mg, 0.013 mmol) was added followed by trichlorobenzoyl chloride (1.7 μL, 2.7 mg, 0.011 mmol). The reaction was stirred for 2 h then diluted with toluene (3 mL) and added 111 via syringe pump (6 mL/h rate) to a solution of DMAP (12 mg, 0.098 mmol) in toluene (3 mL) at 45° C. Typical aqueous workup and chromatography produced 2.0 mg of product. Spectral data confirmed the structure of the product. MS (API, ESP+) m/z 763 [M+Na]⁺.

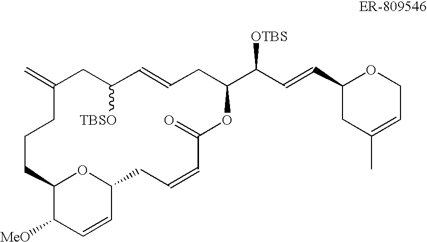

11-(tert-Butyl-dimethyl-silanyloxy)-7-[1-(tert-butyl-dimethyl-silanyloxy)-3-(4-methyl-3,6-dihydro-2H-pyran-2-yl)-allyl]-18-methoxy-13-methylene-6,21-dioxa-bicyclo[15.3.1]henicosa-3,9,19-trien-5-one. The alkynyl macrocycle (5.0 mg, 0.0067 mmol) was dissolved in a 3/1 mixture of hexanes/CH₂Cl₂ (2 mL), quinoline (3.2 μL, 3.5 mg, 0.027 mmol) was added followed by Lindlar catalyst (5 mg). The mixture was subjected to three vacuum/purge cycles and then stirred under H₂ (balloon). Filtration, concentration, and chromatography produced 4 mg of product. Spectral data confirmed the structure of the product. MS (API, ESP+) m/z 765 [M+H]⁺.

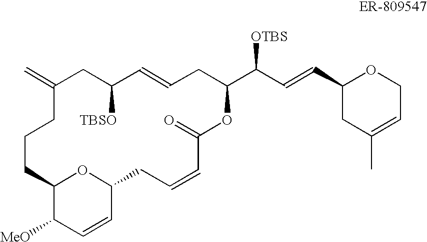

HPLC purification of the C.15 diastereomers. The mixture of diastereomers was separated by semi-prep HPLC using Chiralpak AD semi-prep column and a 1% IPA/hexanes mobile phase.

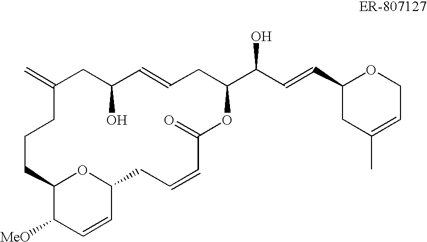

11-Hydroxy-7-[1-hydroxy-3-(4-methyl-3,6-dihydro-2H-pyran-2-yl)-ally]-18-methoxy-13-methylene-6,21-dioxa-bicyclo[15.3.1]henicosa-3,9,19-trien-5-one. The bis-TBS compound (1.8 mg, 0.0024 mmol) was dissolved in CH₃CN (0.2 mL) and H₂SiF₆ (1 drop of a 20-25% wt aqueous solution) was added. Typical aqueous workup and chromatography produced 1.2 mg of product. Spectral data confirmed the structure of the product. MS (API, ESP+) m/z 537 [M+Na]⁺.

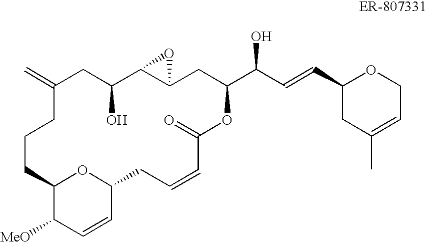

7-Hydroxy-12-[1-hydroxy-3-(4-methyl-3,6-dihydro-2H-pyran-2-yl)-allyl]-21-methoxy-5-methylene-9,13,22-trioxa-tricyclo[16.3.1.08,10]docosa-15,19-dien-14-one. A stock solution was made as follows: (+)-DIPT (87 μL, 97 mg, 0.41 mmol) was dissolved in CH₂Cl₂ (1 mL), cooled to −78° C., Ti(OiPr)₄ (98 μL, 94 mg, 0.33 mmol) was added, the mixture stirred 5 min, then t-BuOOH (90 μL of a ˜5.5M solution in nonane, 0.50 mmol) was added and the solution stirred for an additional 15 min. The diol (1.4 mg, 0.0027 mmol) was dissolved in CH₂Cl₂ (0.3 mL), cooled to −15° C., and portions of the stock solution were added until the reaction was complete. Typical aqueous workup and chromatography produced 0.5 mg of product. Spectral data confirmed the structure of the product. MS (API, ESP+) m/z 753 [M+Na]⁺.

### Example 6

### Preparation of ER-807321

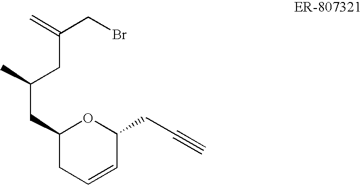

Exemplary Synthesis:

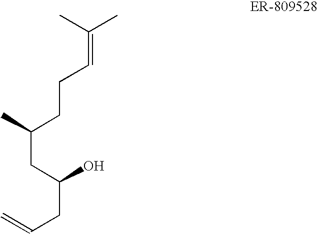

6,10-Dimethyl-undeca-1,9-dien-4-ol. (−)-B-Methoxydiisopinocampheylborane (58 g, 183 mmol) was dissolved in diethyl ether (200 mL), cooled to 0° C., and allyl magnesium bromide (170 mL of a 1M solution in diethyl ether, 170 mmol) was added dropwise. The resulting mixture was stirred for 1 h at RT, then cooled to −78° C. and a solution of (S)-citronellal (25 mL, 162 mmol) in diethyl ether (50 mL) was added dropwise over 1 h. The reaction was quenched by addition of methanol (30 mL) at −78° C., followed by simultaneous dropwise addition of 3N aqueous NaOH (122 mL) and hydrogen peroxide in water (146 mL of 30% wt solution) at 0° C., then stirred overnight at room temperature. Typical aqueous reductive workup and chromatography provided 27 g of product. Spectral data confirmed the structure of the product.

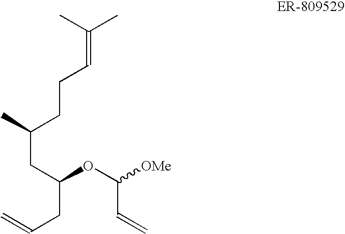

4-(1-Methoxy-allyloxy)-6,10-dimethyl-undeca-1,9-diene. A stainless reaction vessel was charged with a solution of the alcohol (26 g, 133 mmol) in acetonitrile (210 mL). The solution was degassed with nitrogen for 15-30 min, then Pd(OAc)₂ (1.5 g, 6.67 mmol), 1,3-bis(diphenyl)phosphinopropane (2.7 g, 6.67 mmol) and triethylamine (28 mL, 200 mmol) were introduced followed by freshly prepared 1-methoxy-1,2-propadiene (47 g, 665 mmol; prepared according to the procedure described by Weiberth and Hall, J. Org. Chem. 1985, 50, 5308). The reaction vessel was sealed and the mixture was stirred for 6 h at 80° C., additional 1-methoxy-1,2-propadiene was added as necessary to drive the reaction to completion. Concentration and chromatography produced 25 g of product. Spectral data confirmed the structure of the product.

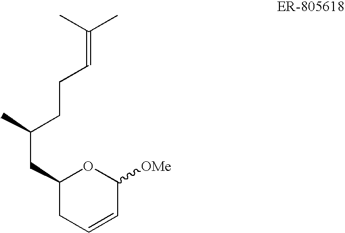

2-(2,6-Dimethyl-hept-5-enyl)-6-methoxy-3,6-dihydro-2H-pyran. A round-bottom flask charged with the starting material (39.9 g, 150 mmol) was purged with nitrogen twice before dichloromethane (350 mL) was introduced. Bis(tricyclohexylphosphine)benzylidene ruthenium (IV) dichloride (6.2 g, 7.5 mmol) was added and the resulting solution was stirred at −40° C. for ˜7 h, then cooled to RT and Pb(OAc)₄ (5 g, 11 mmol) was added. The mixture was stirred overnight at RT. Concentration and chromatography yielded 32 g of product. Spectral data confirmed the structure of the product.

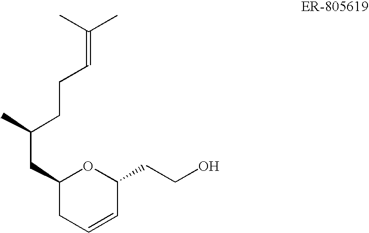

tert-Butyl-{2-[6-(2,6-dimethyl-hept-5-enyl)-5,6-dihydro-2H-pyran-2-yl]-ethoxy)-dimethyl-silane. LiClO₄ (165 g, 1.5 mol) was dissolved in diethyl ether (330 mL), cooled to 0° C., and a solution of the glycal (10.5 g, 0.044 mol) and the vinyl silyl ether (13.5 g, 0.088 mol) in EtOAc (40 mL) was added dropwise over 1 h, then allowed to warm to RT. Typical aqueous workup produced the crude aldehyde. The crude material was dissolved in THF (530 mL) and water (5.3 mL), cooled to 0° C., and solid NaBH₄ (3 g, 0.08 mol) was added. The reaction was allowed to stir at 0° C. until complete. Typical aqueous workup and chromatography provided 24.9 g of product. Spectral data confirmed the structure of the product.

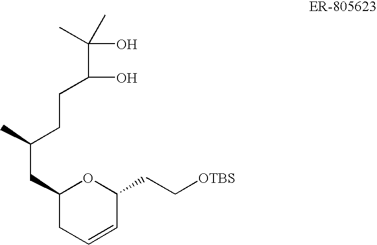

7-{6-[2-(tert-Butyl-dimethyl-silanyloxy)-ethyl]-3,6-dihydro-2H-pyran-2-yl}-2,6-dimethyl-heptane-2,3-diol. The alcohol (29.7 g, 0.12 mol) was dissolved in CH₂Cl₂ (500 mL) and sat. aq. NaHCO₃ (500 mL), cooled to 0° C., solid mCPBA (32.6 g, 0.188 mol) was added portionwise, and the reaction was stirred for 1 h. Typical aqueous workup provided 31.1 g of crude epoxide. The crude material (˜0.11 mol) was dissolved in THF (500 mL) and 0.1M aq. H₂SO₄ (500 mL) and stirred at RT for 1 h. Typical aqueous workup provided 35.5 a of crude triol. The crude material (˜0.11 mol) was dissolved in CH₂Cl₂ (600 mL), triethylamine (21 mL, 0.15 mol) and DMAP (1.4 g, 0.012 mol) were added, the solution cooled to 0° C., and TBSCl (21 g, 0.14 mol) was added. Reaction was allowed to warm to RT and stir for 4 h. Typical aqueous workup and chromatography provided 35 g of product. Spectral data confirmed the structure of the product. MS (API, ESP+) m/z 423 [M+Na]⁺.

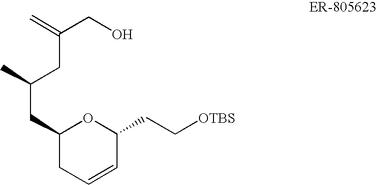

5-{6-[2-(tert-Butyl-dimethyl-silanyloxy)-ethyl]-3,6-dihydro-2H-pyran-2-yl}-4-methyl-2-methylene-pentan-1-ol. To a stirred solution of the diol (35 g, 87.5 mmol) in toluene (440 mL) at RT was added lead tetraacetate (78 g, 0.175 mol) portionwise, and the reaction was stirred for 2 h. Typical aqueous workup yielded 28.5 g crude aldehyde. The crude material was dissolved in CH₂Cl₂ (420 mL) and triethylamine (23 mL, 0.167 mol), Eschenmosher's salt (46.5 g, 0.25 mol) was added and the mixture was stirred overnight. Typical aqueous workup provided 32 g of crude enal. The crude material was dissolved in MeOH (400 mL), cooled to −78° C., and CeCl₃-7H₂O (31 g, 83 mmol) was added. A suspension of NaBH₄ (1.5 g, 41 mmol) in EtOH (50 mL) was added dropwise, and the reaction was stirred at −78° C. for 45 min. Typical aqueous workup and chromatography produced 16.7 g of product. Spectral data continued the structure of the product. MS (API, ESP+) m/z 377 [M+Na]⁺.

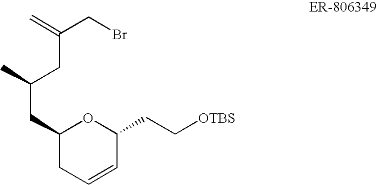

{2-[6-(4-Bromomethyl-2-methyl-pent-4-enyl)-5,6-dihydro-2H-pyran-2-yl]-ethoxy}-tert-butyl-dimethyl-silane. The alcohol (81 mg, 0.287 mmol) was dissolved in CH₂Cl₂ (2.5 mL), cooled to 0° C., and Ph₃P (79 mg, 0.300 mmol) and NBS (56 mg, 0.315 mmol) were added. The reaction mixture was stirred at 0° C. for 40 min. Typical buffered (pH 7) aqueous workup and chromatography produced 74 mg of product. Spectral data confirmed the structure of the product.

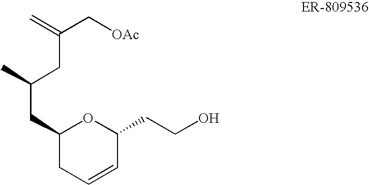

Acetic acid 5-[6-(2-hydroxy-ethyl)-3,6-dihydro-2H-pyran-2-yl]-4-methyl-2-methylene-pentyl ester. To a solution of the allylic alcohol (8.6 g, 24 mmol) in pyridine (35 mL) was added acetic anhydride (3.4 mL, 36 mmol) followed by DMAP (147 mg, 1 mmol), and the reaction was stirred for 90 min. Typical aqueous workup provided 9.3 g of crude acetate. The crude material was dissolved in acetonitrile (80 mL), treated with H₂SiF₆ (2.8 mL of a 20-30% wt aqueous solution), and stirred at RT for 30 min. Typical aqueous workup and chromatography produced 6.1 g of product. Spectral data continued the structure of the product. MS (API, ESP+) m/z 305 [M+Na]⁺.

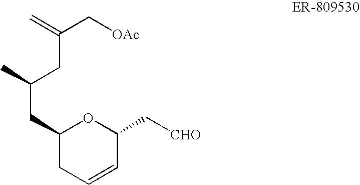

Acetic acid 4-methyl-2-methylene-5-[6-(2-oxo-ethyl)-3,6-dihydro-2H-pyran-2-yl]-pentyl ester. DMSO (1.3 mL, 18.6 mmol) was dissolved in CH₂Cl₂ (90 mL), cooled to −78° C., oxalyl chloride (9.3 mL of a 2M solution in CH₂Cl₂, 18.6 mmol) was added, and the mixture was allowed to stir at −78° C. for 1 h. A solution of the alcohol (4.37 g, 15.5 mmol) in CH₂Cl₂ (15 mL) was added dropwise over a 15 min period, the resulting mixture was stirred at −78° C. for 2 h, then triethylamine (6.5 mL, 46.5 mmol) was added. The mixture was allowed to warm gradually to RT for 1 h. Typical aqueous workup and chromatography produced 3.91 g of product. Spectral data confirmed the structure of the product. MS (API, ESP+) m/z 303 [M+Na]⁺.

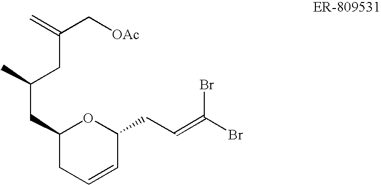

Acetic acid 5-[6-(3,3-dibromo-allyl)-3,6-dihydro-2H-pyran-2-yl]-4-methyl-2-methylene-pentyl ester. A stock solution was prepared as follows: PPh₃ (26.2 g, 0.10 mol) was dissolved in CH₂Cl₂ (100 mL), cooled to 0° C., CBr₄ (16.6 g, 0.05 mol) was added and the mixture was allowed to stir at 0° C. for 10 min and 30 min at RT. A fraction of the stock solution (60 mL, 0.03 mol) was cooled to −78° C., and a solution of the aldehyde (4.13 g, 0.015 mol) in CH₂Cl₂ (10 mL) was added dropwise over a period of 30 min. Additional amounts of the stock solution are added as necessary until completion of the reaction. Typical aqueous workup and chromatography produced 5.28 g of product. Spectral data confirmed the structure of the product. MS (API, ESP+) m/z 459 [M+Na]⁺.

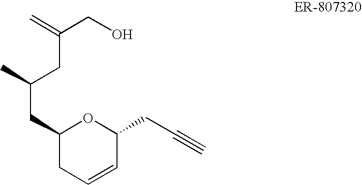

4-Methyl-2-methylene-5-(6-prop-2-ynyl-3,6-dihydro-2H-pyran-2-yl)-pentan-1-ol. The vinyl dibromide (7.3 g, 16.7 mmol) was dissolved in THF (80 mL), cooled to −78° C., and n-butyllithium (26 mL of a 2.5M solution in hexanes, 66.8 mmol) was added dropwise over 30 min. Typical aqueous workup and chromatography produced 3.8 g of product. Spectral data confirmed the structure of the product. MS (API, ESP+) m/z 257 [M+Na]⁺.

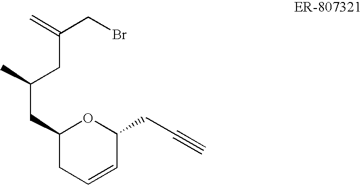

2-(4-Bromomethyl-2-methyl-pent-4-enyl)-6-prop-2-ynyl-3,6-dihydro-2H-pyran. The allylic alcohol (3.19 g, 13.7 mmol) was dissolved in CH₂Cl₂ (100 mL), cooled to 0° C., PPh₃ (4.6 g, 17.7 mmol) was added followed by N-bromosuccinimide (3.4 g, 19 mmol), and the reaction was stirred 1 h. Typical aqueous workup and chromatography produced 3.59 g of product. Spectral data confirmed the structure of the product.

### Example 7

### Preparation of ER-807320

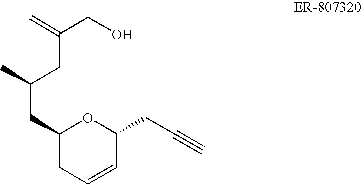

Exemplary Synthesis:

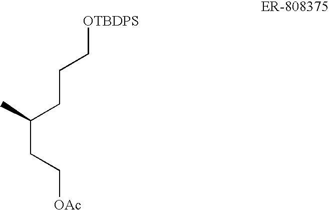

Acetic acid 6-(tert-butyl-diphenyl-silanyloxy)-3-methyl-hexyl ester. A three-necked flask was charged with (S)-citronellol (600 g, 3.85 mol), CH₂Cl₂ (4 L) and pyridine (1 L). Solid N,N-dimethylaminopyridine (36 g, 0.3 mol) was added to the mechanically stirred solution stirred followed by dropwise addition of acetic anhydride (545 mL, 5.78 mol) at 25° C. under nitrogen. Typical workup gave 800 g of product. For safety reasons, the ozonolysis of this material was performed in four batches of ˜200 g as follows: the crude acetate (284 g, 1.43 mol) was dissolved in CH₂Cl₂ (3 L) and MeOH (1 L) and cooled to −20° C. A stream of ozone was bubbled through the solution for 8 h at −20° C., then the solution was added dropwise to a solution of dimethylsulfide (1.25 L) in MeOH (2 L). Evaporation to dryness gave the crude aldehyde (245 g, 1.43 mol) that was dissolved in MeOH (3 L) and the resulting solution was cooled to 0° C. Solid NaBH₄ (37 g, 1 mol) was added portionwise and the reaction was allowed to stir mechanically for 4 h. Typical aqueous workup gave the alcohol. The crude alcohol (253 g, 1.43 mol) was charged in a three-necked flask and dissolved in DMF (2 L). Solid imidazole (292 g, 4.29 mol) and N,N-dimethylaminopyridine (17 g, 0.14 mol) were added followed by dropwise addition of tert-butyldiphenylchlorosilane (446 mL, 1.34 mol) at 25° C. under nitrogen. After completion of the addition, the reaction was stirred mechanically for several hours. Typical aqueous workup and purification by chromatography gave 580 g of the product. Spectral data confirmed the structure of the product.

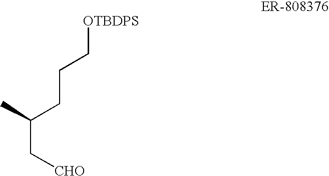

6-(tent-Butyl-diphenyl-silanyloxy)-3-methyl-hexanal. A three-necked flask was charged with the acetate (985 g, 2.39 mol) that was dissolved in MeOH (2.4 L) and H₂O (2.4 L). Solid K₂CO₃ (1.6 kg, 11.95 mol) was added and the resulting slurry was stirred mechanically at 60° C. overnight. Typical aqueous workup gave 880 g of the alcohol. This crude material was processed in batches in the Swern oxidation as follows: a three-necked flask was charged with DMSO (200 mL, 2.81 mol) and anhydrous CH₂Cl₂ (3 L). The solution was cooled to −78° C. and oxalyl chloride (245 mL, 2.81 mol) was added dropwise and the resulting solution was stirred mechanically for 1 h. Then, a solution of the alcohol (628 g, 1.69 mol) in CH₂Cl₂ (1 L) was added dropwise at −78° C. over a period of 1 h. Two hours later, Et₃N (700 mL, 5.07 mol) was added and the reaction was carried out for an additional 2 h at −78° C. Typical workup and chromatography gave 309 g of the aldehyde. Spectral data confirmed the structure of the product.

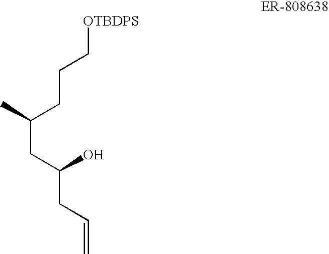

9-(tert-Butyl-diphenyl-silanyloxy)-6-methyl-non-1-en-4-ol. (−)-B-Methoxydiisopinocampheylborane (100 g, 316 mmol) was dissolved in diethyl ether (320 mL), cooled to 0° C., and allyl magnesium bromide (287 mL of a 1M solution in diethyl ether, 287 mmol) was added dropwise over an hour. The resulting mixture was stirred for an additional 1 h at RT. Then, diethyl ether was removed and the residue was taken into anhydrous pentane. The resulting suspension was filtered through celite under nitrogen and washed with anhydrous pentane. The clear solution was cooled to −100° C. under nitrogen and a solution of the aldehyde (70.7 g, 192 mmol) in diethyl ether (250 mL) was added dropwise over 2 h. The reaction was quenched by addition of methanol (50 mL) at −100° C., then the mixture was allowed to warm to 0° C. Aqueous 3 N NaOH (150 mL) and hydrogen peroxide in water (250 mL of 30% wt solution) were added drowise and the slurry was stirred overnight at room temperature. Typical aqueous reductive workup and chromatography provided 50 g of product. Spectral data confirmed the structure of the product.

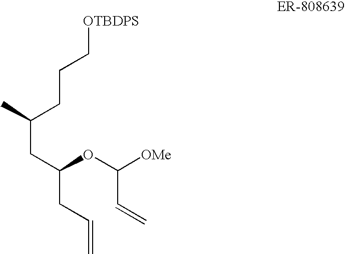

1-[1-Allyl-6-(tert-butyl-diphenyl-silanyloxy)-3-methyl-hexyloxy]-prop-2-en-1-ol. A stainless reaction vessel was charged with a solution of the alcohol (60 g, 146 mmol) in acetonitrile (300 mL). The solution was degassed with nitrogen for 15-30 min, then Pd(OAc)₂ (1.6 g, 7.3 mmol), 1,3-bis(diphenyl)phosphinopropane (3 g, 7.3 mmol) and triethylamine (30 mL, 219 mmol) were introduced followed by freshly prepared 1-methoxy-1,2-propadiene (71 g, 1 mmol; prepared according to the procedure described by Weiberth and Hall, J. Org. Chem. 1985, 50, 5308). The reaction vessel was sealed and the mixture was stirred for 6 h at 80° C., additional 1-methoxy-1,2-propadiene was added as necessary to drive the reaction to completion. Concentration and chromatography produced 39 g of product. Spectral data confirmed the structure of the product. MS (API, ESP+) m/z 504 [M+Na]⁺.

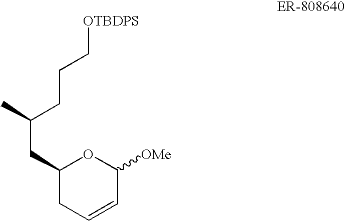

tert-Butyl-[5-(6-methoxy-3,6-dihydro-2H-pyran-2-yl)-4-methyl-pentyloxy]-diphenyl-silane. A round-bottom flask charged with the starting material (56 g, 116 mmol) was purged with nitrogen twice before dichloromethane (1.3 L) was introduced. Bis(tricyclohexylphosphine)benzylidene ruthenium (IV) dichloride (4.8 g, 5.8 mmol) was added and the resulting solution was stirred at 25° C. for several hours. Once the reaction was complete, Pb(OAc)₄ (2.7 g, 6 mmol) was added to the reaction, which was stirred for an additional 24 h. Concentration and chromatography yielded 48 g of product. Spectral data confirmed the structure of the product. MS (API, ESP+) m/z 476 [M+Na]⁺.

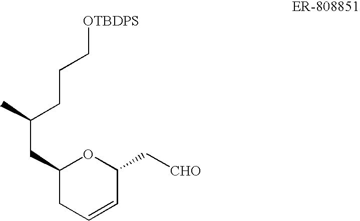

{6-[5-(tert-Butyl-diphenyl-silanyloxy)-2-methyl-pentyl]-5,6-dihydro-2H-pyran-2-yl}-acetaldehyde. The glycal (65.5 g, 0.144 mol) was dissolved in dry CH₂Cl₂ (500 mL) and vinyl silyl ether (49.8 g, 0.315 mol, prepared according to the procedure described by Srisiri et al, J. Org. Chem. 1994, 59, 5432). The mixture was cooled at 0° C. and Montmorillonite K-10 (32.8 g) was added. The reaction was allowed to stir for 1 h at 0° C. Filtration, concentration of the filtrate, and chromatography gave 32.3 g of product. Spectral data confirmed the structure of the product.

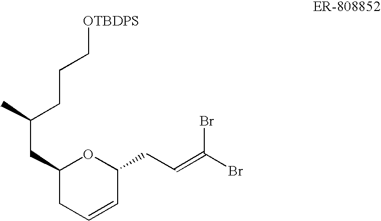

tert-Butyl-{5-[6-(3,3-dibromo-allyl)-3,6-dihydro-2H-pyran-2-yl]-4-methyl-pentyloxy}-diphenyl-silane. A stock solution was prepared as follows: PPh₃ (105 g, 0.4 mol) was dissolved in CH₂Cl₂ (400 mL), cooled to 0° C., CBr₄ (66.3 g, 0.2 mol) was added and the mixture was allowed to stir at 0° C. for 10 min and 30 min at RT. A fraction of the stock solution (275 mL. 0.138 mol) was cooled to −78° C., and a solution of the aldehyde (32.2 g, 68.9 mmol) in CH₂Cl₂ (175 mL) was added dropwise over a period of 30 min. Additional amounts of the stock solution were added as necessary until completion of the reaction. Typical aqueous workup and chromatography produced 26 g of product. Spectral data confirmed the structure of the product.

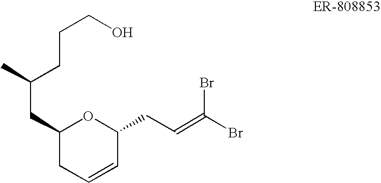

5-[6-(3,3-Dibromo-allyl)-3,6-dihydro-2H-pyran-2-yl]-4-methyl-pentan-1-ol. To a stirred solution of the silyl ether (26 g, 42 mmol) in THF (100 mL) at 0° C. was added a 1 M solution of TBAF in THF (42 mL, 42 mmol) and the mixture was allowed to warm gradually to room temperature. Additional amounts of the TBAF solution were added as necessary until completion of the reaction. Typical aqueous workup and chromatography produced 9.8 g of product. Spectral data confirmed the structure of the product.

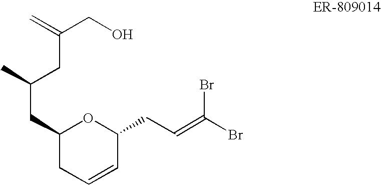

5-[6-(3,3-Dibromo-allyl)-3,6-dihydro-2H-pyran-2-yl]-4-methyl-2-methylene-pentan-1-ol. A three-necked flask was charged with DMSO (10.9 mL, 0.15 mol) and anhydrous CH₂Cl₂ (100 mL). The solution was cooled to −78° C. and a 2 M solution of oxalyl chloride in CH₂Cl₂ (38.5 mL, 76.9 mmol) was added dropwise and the resulting solution was stirred mechanically for 15 min at −78° C. Then, a solution of the alcohol (9.8 g, 25.6 mmol) in CH₂Cl₂ (50 mL) was added dropwise at −78° C. over a period of 1 h. Two hours later, Et₃N (60 mL, 128 mmol) was added and the reaction was carried out for an additional 2 h at 25° C. Then, a first portion of solid Eschenmoser's salt (23.7 g, 128 mmol) followed by another portion (10 g, 54 mmol) were added directly to the reaction mixture, which was stirred overnight at 25° C. under nitrogen. Typical workup gave 19 g of the crude intermediate that was dissolved a mixture of diethyl ether (330 mL) and CHCl₃ (170 mL) and treated with iodomethane (30 mL, 485 mmol). The resulting suspension was stirred overnight at 25° C. under nitrogen. The reaction mixture was concentrated to dryness and the resulting crude material was dissolved in CH₂Cl₂ (200 mL) and saturated aqueous K₂CO₃ solution (200 mL). The resulting suspension was stirred overnight at 25° C. Usual aqueous workup generated 15 g of a crude oil that was dissolved in MeOH (125 mL) and cooled to −78° C. Solid CeCl₃ 7 H₂O (11.4 g, 30.7 mmol) followed by solid NaBH₄ (1.1 g, 30.7 mmol) were added and the resulting mixture was stirred at −78° C. for 15 min. Typical aqueous workup and chromatography produced 4.2 g of product. Spectral data confirmed the structure of the product.

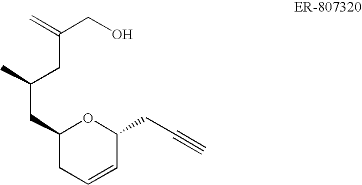

4-Methyl-2-methylene-5-(6-prop-2-ynyl-3,6-dihydro-2H-pyran-2-yl)-pentan-1-ol. The vinyl dibromide (4.2 g, 10.7 mmol) was dissolved in THF (50 mL), cooled to −78° C., and n-butyllithium (19 mL of a 2.5M solution in hexanes, 47.5 mmol) was added dropwise over 30 min. Typical aqueous workup and chromatography produced 2.17 g of product. Spectral data confirmed the structure of the product. MS (API, ESP+) m/z 257 [M+Na]⁺.

### Example 8

### Preparation of ER-808426 and Derivatives/Analogues Thereof

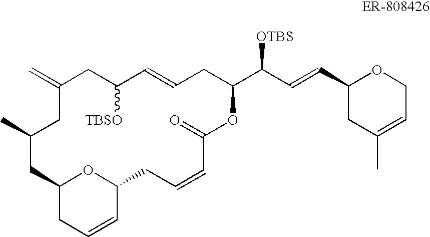

Exemplary synthesis:

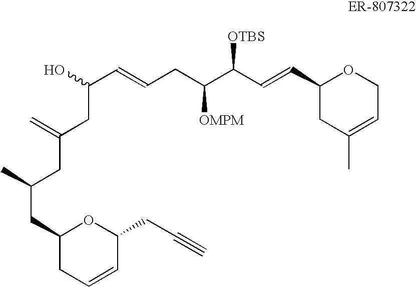

11-(tert-Butyl-dimethyl-silanyloxy)-10-(4-methoxy-benzyloxy)-2-methyl-13-(4-methyl-3,6-dihydro-2H-pyran-2-yl)-4-methylene-1-(6-prop-2-ynyl-3,6-dihydro-2H-pyran-2-yl)-trideca-7,12-dien-6-ol. The enal (6.85 g, 14.1 mmol) and bromide (4.62 g, 15.5 mmol) were dissolved in a 3/1 mixture of THF/H₂O (240 mL), 0.4M aq. HCl (10 mL) was added, followed by In powder (6.50 g, 56.6 mmol). The mixture was stirred at RT overnight, then filtered and subjected to typical aqueous workup and chromatography to produce 9.01 g of product as a ˜1:1 mixture of diastereomers. Spectral data confirmed the structure of the product. MS (API, ESP+) m/z 727 [M+Na]⁺.

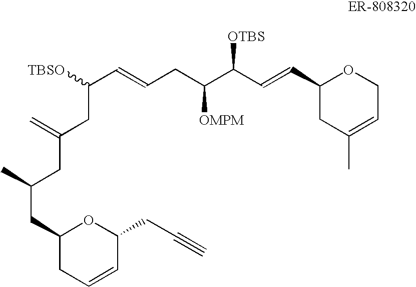

6,11-Bis-(tert-butyl-dimethyl-silanyloxy)-10-(4-methoxy-benzyloxy)-2-methyl-13-(4-methyl-3,6-dihydro-2H-pyran-2-yl)-4-methylene-1-(6-prop-2-ynyl-3,6-dihydro-2H-pyran-2-yl)-trideca-7,12-diene. The alcohol (9.01 g, 12.78 mmol) was dissolved in CH₂Cl₂ (200 mL), pyridine (2.6 mL, 32.15 mmol) was added followed by TBSOTf (4.4 mL, 19.16 mmol) and DMAP (118 mg, 0.95 mmol). After ˜30 min, typical aqueous workup and chromatography produced 9.67 g of product. Spectral data confirmed the structure of the product. MS (API, ESP+) m/z 842 [M+Na]⁺.

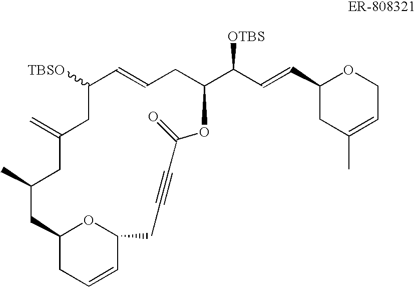

11-(tert-Butyl-dimethyl-silanyloxy)-7-[1-(tert-butyl-dimethyl-silanyloxy)-3-(4-methyl-3,6-dihydro-2H-pyran-2-yl)-allyl]-15-methyl-13-methylene-6,21-dioxa-bicyclo[15.3.1]henicosa-9,19-dien-3-yn-5-one. The alkyne (9.30 g, 11.35 mmol) was dissolved in THF (400 mL), cooled to −78° C., and n-butyllithium (14.0 mL of a 2.5M solution in hexanes, 35.0 mmol) was added slowly. After 1 min, dry ice (rinsed with anhyrous THF then crushed) was added, the reaction was stirred for 10 min, and then allowed to warm to RT. Typical aqueous workup yielded the crude alkynoic acid product. The crude material was dissolved in CH₂Cl₂ (200 mL) and phosphate buffer pH 7 (200 mL), DDQ (10.41 g, 45.86 mmol) was added, and the reaction was stirred at RT for ˜1 h. Typical aqueous workup provided the crude alcohol. The crude material was processed in three portions as follows: The crude material was dissolved in THF (25 mL), i-Pr₂NEt (2.3 mL, 13.2 mmol) was added followed by trichlorobenzoyl chloride (0.89 mL, 1.39 g, 5.70 mmol). The reaction was stirred for 1.5 h then diluted with toluene (15 mL) and added via syringe pump (9 mL/h rate) to a solution of DMAP (4.72 g, 38.63 mmol) in toluene (750 mL). Typical aqueous workup and chromatography produced 4.18 g of product. Spectral data confirmed the structure of the product. MS (API, ESP+) m/z 747 [M+Na]⁺.

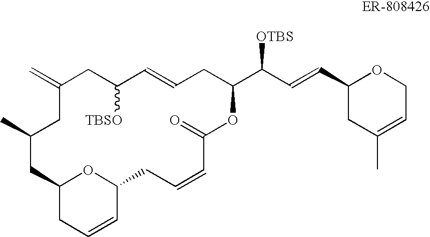

11-(tert-Butyl-dimethyl-silanyloxy)-7-[1-(tert-butyl-dimethyl-silanyloxy)-3-(4-methyl-3,6-dihydro-2H-pyran-2-yl)-allyl]-15-methyl-13-methylene-6,21-dioxa-bicyclo[15.3.1.]henicosa-3,9,19-trien-5-one. The macrocycle (2.91 g, 4.01 mmol) was dissolved in CH₂Cl₂ (150 mL) and hexanes (450 mL), quinoline (1.9 mL, 2.1 g, 16.1 mmol) was added followed by Lindlar catalyst (2.89 g). The mixture was subjected to three vacuum/purge cycles and then stirred under H₂ (balloon) for 1.5 h. Filtration, concentration, and chromatography produced 2.38 g of product. Spectral data confirmed the structure of the product. MS (API, ESP+) m/z 749 [M+Na]⁺.

For purification of diastereomeric mixture and use of ER-808426 in the synthesis of Laulimalide analogues, see Example 13.

### Example 9

### Alternative Synthesis of ER-809587 via Horner-Wadsworth-Emmons Methodology

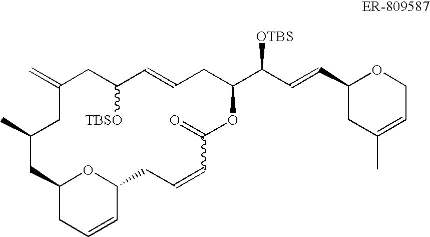

Exemplary Synthesis:

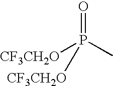

Methyl-phosphonic acid bis-(2,2,2-trifluoro-ethyl)ester. A modification of the procedure by Patois et al (Synth. Commun. 1991, 21, 2391) was used. Triethylamine (58 mL, 42 g, 416 mmol) and 2,2,2-trifluoroethanol (27.6 mL, 37.8 g, 378 mmol) were dissolved in THF (300 mL), cooled to 0° C., and a solution of the methylphosphonic dichloride (25 g, 188 mmol) in THF (50 mL) was added dropwise. Typical aqueous workup and distillation provided 27.6 g of product. Spectral data confirmed the structure of the product.

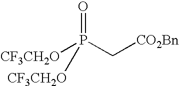

[Bis-(2,2,2-trifluoro-ethoxy)-phosphoryl]-acetic acid benzyl ester. LiHMDS (21 mL of a 1M solution in THF, 21 mmol) was cooled to −78° C., a solution of the phosphonate (2.63 g, 10.11 mmol) and benzyl chloroformate (1.55 g, 1.85 mL, 10.86 mmol) in THF (35 mL) was added dropwise, and the reaction was allowed to slowly warm to 0° C. Typical aqueous workup, Kugelrohr distillation, and chromatography provided 0.88 g of product. Spectral data confirmed the structure of the product. MS (API, ESP+) m/z 417 [M+Na]⁺.

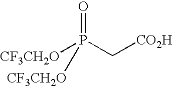

[Bis-(2,2,2-trifluoro-ethoxy)-phosphoryl]-acetic acid. The benzyl ester (0.52 g, 1.31 mmol) and Pd/C (47 mg of a 10% wt palladium on carbon) were suspended in EtOAc (5 mL), the mixture was subjected to three vacuum/purge cycles and then stirred under H₂ (balloon) for 2.5 h. Filtration and concentration produced 0.35 g of product. Spectral data confirmed the structure of the product.

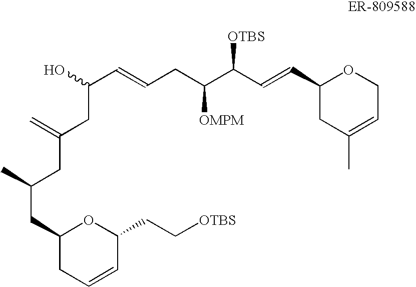

11-(tert-Butyl-dimethyl-silanyloxy)-1-{6-[2-(tert-butyl-dimethyl-silanyloxy)-ethyl]-3,6-dihydro-2H-pyran-2-yl}-10-(4-methoxy-benzyloxy)-2-methyl-13-(4-methyl-3,6-dihydro-2H-pyran-2-yl)-4-methylene-trideca-7,12-dien-6-ol. The enal (241 mg, 0.496 mmol) and bromide (231 mg, 0.554 mmol) were dissolved in a 1/2 mixture of THF/H₂O (6 mL), and In powder (6.50 g, 56.6 mmol) was added. Filtration, typical aqueous workup and chromatography produced 318 mg of product. Spectral data confirmed the structure of the product as a ˜1:1 mixture of diastereomers. MS (API, ESP+) m/z 847 [M+Na]⁺.

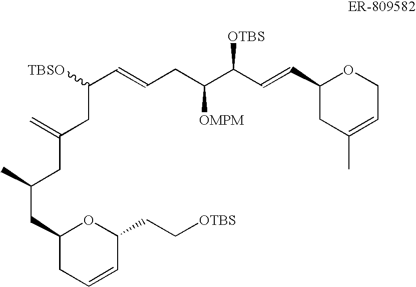

3,8-Bis-(tert-butyl-dimethyl-silanyloxy)-13-}6-[2-(tert-butyl-dimethyl-silanyloxy)-ethyl]-3,6-dihydro-2H-pyran-2-yl}-4-(4-methoxy-benzyloxy)-12-methyl-1-(4-methyl-3,6-dihydro-2H-pyran-2-yl)-10-methylene-trideca-1,6-diene. The alcohol (234 mg, 0.28 mmol) and DMAP (9.9 mg, 0.08 mmol) were dissolved in pyridine (2.5 mL) and TBSOTf (0.18 mL, 0.21 g, 0.79 mmol) was added. Typical aqueous workup and chromatography produced 192 mg of product. Spectral data confirmed the structure of the product.

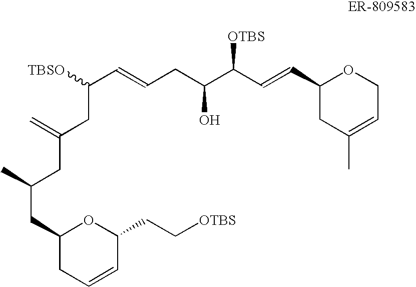

3,8-Bis-(tert-butyl-dimethyl-silanyloxy)-13-{6-[2-(tert-butyl-dimethyl-silanyloxy)-ethyl]-3,6-dihydro-2H-pyran-2-yl}-12-methyl-1-(4-methyl-3,6-dihydro-2H-pyran-2-yl)-10-methylene-trideca-1,6-dien-4-ol. The starting material (192 mg, 0.20 mmol) was dissolved in CH₂Cl₂ (4 mL) and phosphate buffer pH 7 (4 mL) and DDQ (78 mg, 0.34 mmol) was added. Typical aqueous workup and chromatography provided 126 mg of product. Spectral data confirmed the structure of the product. MS (API, ESP+) m/z 841 [M+Na]⁺.

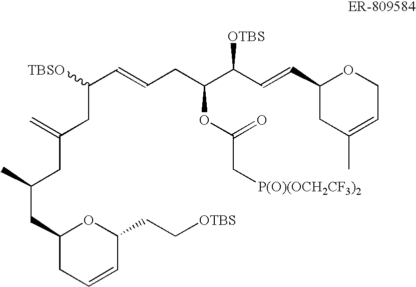

[3,8-Bis-(tert-butyl-dimethyl-silanyloxy)-4-[bis-(2,2,2-trifluoro-ethyl)-phosphoryl acetoxy]-13-{6-[2-(tert-butyl-dimethyl-silanyloxy)-ethyl]-3,6-dihydro-2H-pyran-2-yl}-12-methyl-1-(4-methyl-3,6-dihydro-2H-pyran-2-yl)-10-methylene-trideca-1,6-diene. The alcohol (62.5 mg, 0.076 mmol) was dissolved in CH₂Cl₂ (3 mL), the acid (0.25 g, 0.81 mmol) was added, followed by HOBt-H₂O (11.5 mg, 0.085 mmol) and EDCI-MeI (0.22 g, 0.74 mmol). Direct chromatography produced 72 mg of product. Spectral data confirmed the structure of the product.

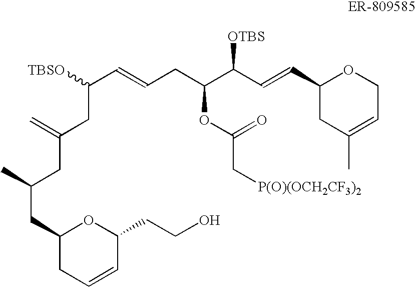

3,8-Bis-(tert-butyl-dimethyl-silanyloxy)-4-[bis-(2,2,2-trifluoro-ethyl)-phosphoryl acetoxy]-13-{6-[ethyl-2-ol]-3,6-dihydro-2H-pyran-2-yl}-12-methyl-1-(4-methyl-3,6-dihydro-2H-pyran-2-yl)-10-methylene-trideca-1,6-diene. The phosphonate (58.9 mg, 0.053 mmol) was dissolved in HOAC (2 mL), THF (2 mL), and H₂O (2 mL). Typical aqueous workup and chromatography provided 12.1 mg of product and 39.4 mg of recovered starting material. Spectral data confirmed the structure of the product.

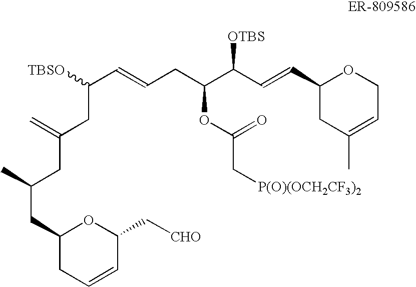

3,8-Bis-(tert-butyl-dimethyl-silanyloxy)-4-[bis-(2,2,2-trifluoro-ethyl)-phosphoryl acetoxy]-13-{6-[2-oxo-ethyl]-3,6-dihydro-2H-pyran-2-yl}-12-methyl-1-(4-methyl-3,6-dihydro-2H-pyran-2-yl)-10-methylene-trideca-1,6-diene. The alcohol (24 mg, 0.025 mmol) was dissolved in CH₂Cl₂ (5 mL), solid NaHCO₃ (0.12 g, 1.48 mmol) and t-BuOH (105 uL) were added followed by Dess-Martin periodane (0.11 g, 0.26 mmol). Direct chromatography produced 28 mg of crude product. Spectral data confirmed the structure of the product. MS (API, ESP+) m/z 1011 [M+Na]⁺.

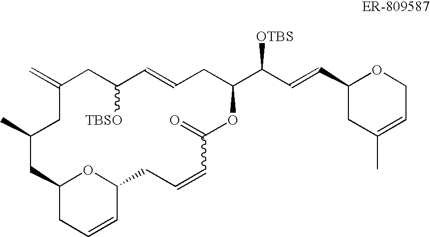

11-(tert-Butyl-dimethyl-silanyloxy)-7-[1-(tert-butyl-dimethyl-silanyloxy)-3-(4-methyl-3,6-dihydro-2H-pyran-2-yl)-allyl]-15-methyl-13-methylene-6,21-dioxa-bicyclo[15.3.1]henicosa-3,9,19-trien-5-one. K₂CO₃ (26 mg, 0.19 mmol) and 18-crown-6 (101 mg, 0.38 mmol) were dissolved in toluene (30 mL), cooled to −40° C., and a solution of the phosphonate (24 mg, 0.025 mmol) in toluene (10 mL) was added. The reaction was stirred at −40° C. for ˜2 h, then slowly warmed to RT. Typical aqueous workup and chromatography yielded 11.2 mg of product. Spectral data confirmed the structure of the products as a ˜2.3:1 mixture of E:Z. MS (API, ESP+) m/z 749 [M+Na]⁺.

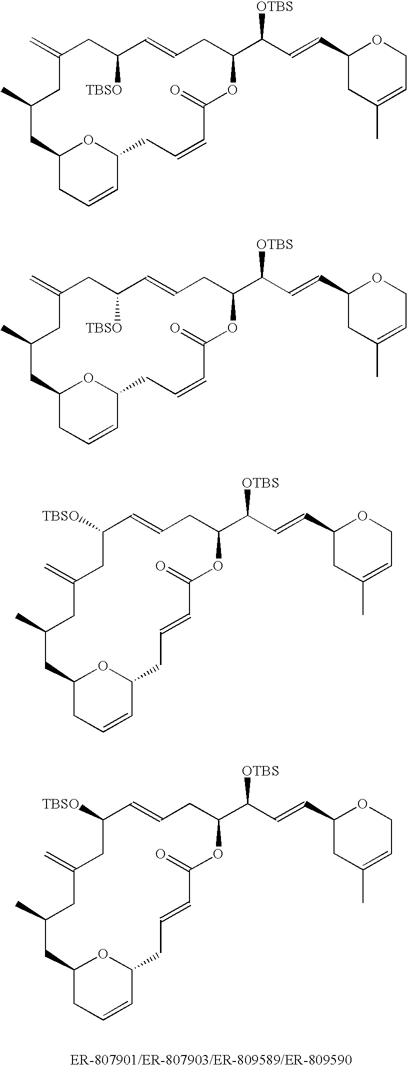

HPLC purification of the C.15 diastereomers. The mixture of diastereomers was separated by semi-prep HPLC using Chiralpak AD semi-prep column and a 1% IPA/hexanes mobile phase. Spectral data confirmed the structure of all products and MS (API, ESP+) for each compound m/z 749 [M+Na]⁺.

### Example 10

### Laulimalide Analogues Derived from ER-809587

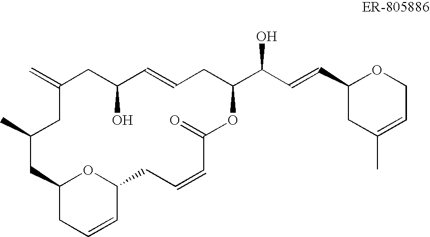

Des-epoxy-laulimalide. The bis-TBS compound (1.8 mg, 0.0025 mmol) was dissolved in CH₃CN (0.5 mL), and H₂SiF₆ (1 drop of a 20-25% wt aqueous solution) was added. Chromatography produced 0.8 mg of product. Spectral data confirmed the structure of the product. MS (API, ESP+) m/z 521 [M+Na]⁺.

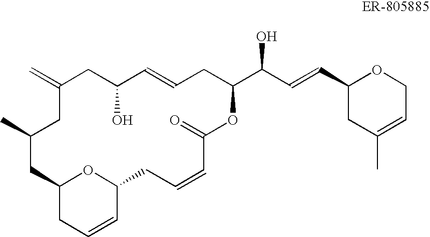

C.15-epi des-epoxy-laulimalide. The bis-TBS compound (2.5 mg, 0.0034 mmol) was dissolved in CH₃CN (0.5 mL), and H₂SiF₆ (1 drop of a 20-25% wt aqueous solution) was added. Chromatography produced 1.2 mg of product. Spectral data confirmed the structure of the product. MS (API, ESP+) m/z 521 [M+Na]⁺.

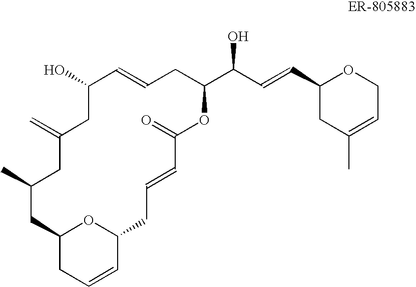

C.2-C.3-(E) des-epoxy-laulimalide. The bis-TBS compound (2.8 mg, 0.0039 mmol) was dissolved in CH₃CN (0.5 mL), and H₂SiF₆ (1 drop of a 20-25% wt aqueous solution) was added. Chromatography produced 2.0 mg of product. Spectral data confirmed the structure of the product. MS (API, ESP+) m/z 521 [M+Na]⁺.

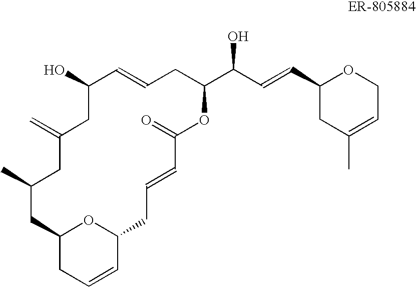

C.2-C.3-(E) C.15-epi des-epoxy-lautimalide. The bis-TBS compound (3.5 mg, 0.0048 mmol) was dissolved in CH₃CN (0.5 mL), and H₂SiF₆ (1 drop of a 20-25% wt aqueous solution) was added. Chromatography produced 2.2 mg of product. Spectral data confirmed the structure of the product. MS (API, ESP+) m/z 521[M+Na]⁺.

### Example 11

### Preparation of ER-807316 and Analogues Thereof

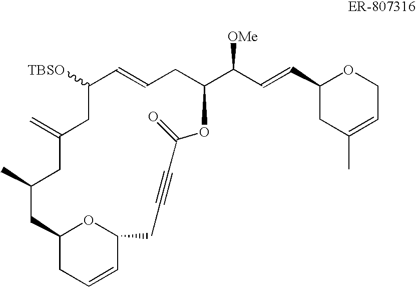

Exemplary Synthesis:

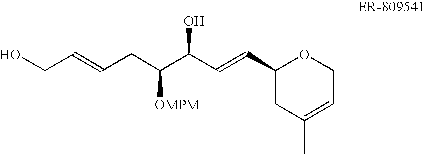

5-(4-Methoxy-benzyloxy)-8-(4-methyl-3,6-dihydro-2H-pyran-2-yl)-octa-2,7-diene-1,6-diol. The ester (0.18 g, 0.44 mmol) was dissolved in CH₂Cl₂ (4 mL), cooled to −78° C., and DIBAL (1.5 mL of a 1M solution in CH₂Cl₂, 1.5 mmol) was added dropwise. Typical aqueous workup and chromatography provided 80 mg of product. Spectral data confirmed the structure of the product. MS (API, ESP+) m/z 397 [M+Na]⁺.

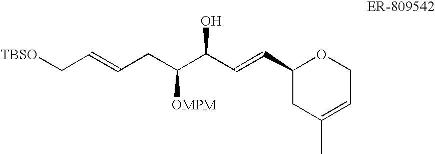

8-(tert-Butyl-dimethyl-silanyloxy)-4-(4-methoxy-benzyloxy)-1-(4-methyl-3,6-dihydro-2H-pyran-2-yl)-octa-1,6-dien-3-ol. The diol (80 mg, 0.21 mmol) was dissolved in CH₂Cl₂ (2 mL), cooled to 0° C., and 2,6-lutidine (50 μL, 0.43 mmol) and TBSOTf (58 μL, 0.26 mmol) were added. Additional TBSOTf was added as necessary to complete the reaction. Typical aqueous workup and chromatography provided 70 mg of product. Spectral data confirmed the structure of the product.

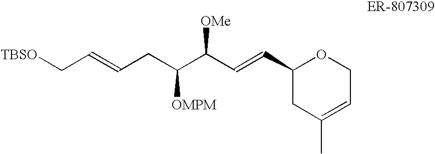

tert-Butyl-[6-methoxy-5-(4-methoxy-benzyloxy)-8-(4-methyl-3,6-dihydro-2H-pyran-2-yl)-octa-2,7-dienyloxy]-dimethyl-silane. The alcohol (60 mg, 0.12 mmol) was dissolved in THF (1.5 mL), and NaH (49 mg of a 60% dispersion in mineral oil, 1.23 mmol) and MeI (46 μL, 0.10 g, 0.74 mmol) were added. Typical aqueous workup and chromatography provided the 61 mg of product. Spectral data confirmed the structure of the product.

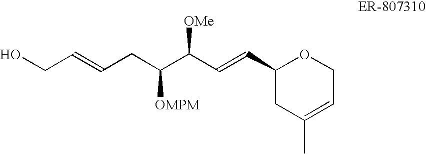

6-Methoxy-5-(4-methoxy-benzyloxy)-8-(4-methyl-3,6-dihydro-2H-pyran-2-yl)-octa-2,7-dien-1-ol. The methyl ether (61 mg, 0.12 mmol) and TBAF (0.15 mL of a 1M solution in THF, 0.15 mmol) were dissolved in THF (1 mL). Typical aqueous workup and chromatography provided the 44 mg of product. Spectral data confirmed the structure of the product. MS (API, ESP+) m/z 411 [M+Na]⁺.

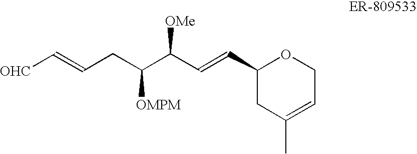

6-Methoxy-5-(4-methoxy-benzyloxy)-8-(4-methyl-3,6-dihydro-2H-pyran-2-yl)-octa-2,7-dienal. The allylic alcohol (45 mg, 0.12 mmol) was dissolved in CH₂Cl₂ (1 mL), and 4 Å molecular sieves (64 mg), tetrapropylammonium perruthenate (3.3 mg, 0.009 mmol) and N-methylmorpholine N-oxide (20 mg, 0.17 mmol) were added. Filtration through Silica gel and concentration provided 36 mg of product. Spectral data confirmed the structure of the product.

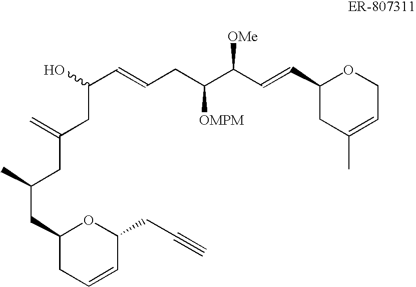

11-Methoxy-10-(4-methoxy-benzyloxy)-2-methyl-13-(4-methyl-3,6-dihydro-2H-pyran-2-yl)-4-methylene-1-(6-prop-2-ynyl-3,6-dihydro-2H-pyran-2-yl)-trideca-7,12-dien-6-ol. The enal (36 mg, 0.093 mmol) and bromide (33 mg, 0.11 mmol) were dissolved in a 3/1 mixture of THF/H₂O (2 mL), 0.4M aq. HCl (64 μL) was added, followed by In powder (43 g, 0.37 mmol). The mixture was stirred at RT overnight, then filtered and subjected to typical aqueous workup and chromatography to produce 45 mg of product. Spectral data confirmed the structure of the product as a ˜1:1 mixture of diastereomers.

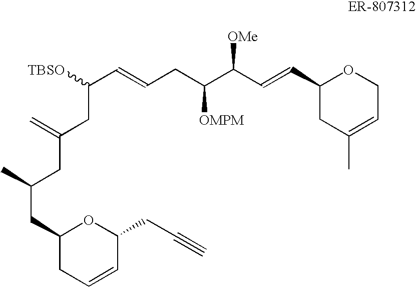

tert-Butyl-{6-methoxy-5-(4-methoxy-benzyloxy)-8-(4-methyl-3,6-dihydro-2H-pyran-2-yl)-1-[4-methyl-2-methylene-5-(6-prop-2-ynyl-3,6-dihydro-2H-pyran-2-yl)-pentyl]-octa-2,7-dienyloxy}-dimethyl-silane. The alcohol (45 mg, 0.074 mmol) and DMAP (several crystals) were dissolved in pyridine (1 mL) and TBSOTf (4 drops) was added. Typical aqueous workup and chromatography produced 43 mg of product. Spectral data confirmed the structure of the product.

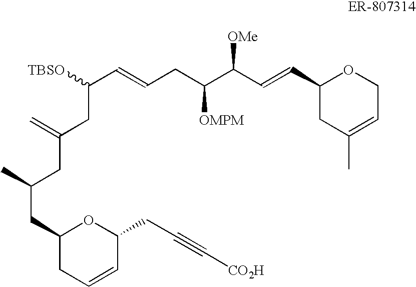

4-{6-[6-(tert-Butyl-dimethyl-silanyloxy)-11-methoxy-10-(4-methoxy-benzyloxy)-2-methyl-13-(4-methyl-3,6-dihydro-2H-pyran-2-yl)-4-methylene-trideca-7,12-dienyl]-5,6-dihydro-2H-pyran-2-yl}-but-2-ynoic acid. The alkyne (43 mg, 0.060 mmol) was dissolved in THF (0.4 mL), cooled to −78° C., and n-BuLi (0.20 mL of a 1.6M solution in hexanes, 0.32 mmol) was added. After ˜5 min, a stream of CO₂ gas was bubbled through the reaction mixture. Typical aqueous workup and chromatography provided 26 mg of product. Spectral data confirmed the structure of the product.

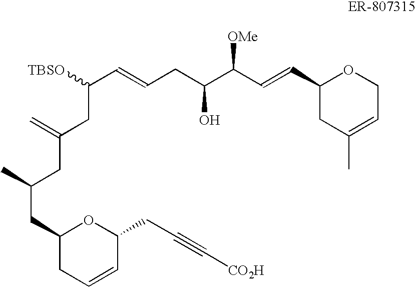

4-{6-[6-(tert-Butyl-dimethyl-silanyloxy)-10-hydroxy-11-methoxy-2-methyl-13-(4-methyl-3,6-dihydro-2H-pyran-2-yl)-4-methylene-trideca-7,12-dienyl]-5,6-dihydro-2H-pyran-2-yl}-but-2-ynoic acid. The acid (26 mg, 0.034 mmol) and DDQ (31 mg, 0.14 mmol) were dissolved in a 2/1 mixture of CH₂Cl₂/H₂O (0.6 mL). Typical aqueous workup and chromatography provided 16 mg of product. Spectral data confirmed the structure of the product. MS (API, ESP−) m/z 641 [M−H]⁻.

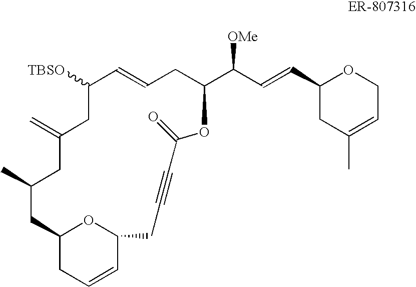

11-(tert-Butyl-dimethyl-silanyloxy)-7-[1-methoxy-3-(4-methyl-3,6-dihydro-2H-pyran-2-yl)-allyl]-15-methyl-13-methylene-6,21-dioxa-bicyclo[15.3.1]henicosa-9,19-dien-3-yn-5-one. The acid (16 mg, 0.025 mmol) was dissolved in THF (0.6 mL), i-Pr₂NEt (8.7 μL, 6.5 mg, 0.050 mmol) was added followed by trichlorobenzoyl chloride (6.2 μL, 9.7 mg, 0.040 mmol). The reaction was stirred for 2 h then diluted with toluene (9 mL) and added via syringe pump (˜6 mL/h rate) to a solution of DMAP (30 mg, 0.25 mmol) in toluene (9 mL). Typical aqueous workup and chromatography produced 9.8 mg of product. Spectral data confirmed the structure of the product. MS (API, ESP+) m/z 647 [M+Na]⁺.

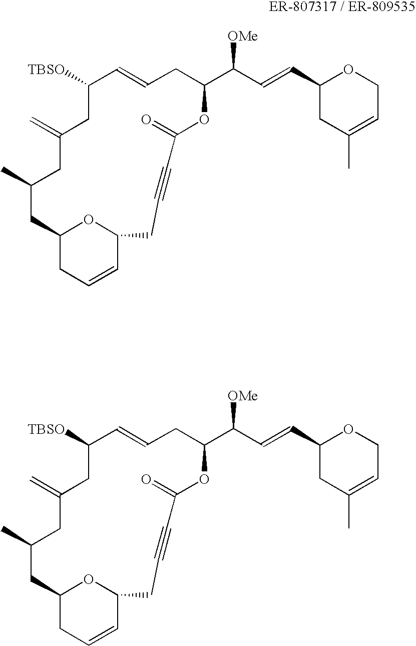

HPLC purification of the C.15 diastereomers. The mixture of diastereomers was separated by semi-prep HPLC using Chiralpak AD semi-prep column and a 1% IPA/hexanes mobile phase.

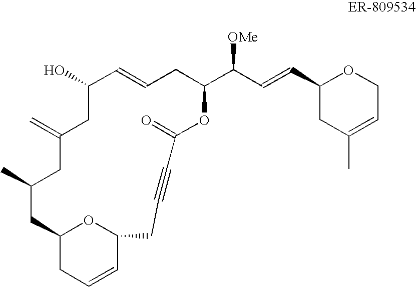

11-Hydroxy-7-[1-methoxy-3-(4-methyl-3,6-dihydro-2H-pyran-2-yl)-allyl]-15-methyl-13-methylene-6,21-dioxa-bicyclo[15.3.1]henicosa-9,19-dien-3-yn-5-one. The TBS ether (3.8 mg, 0.0061 mmol) was dissolved in CH₃CN (0.5 mL) and H₂SiF₆ (1 drop of a 20-25% wt aqueous solution) was added. Typical aqueous workup and chromatography produced 3.1 mg of product. Spectral data confirmed the structure of the product.

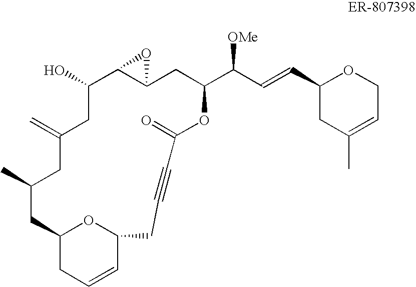

7-Hydroxy-12-[1-methoxy-3-(4-methyl-3,6-dihydro-2H-pyran-2-yl)-allyl]-3-methyl-5-methylene-9,13,22-trioxa-tricyclo[16.3.1.08,10]docos-19-en-15-yn-14-one. A stock solution was made as follows: (+)-DIPT (106 μL, 118 mg, 0.50 mmol) was dissolved in CH₂Cl₂ (1 mL), cooled to −40° C., Ti(OiPr)₄ (120 μL, 115 mg, 0.40 mmol) was added, the mixture stirred 5 min, then t-BuOOH (110 μL of a ˜5.5M solution in nonane, 0.61 mmol) was added and the solution stirred for an additional 15 min. The diol (3.4 mg, 0.0067 mmol) was dissolved in CH₂Cl₂ (0.4 mL), cooled to −20° C., and portions of the stock solution were added until the reaction was complete. Typical aqueous workup and chromatography produced 1.4 mg of product. Spectral data confirmed the structure of the product. MS (API, ESP+) m/z 549 [M+Na]⁺.

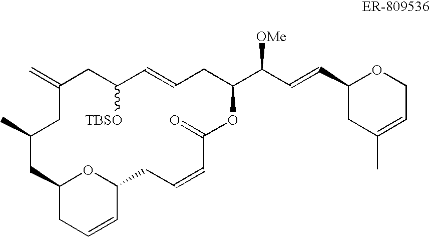

11-(tert-Butyl-dimethyl-silanyloxy)-7-[1-methoxy-3-(4-methyl-3,6-dihydro-2H-pyran-2-yl)-allyl]-15-methyl-13-methylene-6,21-dioxa-bicyclo[15.3.1]henicosa-3,9,19-trien-5-one. The alkynyl macrocycle (9.0 mg, 0.014 mmol) was dissolved in a 3/1 mixture of hexanes/CH₂Cl₂ (2 mL), quinoline (6.8 μL, 7.4 mg, 0.058 mmol) was added followed by Lindlar catalyst (10 mg). The mixture was subjected to three vacuum/purge cycles and then stirred under (balloon). Filtration, concentration, and chromatography produced 9 mg of product. Spectral data confirmed the structure of the product. MS (API, ESP+) m/z 649 [M+Na]⁺. An over-reduced side-product was also observed and confirmed by spectral data. MS (API, ESP+) m/z 651 [M+Na]⁺.

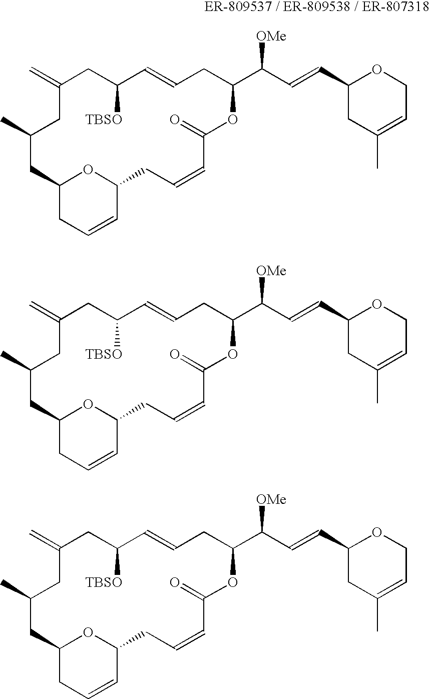

HPLC purification of the C.15 diastereomers and the over-reduced product. The mixture of isomers was separated by semi-prep HPLC using. Chiralpak AD semi-prep column and a 1% IPA/hexanes mobile phase.

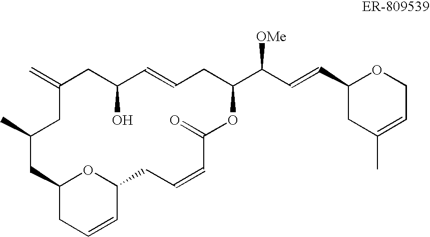

11-Hydroxy-7-[1-methoxy-3-(4-methyl-3,6-dihydro-2H-pyran-2-yl)-allyl]-15-methyl-13-methylene-6,21-dioxa-bicyclo[15.3.1]henicosa-3,9,19-trien-5-one. The TBS ether (4.0 mg, 0.0063 mmol) was dissolved in CH₃CN (0.5 mL) and H₂SiF₆ (1 drop of a 20-25% wt aqueous solution) was added. Typical aqueous workup and chromatography produced 3.0 mg of product. Spectral data confirmed the structure of the product.

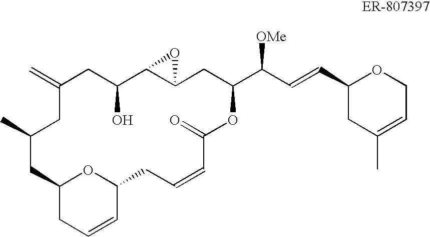

7-Hydroxy-12-[1-methoxy-3-(4-methyl-3,6-dihydro-2H-pyran-2-yl)-allyl]-3-methyl-5-methylene-9,13,22-trioxa-tricyclo[16.3.1.08,10]docosa-15,19-dien-14-one. A stock solution was made as follows: (+)-DIPT (106 μL, 118 mg, 0.50 mmol) was dissolved in CH₂Cl₂ (1 mL), cooled to −40° C., Ti(OiPr)₄ (120 μL, 115 mg, 0.40 mmol) was added, the mixture stirred 5 min, then t-BuOOH (110 μL of a ˜5.5M solution in nonane, 0.61 mmol) was added and the solution stirred for an additional 15 min. The diol (3.0 mg, 0.0058 mmol) was dissolved in CH₂Cl₂ (0.4 mL), cooled to −20° C., and portions of the stock solution were added until the reaction was complete. Typical aqueous workup and chromatography produced 1.8 mg of product. Spectral data confirmed the structure of the product. MS (API, ESP+) m/z 551 [M+Na]⁺.

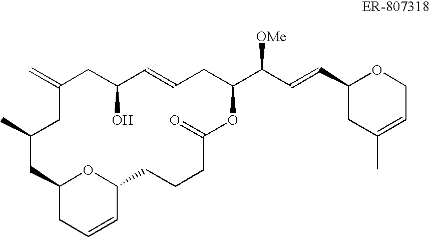

11-Hydroxy-7-[1-methoxy-3-(4-methyl-3,6-dihydro-2H-pyran-2-yl)-allyl]-15-methyl-13-methylene-6,21-dioxa-bicycle[15.3.1]henicosa-9,19-dien-5-one. The TBS ether (1.8 mg, 0.0028 mmol) was dissolved in CH₃CN (0.2 mL) and H₂SiF₆ (1 drop of a 20-25% wt aqueous solution) was added. Typical aqueous workup and chromatography produced 1.5 mg of product. Spectral data confirmed the structure of the product.

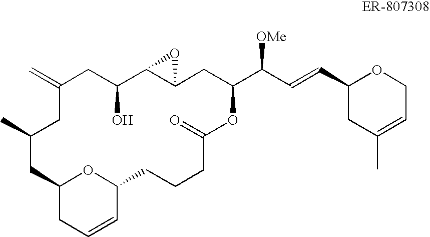

7-Hydroxy-12-[1-methoxy-3-(4-methyl-3,6-dihydro-2H-pyran-2-yl)-allyl]-3-methyl-5-methylene-9,13,22-trioxa-tricyclo[16.3.1.08,10]docos-19-en-14-one. A stock solution was made as follows: (+)-DIPT (87 4, 97 mg, 0.41 mmol) was dissolved in CH₂Cl₂ (1 mL), cooled to −40° C., Ti(OiPr)₄ (98 μL, 94 mg, 0.33 mmol) was added, the mixture stirred 5 min, then t-BuOOH (90 μL of a ˜5.5M solution in nonane, 0.50 mmol) was added and the solution stirred for an additional 15 min. The diol (1.5 mg, 0.0029 mmol) was dissolved in CH₂Cl₂ (0.3 mL), cooled to −20° C., and portions of the stock solution were added until the reaction was complete. Typical aqueous workup and chromatography produced 0.44 mg of product. Spectral data confirmed the structure of the product. MS (API, ESP+) m/z 553 [M+Na]⁺.

### Example 12

### Preparation of Diol Side Chain Compound

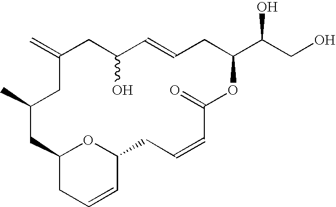

Exemplary Synthesis:

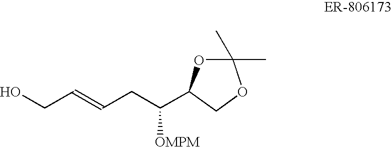

5-(2,2-Dimethyl-[1,3]dioxolan-4-yl)-5-(4-methoxy-benzyloxy)-pent-2-en-1-ol. Made in an analogous manner to ER-805262 starting from L-arabinose. Spectral data confirmed the structure of the product.

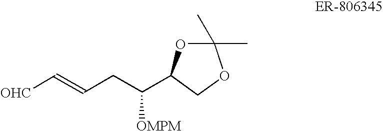

5-(2,2-Dimethyl-[1,3]dioxolan-4-yl)-5-(4-methoxy-benzyloxy)-pent-2-enal. Made in an analogous manner to ER-807910. Spectral data confirmed the structure of the product.

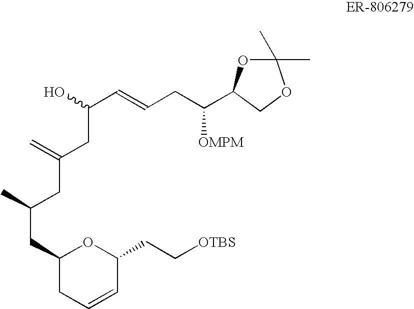

10-{6-[2-(tert-Butyl-dimethyl-silanyloxy)-ethyl]-3,6-dihydro-2H-pyran-2-yl}-1-(2,2-dimethyl-[1,3]dioxolan-4-yl)-1-(4-methoxy-benzyloxy)-9-methyl-7-methylene-dec-3-en-5-ol. Made in an analogous manner to ER-807302. Spectral data confirmed the structure of the product.

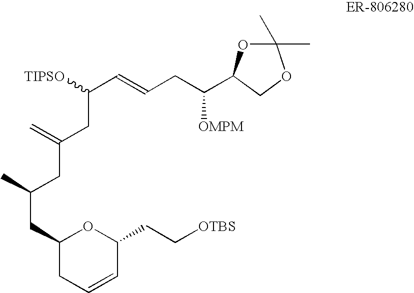

6-[2-(tert-Butyl-dimethyl-silanyloxy)-ethyl]-2-[10-(2,2-dimethyl-[1,3]dioxolan-4-yl)-10-(4-methoxy-benzyloxy)-2-methyl-4-methylene-6-triisopropylsilanyloxy-dec-7-enyl]-3,6-dihydro-2H-pyran. Made in an analogous manner to ER-808320. Spectral data confirmed the structure of the product.

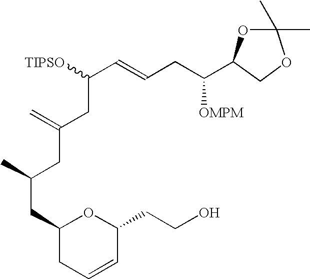

2-{6-[10-(2,2-Dimethyl-[1,3]dioxolan-4-yl)-10-(4-methoxy-benzyloxy)-2-methyl-4-methylene-6-triisopropylsilanyloxy-dec-7-enyl]-5,6-dihydro-2H-pyran-2-yl}-ethanol. The TBS ether (61 mg, 0.075 mmol) was dissolved a 1/1/1 mixture of HOAc/THF/H₂O (8 mL). Typical aqueous workup and chromatography produced 28 mg of product, 10 mg of recovered starting material, and 4 mg of diol. Spectral data confirmed the structure of the product.

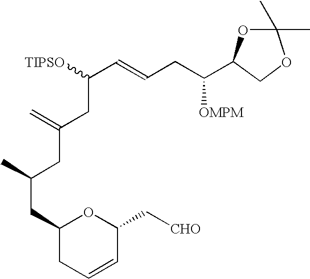

{6-[10-(2,2-Dimethyl-[1,3]dioxolan-4-yl)-10-(4-methoxy-benzyloxy)-2-methyl-4-methylene-6-triisopropylsilanyloxy-dec-7-enyl]-5,6-dihydro-2H-pyran-2-yl}-acetaldehyde. Made in an analogous manner to ER-809530. Spectral data confirmed the structure of the product.

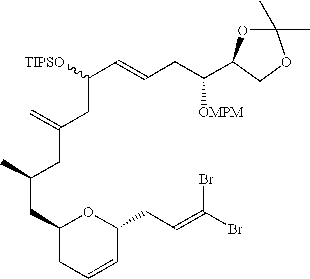

{6-[6-(3,3-Dibromo-allyl)-3,6-dihydro-2H-pyran-2-yl]-1-[4-(2,2-dimethyl-[1,3]dioxolan-4-yl)-4-(4-methoxy-benzyloxy)-but-1-enyl]-5-methyl-3-methylene-hexyloxy}-triisopropyl-silane. Made in an analogous manner to ER-809531. Spectral data confirmed the structure of the product.

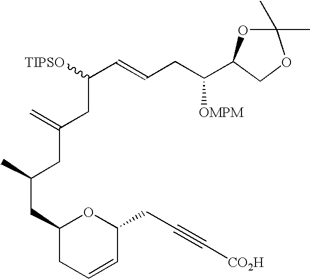

4-{6-[10-(2,2-Dimethyl-[1,3]dioxolan-4-yl)-10-(4-methoxy-benzyloxy)-2-methyl-4-methylene-6-triisopropylsilanyloxy-dec-7-enyl]-5,6-dihydro-2H-pyran-2-yl}-but-2-ynoic acid. Made in an analogous manner to ER-807320 using excess n-BuLi and quenching with CO₂ (gas or solid). Spectral data confirmed the structure of the product.

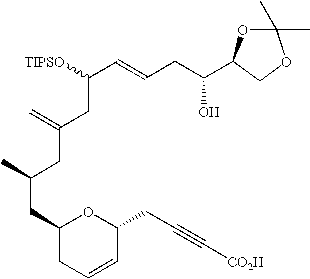

4-{6-[10-(2,2-Dimethyl-[1,3]dioxolan-4-yl)-10-hydroxy-2-methyl-4-methylene-6-triisopropylsilanyloxy-dec-7-enyl]-5,6-dihydro-2H-pyran-2-yl}-but-2-ynoic acid. Made in an analogous manner to ER-809583. Spectral data confirmed the structure of the product.

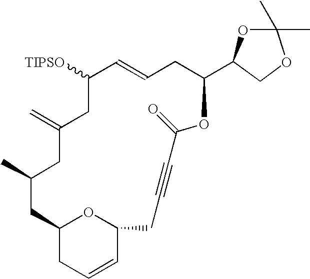

7-(2,2-Dimethyl-[1,3]dioxolan-4-yl)-15-methyl-13-methylene-11-triisopropyl silanyloxy-6,21-dioxa-bicyclo[15.3.1]henicosa-9,19-dien-3-yn-5-one. The alkynoic acid (24 mg, 0.038 mmol) was dissolved in benzene (2 mL), and Ph₃P (31 mg, 0.12 mmol) and DEAD (18 μL, 20 mg, 0.12 mmol) were added. Typical aqueous workup and chromatography produced 10 mg of product. Spectral data confirmed the structure of the product.

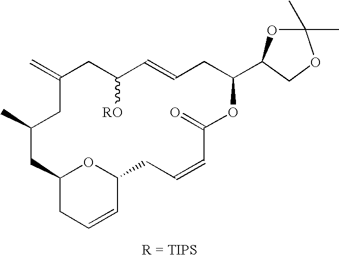

7-(2,2-Dimethyl-[1,3]dioxolan-4-yl)-15-methyl-13-methylene-11-triisopropyl silanyloxy-6,21-dioxa-bicyclo[15.3.1]henicosa-3,9,19-trien-5-one. Made in an analogous manner to ER-808426. Spectral data confirmed the structure of the product.

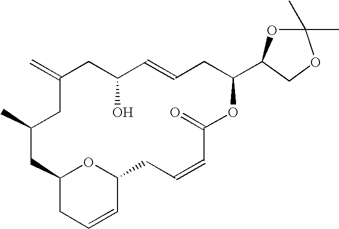

7-(2,2-Dimethyl-[1,3]dioxolan-4-yl)-11-hydroxy-15-methyl-13-methylene-6,21-dioxa-bicyclo[15.3.1]henicosa-3,9,19-trien-5-one. The TIPS ether (4.5 mg, 0.007 mmol) was dissolved in THF (1 mL), cooled to 0° C., TBAF (11 μL, of a 1M solution in THF, 0.011 mmol) was added and the reaction was warmed to RT. Typical aqueous workup and chromatography provided 1.6 mg of product. Spectral data confirmed the structure of the product.

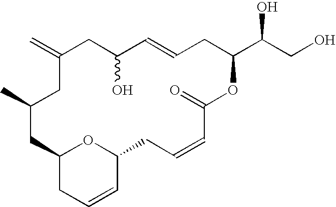

7-(1,2-Dihydroxy-ethyl)-11-hydroxy-15-methyl-13-methylene-6,21-dioxa-bicyclo[15.3.1]henicosa-3,9,19-trien-5-one. The TIPS ether acetonide (3.6 mg, 0.006 mmol) was dissolved in CH₃CN (0.5 mL), and H₂SiF₆ (1 drop of a 20-25 wt % solution) was added. Direct chromatography provided 2 mg of product. Spectral data confirmed the structure of the product.

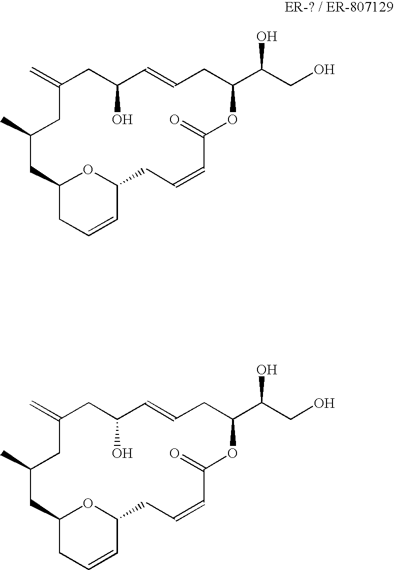

HPLC purification of the C.15 diastereomers. The mixture of diastereomers was separated by semi-prep HPLC using Chiralpak AD stationary phase and a 15% IPA/hexanes mobile phase. Spectral data confirmed the structures of the products.

### Example 13

### Laulimalide Analogues Derived from ER-807321 and ER-807910

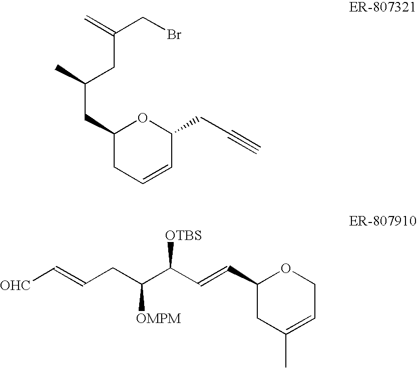

For an Exemplary Synthesis of diastereomers ER-807901 and 807903, see Example 8 (e.g., ER-808426).

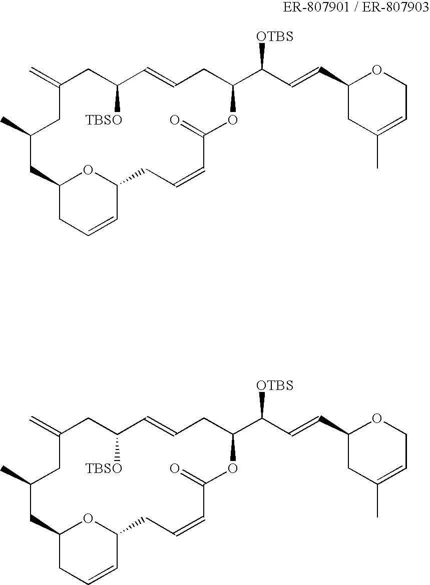

HPLC purification of the C.15 diastereomers. The mixture of diastereomers was separated by prep HPLC using Chiralpak AD stationary phase and a 1% IPA/hexanes mobile phase.

Des-epoxy-Laulimalide.

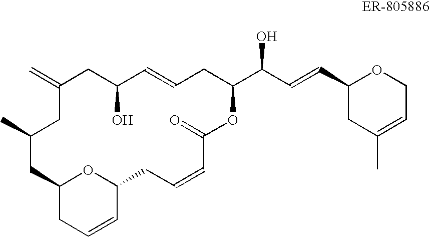

The bis-TBS compound (412 mg, 0.567 mmol) was dissolved in THF (32 mL), and HF/pyridine (12.33 mL of a 70% HF 30% pyridine solution) was added over 10 min. Neutralization, typical aqueous workup, and chromatography produced 273 mg of product. Spectral data confirmed the structure of the product. MS (API, ESP+) m/z 521 [M+Na]⁺.

(−)-Laulimalide.

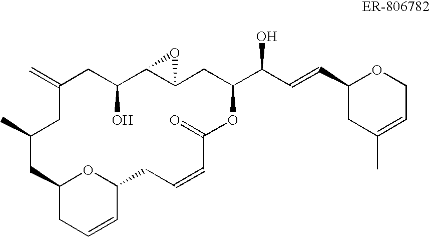

(+)-DIPT (0.84 mL, 0.94 g, 3.99 mmol) was dissolved in CH₂Cl₂ (100 mL), 4 Å molecular sieves (8.72 g) were added, and the mixture was cooled to −20° C. Ti(OiPr)₄ (0.99 mL, 0.95 g, 3.33 mmol) was added, the mixture stirred 5 min, then t-BuOOH (1.2 mL of a ˜5.5M solution in nonane, 6.6 mmol) was added and the reaction stirred for an additional 40 min. The diol (273 mg, 0.547 mmol) was dissolved in CH₂Cl₂ (30 mL) and added over ˜20 min to the reaction mixture at −20° C. Typical aqueous workup and chromatography produced 233 mg of product.

Spectral data confirmed the structure of the product. MS (API, ESP+) m/z 537 [M+Na]⁺.

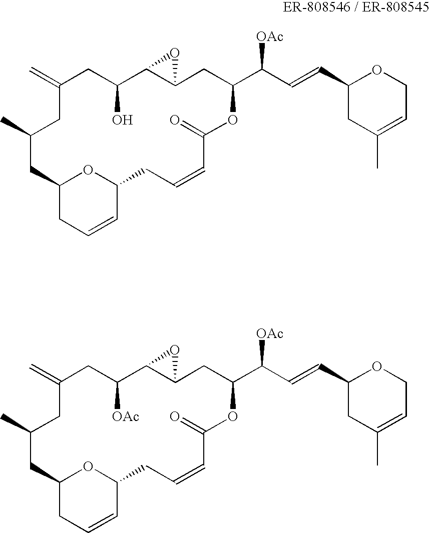

Acetic acid 1-(7-hydroxy-3-methyl-5-methylene-14-oxo-9,13,22-trioxa-tricyclo[16.3.1.08,10]docosa-15,19-dien-12-yl)-3-(4-methyl-3,6-dihydro-2H-pyran-2-yl)-allyl ester and acetic acid 12-[1-acetoxy-3-(4-methyl-3,6-dihydro-2H-pyran-2-yl)-allyl]-3-methyl-5-methylene-14-oxo-9,13,22-trioxa-tricyclo[16.3.1.08,10]docosa-15,19-dien-7-yl ester. The diol (1.3 mg, 0.0025 mmol) was dissolved in pyridine (1 mL), acetic anhydride (25 μL, 27 mg, 0.26 mmol) and DMAP (a crystal) were added. Typical aqueous workup and chromatography provided 0.3 mg of the mono-acetate product and 0.8 mg of the bis-acetate product. Spectral data confirmed the structures of both products. MS of ER-808546 (API, ESP+) m/z 579 [M+Na]⁺. MS of ER-808545 (API, ESP+) m/z 621 [M+Na]⁺.

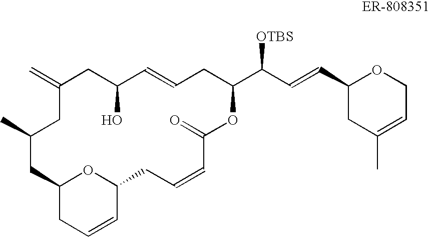

7-[1-(tert-Butyl-dimethyl-silanyloxy)-3-(4-methyl-3,6-dihydro-2H-pyran-2-yl)-allyl]-11-hydroxy-15-methyl-13-methylene-6,21-dioxa-bicyclo[15.3.1]henicosa-3,9,19-trien-5-one. The bis-TBS ether (125 mg, 0.17 mmol) was dissolved in a 2/2/1 mixture of CH₂Cl₂/CH₃CN/THF (25 mL), cooled to 0° C., and H₂SiF₆ (0.35 mL of a 20-25% wt aqueous solution) was added. Typical aqueous workup and chromatography produced 75 mg of product, as well as recovered starting material and fully deprotected products. Spectral data confirmed the products. MS of ER-808351 (API, ESP+) m/z 635 [M+Na]⁺.

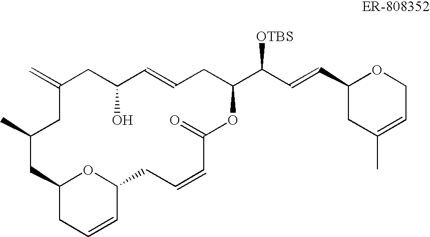

7-[1-(tert-Butyl-dimethyl-silanyloxy)-3-(4-methyl-3,6-dihydro-2H-pyran-2-yl)-allyl]-11-hydroxy-15-methyl-13-methylene-6,21-dioxa-bicyclo[15.3.1]henicosa-3,9,19-trien-5-one. The bis-TBS ether (478 mg, 0.657 mmol) was dissolved in a 2/2/1 mixture of CH₂Cl₂/CH₃CN/THF (60 mL), cooled to 0° C., and H₂SiF₆ (1.65 mL of a 20-25% wt aqueous solution) was added. Typical aqueous workup and chromatography produced 250 mg of product, as well as recovered starting material and fully deprotected products. Spectral data confirmed the product. MS (API, ESP+) m/z 635 [M+Na]⁺.

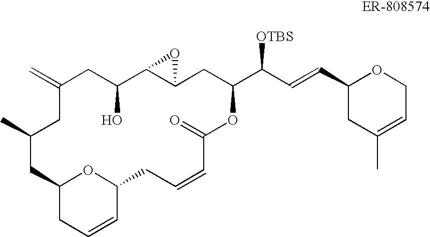

12-[1-(tert-Butyl-dimethyl-silanyloxy)-3-(4-methyl-3,6-dihydro-2H-pyran-2-yl)-allyl]-7-hydroxy-3-methyl-5-methylene-9,13,22-trioxa-tricyclo[16.3.1.08,10]docosa-15,19-dien-14-one. (+)-DIPT (395 μL of a 1M solution in CH₂Cl₂, 395 mmol) was dissolved in CH₂Cl₂ (12 mL), 4 Å molecular sieves (0.89 g) were added, and the mixture was cooled to −20° C. Ti(OiPr)₄ (95) μL, 91 mg, 0.32 mmol) was added, the mixture stirred 5 min, then t-BuOOH (120 μL, of a ˜5.5M solution in nonane, 0.66 mmol) was added and the reaction stirred for an additional 40 min. The diol (33 mg, 0.054 mmol) was dissolved in CH₂Cl₂ (6 mL) and added over ˜20 min to the reaction mixture at −20° C. Typical aqueous workup and chromatography produced 33 mg of product. Spectral data confirmed the structure of the product.

(−)-Laulimalide.

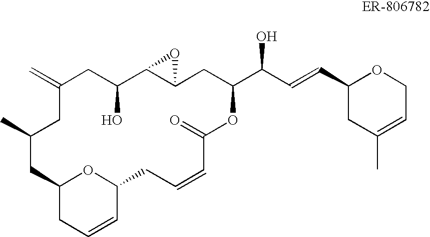

As reported by Crimmins et al. J. Am. Chem. Soc. 2002, 124, 5958, the TBS ether (43 mg, 0.068 mmol) was dissolved in CH₃CN (5 mL) and EtN₃-3HF (˜1.1 g) was added dropwise. Typical aqueous workup and chromatography produced 8.8 mg of product. Spectral data confirmed the structure of the product. MS (API, ESP+) m/z 537 [M+Na]⁺.

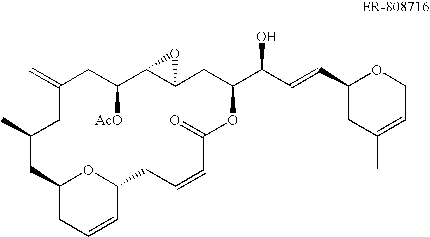

Acetic acid 12-[1-hydroxy-3-(4-methyl-3,6-dihydro-2H-pyran-2-yl)-allyl]-3-methyl-5-methylene-14-oxo-9,13,22-trioxa-tricyclo[16.3.1.08,10]docosa-15,19-dien-7-yl ester. The alcohol (2.3 mg, 0.0037 mmol) and acetic anhydride (50 μL, 54 mg, 0.53 mmol) were dissolved in pyridine (0.5 mL). Typical aqueous workup produced the crude product. The crude product was dissolved in CH₃CN (0.5 mL), and NEt₃-3HF (100 mg) was added. Typical aqueous workup and chromatography produced 0.68 mg of product. Spectral data confirmed the structure of the product.

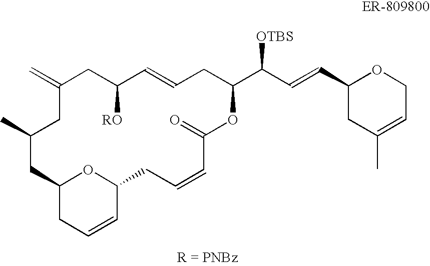

4-Nitro-benzoic acid 7-[1-(tert-butyl-dimethyl-silanyloxy)-3-(4-methyl-3,6-dihydro-2H-pyran-2-yl)-allyl]-15-methyl-13-methylene-5-oxo-6,21-dioxa-bicyclo[15.3.1]henicosa-3,9,19-trien-11-yl ester. The alcohol (250 mg, 0.407 mmol) was dissolved in toluene (10 mL), cooled to 0° C., and Ph3P (0.32 g. 1.20 mmol) and p-nitrobenzoic acid (204 mg, 1.22 mmol) were added, followed by dropwise addition of DEAD (0.19 mL, 0.21 g, 1.21 mmol). The reaction was warmed to RT, and typical aqueous workup and chromatography provided 191 mg of product. Spectral data confirmed the structure of the product.

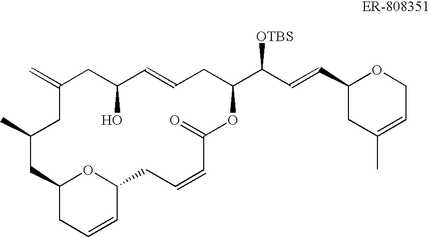

7-[1-(tert-Butyl-dimethyl-silanyloxy)-3-(4-methyl-3,6-dihydro-2H-pyran-2-yl)-allyl]-11-hydroxy-15-methyl-13-methylene-6,21-dioxa-bicyclo[15.3.1]henicosa-3,9,19-trien-5-one. The p-nitrobenzoate (271 mg, 0.33 mmol) was dissolved in MeOH (13 mL), and K₂CO₃ (32 mg, 0.24 mmol) was added. Typical aqueous workup and chromatography produced 174 mg of product. Spectral data confirmed the product. MS (API, ESP+) m/z 635 [M+Na]⁺.

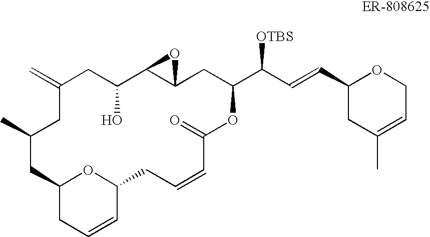

12-[1-(tert-Butyl-dimethyl-silanyloxy)-3-(4-methyl-3,6-dihydro-2H-pyran-2-yl)-allyl]-7-hydroxy-3-methyl-5-methylene-9,13,22-trioxa-tricyclo[16.3.1.08,10]docosa-15,19-dien-14-one. Made in an analogous manner to ER-808574 using (−)-DIPT. Spectral data confirmed the structure of the product.

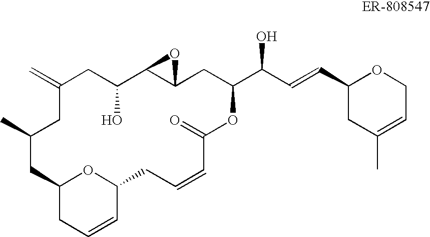

7-Hydroxy-12-[1-hydroxy-3-(4-methyl-3,6-dihydro-2H-pyran-2-yl)-allyl]-3-methyl-5-methylene-9,13,22-trioxa-tricyclo[16.3.1.0^{8.10}]docosa-15,19-dien-14-one. Made in an analogous manner to ER-806782. Spectral data confirmed the structure of the product.

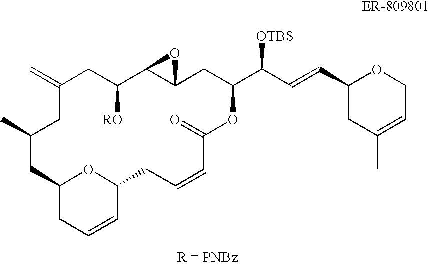

4-Nitro-benzoic acid 12-[1-(tert-butyl-dimethyl-silanyloxy)-3-(4-methyl-3,6-dihydro-2H-pyran-2-yl)-allyl]-3-methyl-5-methylene-14-oxo-9,13,22-trioxa-tricyclo[16.3.1.0^{8,10}]docosa-15,19-dien-7-yl ester. Made in an analogous manner to ER-809800. Spectral data confirmed the structure of the product.

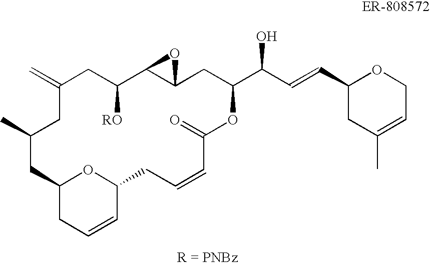

4-Nitro-benzoic acid 12-[1-hydroxy-3-(4-methyl-3,6-dihydro-2H-pyran-2-yl)-allyl]-3-methyl-5-methylene-14-oxo-9,13,22-trioxa-tricyclo[16.3.1.0^{8,10}]docosa-15,19-dien-7-yl ester. Made in an analogous manner to ER-808716. Spectral data confirmed the structure of the product.

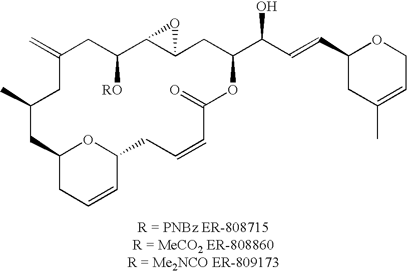

Acylated analogs of C.15 hydroxyl. Made in an analogous manner to ER-808716. Spectral data confirmed the structure of all products.

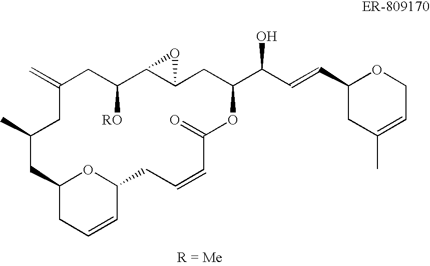

12-[1-Hydroxy-3-(4-methyl-3,6-dihydro-2H-pyran-2-yl)-allyl]-7-methoxy-3-methyl-5-methylene-9,13,22-trioxa-tricyclo[16.3.1.0^{8,10}]docosa-15,19-dien-14-one. The alcohol (1 mg, 0.002 mmol) was dissolved in THF (0.5 mL), NaH (0.8 mg of a 60% dispersion, 0.02 mmol) was added, followed by MeI (2.8 mg, 0.02 mmol). Typical aqueous workup and chromatography provided the product. Spectral data confirmed the structure of the product. The TBS group was removed in a manner analogous to ER-806782 to produce 0.5 mg of product. Spectral data confirmed the structure of the product.

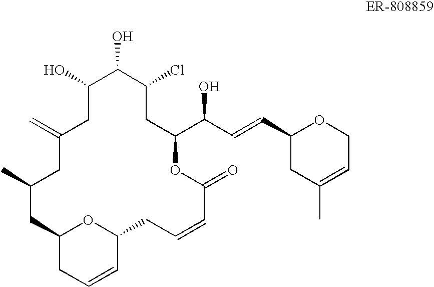

9-Chloro-10,11-dihydroxy-7-[1-hydroxy-3-(4-methyl-3,6-dihydro-2H-pyran-2-yl)-allyl]-15-methyl-13-methylene-6,21-dioxa-bicyclo[15.3.1]henicosa-3,19-dien-5-one. The alcohol ER-808574 (7.0 mg, 0.011 mmol) was dissolved in pyridine (0.8 mL), dimethylcarbamyl chloride (20 μL, 23 mg, 0.22 mmol) was added, and the reaction was heated to reflux. Additional dimethylcarbamyl chloride (100 μL) was added to completely consume the starting material. Typical aqueous workup and chromatography produced 3 mg of product. Spectral data continued the structure of the product. MS (API, ESP+) m/z 687 [M+Na]⁺. The TBS group was removed in a manner analogous to ER-806782. Spectral data confirmed the structure of the product.

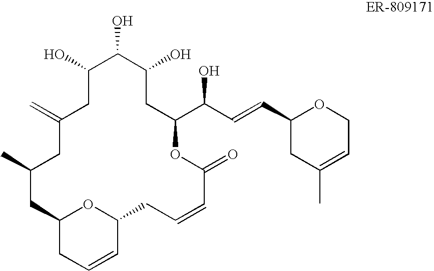

9,10,11-Trihydroxy-7-[1-hydroxy-3-(4-methyl-3,6-dihydro-2H-pyran-2-yl)-allyl]-15-methyl-13-methylene-6,21-dioxa-bicyclo[15.3.1]henicosa-3,19-dien-5-one. The alcohol ER-808574 (5.0 mg, 0.008 mmol) was dissolved in THF (0.5 mL), NaH (3.2 mg of a 60% dispersion, 0.080 mmol) was added, followed by MOMCl (6.4 mg, 0.080 mmol). Typical aqueous workup and chromatography produced 1 mg of product. Spectral data confirmed the structure of the product. MS (API, ESP+) m/z 669 [M+Na]⁺. The TBS group was removed in a manner analogous to ER-806782 to produce 1 mg of product. Spectral data confirmed the structure of the product.

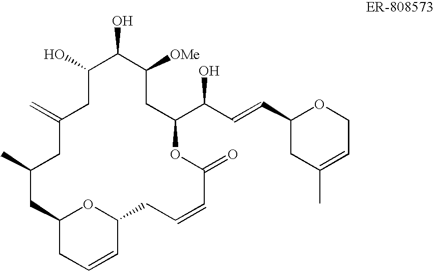

10,11-Dihydroxy-7-[1-hydroxy-3-(4-methyl-3,6-dihydro-2H-pyran-2-yl)-allyl]-9-methoxy-15-methyl-13-methylene-6,21-dioxa-bicyclo[15.3.1]henicosa-3,19-trien-5-one. The PNBz ester ER-808572 (1.0 mg, 0.002 mmol) was dissolved in MeOH (0.5 mL) and 1M aqueous NaOH (0.25 mL). Typical aqueous workup and chromatography produced 0.5 mg of product. Spectral data confirmed the structure of the product. MS (API, ESP+) m/z 569 [M+Na]⁺.

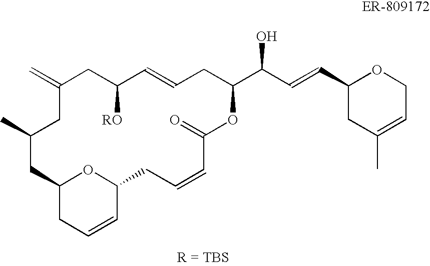

11-(tert-Butyl-dimethyl-silanyloxy)-7-[1-hydroxy-3-(4-methyl-3,6-dihydro-2H-pyran-2-yl)-allyl]-15-methyl-13-methylene-6,21-dioxa-bicyclo[15.3.1]henicosa-3,9,19-trien-5-one. This product was formed as a minor bi-product during the deprotection of ER-807901 to yield ER-808351. Spectral data confirmed the structure of the product.

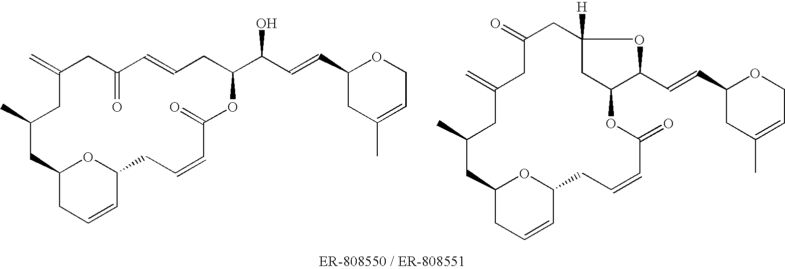

7-[1-Hydroxy-3-(4-methyl-3,6-dihydro-2H-pyran-2-yl)-allyl]-15-methyl-13-methylene-6,21-dioxa-bicyclo[15.3.1]henicosa-3,9,19-triene-5,11-dione and 16-methyl-8-[2-(4-methyl-3,6-dihydro-2H-pyran-2-yl)-vinyl]-14-methylene-6,9,22-trioxa-tricyclo[16.3.1.1^{7,10}]tricosa-3,20-diene-5,12-dione. The alcohol ER-808352 (5 mg, 0.008 mmol) was dissolved in CH₂Cl₂ (0.5 mL), cooled to 0° C., and Dess-Martin periodane (4.2 mg, 0.010 mmol) was added. Typical aqueous workup and chromatography produced 5 mg of TBS protected enone. The TBS group was removed in an analogous manner to ER-806782 to produce 1.2 mg of ER-808550 and 1.5 mg of ER-808551. Spectral data confirmed the structures of the products.

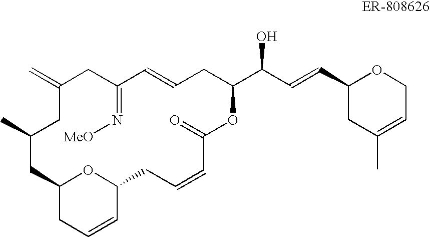

7-[1-Hydroxy-3-(4-methyl-3,6-dihydro-2H-pyran-2-yl)-allyl]-15-methyl-13-methylene-6,21-dioxa-bicyclo[15.3.1]henicosa-3,9,19-triene-5,11-dione 11-(O-methyl-oxime). The TBS protected enone (3 mg, 0.005 mmol) was dissolved in MeOH (0.3 mL) and methoxyamine hydrochloride (2 mg, 0.02 mmol) was added. Typical aqueous workup produced the crude product. The TBS group was removed in an analogous manner to ER-806782 to produce 1 mg, of product. Spectral data confirmed the structure of the product.

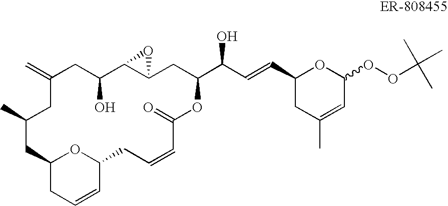

12-[3-(6-tert-Butylperoxy-4-methyl-3,6-dihydro-2H-pyran-2-yl)-1-hydroxy-allyl]-7-hydroxy-3-methyl-5-methylene-9,13,22-trioxa-tricyclo[16.3.1.0^{8,10}]docosa-15,19-dien-14-one. This was a side-product during the final step of the synthesis of ER-806782. Spectral data confirmed the structure of the product as a mixture of diastereomers. MS (API, ESP+) m/z 625 [M+Na]⁺.

### Example 14

### Additional Laulimalide Analogues

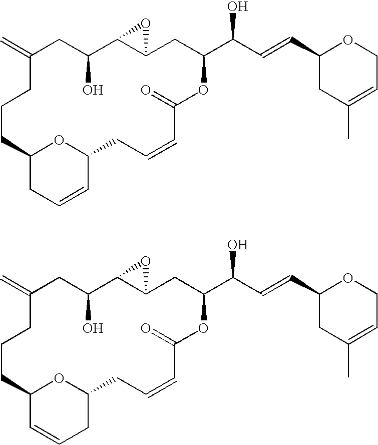

7-Hydroxy-12-[1-hydroxy-3-(4-methyl-3,6-dihydro-2H-pyran-2-yl)-allyl]-5-methylene-9,13,22-trioxa-tricyclo[16.3.1.0^{8,10}]docosa-15,19-dien-14-one or 7-hydroxy-12-[1-hydroxy-3-(4-methyl-3,6-dihydro-2H-pyran-2-yl)-allyl]-5-methylene-9,13,22-trioxa-tricyclo[16.3.1.0^{8,10}]docosa-15,20-dien-14-one. These compounds can be made in a manner analogous to ER-807331 by reductive removal of the C.8 acetate utilizing the following procedure:

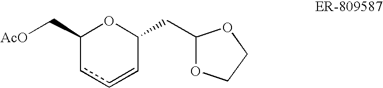

Acetic acid 6-[1,3]dioxolan-2-ylmethyl-3,6-dihydro-2H-pyran-2-ylmethyl ester and acetic acid 6-[1,3dioxolan-2-ylmethyl-5,6-dihydro-2H-pyran-2-ylmethyl ester. Pd₂(dba)₃CHCl₃ (72 mg, 0.069 mmol) was dissolved in dioxane (12 mL), Bu₃P (72 mL 58 mg, 0.29 mmol) was added, the reaction was heated to 70° C., and a solution of the his-acetate ER-805261 (1.03 g, 3.43 mmol) in dioxane (8 mL) was added. Typical aqueous workup and chromatography provided 0.55 g of product. Spectral data confirmed the structure of the product as a ˜2:1 mixture of double bond isomers.

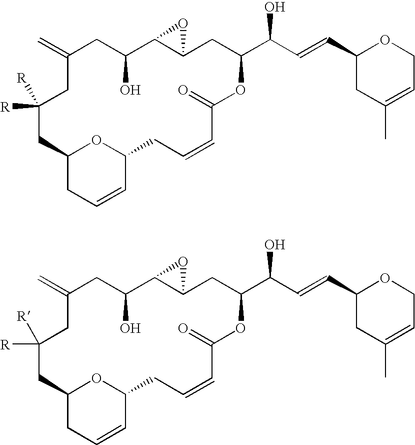

C.11 Substituted Analogs. Compounds of this type can be made in a manner analogous to ER-807331 by performing conjugate additions via a cuprate reagent) to an ynone analogous to ER-806639. See for examples, J. A. Marshall et al. J. Org. Chem. 1990, 55, 227 or A. B. Dounay and C. J. Forsyth Org. Letters 1999, 1, 451.

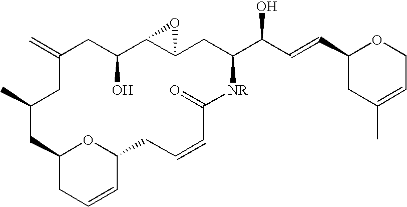

Lactam Derivatives, e.g. R═H 7-hydroxy-12-[1-hydroxy-3-(4-methyl-3,6-dihydro-2H-pyran-2-yl)-allyl]-3-methyl-5-methylene-9,22-dioxa-13-aza-tricyclo[16.3.1.0^{8,10}]docosa-15,19-dien-14-one. Lactam derivatives may be prepared through a macrolactamization protocol between the appropriate C.19 substituted amine and C.1 acid. See for examples, A. B. Smith III et al. Org. Letters 1999, 1, 1491, R. M. Borzelleri et al. J. Am. Chem. Soc. 2000, 122, 8890, or K. C. Nicolaou et al. Angew. Chem. Int. Ed. 2002, 41, 1937. The C.19 amine or protected amine can be prepared from the corresponding C.19 alcohol in a number of previously described intermediates.

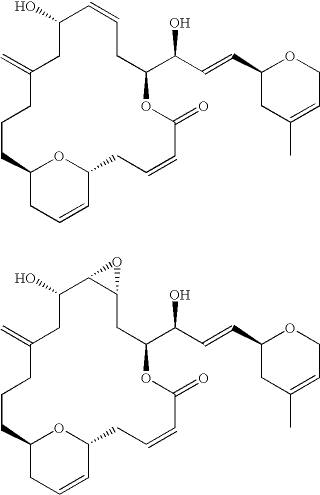

C.16-C.17 cis Alkene and cis Epoxide Derivatives. Compounds of this type may be prepared by performing a Z selective olefination of the aldehyde prepared from ER-807932 (see ER-806341 prep.) by the methods of W. C. Still and C. Gennari Tetrahedron Lett. 1983, 24, 4405 or K. Ando J. Org. Chem. 1998, 63, 8411.

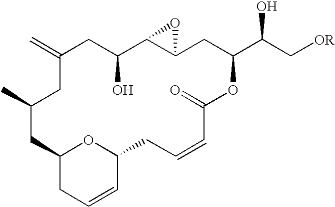

C.21 Substituted Compounds. Compounds of this type may be produced through selective alkylation or acylations of the primary hydroxyl by one of the many published procedures. See for examples, R. D. Walkup et al. Tetrahedron Lett. 1987, 28, 4019 or N. Nagashima et al. Chem. Pharm. Bull. 1991, 39, 1972 or T. Ogawa et al. Tetrahedron 1981, 2363.

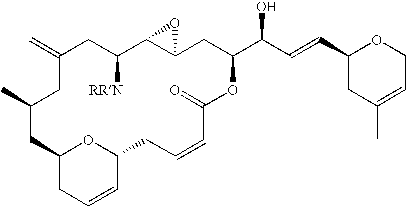

C.15 N-Substituted Compounds. Compounds of this type can be produced by displacement of a suitable leaving group with a nitrogen nucleophile or through a Mitsunobu-type reaction of compounds analogous to ER-808351, ER-808352, ER-808574, etc. See for examples, W. H. Pearson et al. J. Org. Chem. 1989, 54, 4235 or D. J. Hart J. Am. Chem. Soc. 1980, 102, 397.

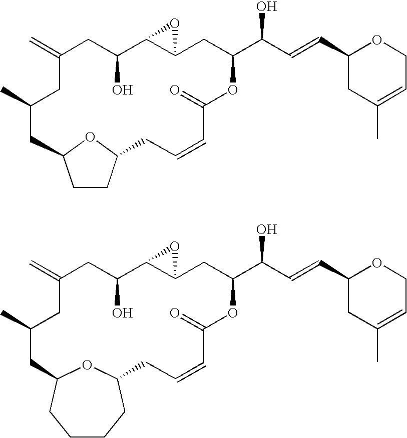

Furanyl-type and Oxepane-type Derivatives. These compounds can be prepared in an analogous manner to the pyran compounds, e.g. ER-806407. For examples see, C. S. Wilcox et al. Tetrahedron Lett. 1986, 27, 1011 or F. P. J. T. Rutjes et al. Synlett 1998, 192.

2) Biological Data:

Cell Growth Inhibition Assays:

The cancer cell growth inhibition assays were performed following the procedure described by Towle et al. (Cancer Research, 2001, 61(3):1013-1021). Cells were plated at 7.5×10³ cells/well into 96-well plate with 100 μl of cell culture medium. After 4-5 hours incubation at 37° C., 100 μl of 2× test compounds was added over the cell. After incubation for 3-4 days, the medium was removed with an aspirating pipe attached with a tip. Then 100 μl of methylene blue (5 mg/ml in 50% EtOH) was added to each well and incubated for 30 minutes. The dye was shaken away and the plate was washed 4 times with running water (submerged in to the water without emptying the well. The plate was air-dried and then 100 μl of sarcosine (10 mg/ml in PBS) was added to each well. The plate was shaken for 1-2 hours at room temperature. The plate was read on Titertek Multixcan MCC340 at A₆₀₀-A₄₀₅.

Growth Inhibition Assay Procedure

The cells were seeded at 7.5×10³ cells/well in DMEM (for MDA-MB-435) or McCoy's 5A (for HT-29) supplemented with 10% FBS and penicillin, streptomycin and L-glutamine. After 4 hours incubation, the test compound was added to each well to give a series of concentration ranging from 0 to 10 μM. The cultures were incubated for 4 days at 37° C. Then the medium was removed and the cells were stained with 100 μl of methylene blue (500 μg/ml) for 45 min. After wash with water, the stained cells were dissolved into 100 μl of sarcosine (1 mg/ml) for 90 min with gentle shaking. The plates were read at A₆₀₀-A₄₀₅.

Procedure for Determining Compound Stability in Mouse Serum

The compound was incubated in 100% mouse serum for 6 hours at 37° C. Then the compound was diluted and added to the cell culture with 1% of mouse serum. After 4 days incubation, the activity of the compound was determined as general growth inhibition assays.

Susceptibility of Compound to P-Glycoprotein-Mediated Multidrug Resistance (MDR).

A pair of human uterine sarcoma cell lines was used: MES-SA, the MDR negative parental cell line, and Dx5-Rx1 , a cell line derived from MES-SA after long term of exposure to doxorubicin. This subline expresses PgP at high levels. Both cell lines were seeded at 7.5×10³ cells/well in McCoy's 5A supplemented with 10% FBS and penicillin, streptomycin and L-glutamine. After 4 hours incubation, the test compound was added to each well to give a series of concentration ranging from 0 to 10 μM. The cultures were incubated for 4 days at 37° C. The medium was removed and the cells were stained with methylene blue (500 μg/ml) for 45 min. After wash with water, the stained cells were dissolved into 100 ml of sarcosine (1 mg/ml) for 90 min with gentle shaking. The plates were read at A₆₀₀-A₄₀₅. The IC₅₀ values against the two cell lines were compared with each other.

Cytotoxicity Assay Procedure

The human fibroblast IMR-90 cells were seeded at 1×10⁴ cells/well in MEM containing 10% FBS and penicillin, streptomycin and L-Glutamine and grown to 100% confluency at 37° C. The media was replaced with complete MEM containing 0.1% FBS and the cells were cultured for 3 days after which the compound was added at concentrations ranging from 0-10 μM. The cultures were incubated for 24 hours at 37° C. and ATP was measured as an indicator of cell viability using the ATPLite-M assay kit (Perkin Elmer).

Reversibility Assay Procedure

Set up 10 T75 flasks with 2.5×10⁶ U937 (lymphoma-monocyte-like) cells in 22.5 mls of RPMI 1640 medium containing 10% FBS, penicillin, streptomycin and L-glutamine.

Incubation for 36 hours, add 2.5 ml of 10× concentrations of the compound to each flask to give the final concentrations of 10,000 nM, 3,000 nM, 1000 nM, 300 nM, 100 nM, 30 nM, 10 nM, 3 nM, and 1 nM, respectively.

After incubation for 12 hours with compound, the cell culture was transferred into 50 ml tubes and centrifuged. After wash with 25 ml of medium, the cell pellet was resuspended into 35 ml of medium. 10 ml of the cells was used to fix and stain for flow cytometry (0 time point). The rest 15 ml cell culture was continued to grow additional 10 hours (for 10 hour time point).

Then 10 ml of cell culture was centrifuged and the cell pellet was resuspended into 3 ml of cold saline (0.9% NaCl). Then 7 ml of 100% ethanol was added to the cell suspension. The cell sample was stored at 4° C.

The fixed cells were spun at 1400 rpm for 10 minutes at room temperature and washed with 10 mls of PBS. The cell pellet was resuspended into 0.5 ml of RNase A (0.2 mg/ml) and incubated in 37° C. water bath for 30 minutes.

The cell samples were stained with 0.5 ml PI (Propidium iodide, 10 ug/ml) and analyzed by flow cytometry.

Additional Assays:

Additional guidance regarding exemplary assays useful for testing compounds of the invention may be found in Towle et al., “In Vitro and In Vivo Anticancer Activities of Synthetic Macrocyclic Ketone Analogues of Halichondrin B”, Cancer Research, 2001, 61(3):1013-1021); which is incorporated herein by reference in its entirety.

The following describes exemplary assays to assess the microtubule stabilizing ability of compounds of the invention. In certain embodiments, the assays may assess the ability of inventive compounds to inhibit the proliferation of a hyperproliferative mammalian cell having a multiple drug resistant phenotype. Examples of these assays are described in published US application 2002/0198256; paragraphs [0057]-[0098] of which are hereby incorporated herein by reference.

Reagents

The various assays may be performed using a variety of reagents, including 4,6-Diamidino-2-phenylindole (DAPI), sulforhodamine B (SRB), antibodies against .beta.-tubulin, Basal Medium Eagle containing Earle's salts (BME), Richter's medium and Fetal Bovine Serum (FBS).

4,6-Diamidino-2-phenylindole (DAPI), sulforhodamine B (SRB), antibodies against .beta.-tubulin, and Basal Medium Eagle containing Earle's salts (BME) may be obtained from the Sigma Chemical Company (St. Louis, Mo.). Richter's medium may be obtained from BioWhittaker (Walkersville, Md.) and Fetal Bovine Serum (FBS) may be obtained from Hyclone Laboratories (Logan, Utah).

Cell Lines

The A-10 rat aortic smooth muscle and SK-OV-3 human ovarian carcinoma cell lines may be used to assess compounds activities. The A-10 rat aortic smooth muscle and SK-OV-3 human ovarian carcinoma cell lines may be obtained from the American Type Culture Collection (Manassas, Va.). For example, the A-10 rat aortic smooth muscle and SK-OV-3 human ovarian carcinoma cell lines may be cultured in BME containing 10% FBS and 50 .mu.g/mL gentamycin sulfate. A sub-line of SK-OV-3 selected for resistance to vinblastine (SKVLB-1) may be provided by Dr. Victor Ling (British Columbia Cancer Center, Vancouver, British Columbia) and may be maintained in BME containing 10% FBS and 50 μg/mL gentamycin sulfate. The MDA-MB-435 human mammary adenocarcinoma cell line may be obtained from Dr. Mai Higazi (Georgetown University, Washington, D.C.), and may be maintained in Richters medium containing 10% FBS and 50 μg/mL gentamycin sulfate. Vinblastine may be added to a final concentration of 1 .μg/mL to SKVLB-1 cells 24 hours after passage to maintain selection pressure for P-glycoprotein-overexpressing cells.

Inhibition of Cell Proliferation

The IC₅₀ values for inhibition of cell proliferation may be determined by measuring cell-associated protein after drug treatment using the sulforhodamine B assay.

Immunofluorescence Assays

A-10 cells may be grown to 70-85% confluence on glass coverslips in BME supplemented with 10% FBS. Drug compounds in PBS may be added to the indicated final concentrations and cells may be incubated for an additional 24 hours.

For the staining of microtubules and intermediate filaments, the cells may be fixed with cold methanol for 5 minutes, blocked for 20 minutes with PBS containing 10% calf serum to block nonspecific binding sites, and incubated at 37° C. for 90 min with monoclonal anti-β-tubulin at the dilutions recommended by the manufacturer. Bound primary antibodies may be subsequently visualized by a one hour incubation with fluorescein(FITC)-conjugated sheep antimouse IgG (F-3008; Sigma). The coverslips may be washed, stained with 0.1 μg/mL DAPI for 10 minutes, mounted on microscope slides and the fluorescence patterns may be examined and photographed using a Zeiss Axioplan microscope equipped with epifluorescence optics for fluorescein and DAPI.

Effects of Inventive Compounds on Cellular Microtubules.

A-10 cells may be treated with an inventive compound for 18 hours and the morphological effects on microtubules may be examined by indirect immunofluorescence techniques. The control cells exhibit normal microtubules arrays with filamentous microtubules radiating from the microtubule organizing center to the cell periphery. Treatment of the cells with an inventive compound disrupts the normal microtubule array; the microtubules are more numerous and appear to occupy more of the cytoplasm.

Effects of Inventive Compounds on Nuclear Structure.

A-10 and SK-OV-3 cells treated with a wide range of concentrations of inventive compounds exhibit the formation of multiple micronuclei. The effects of inventive compounds on nuclear structure may be visible: The normal rounded shape of the nucleus, which is devoid of microtubules, can be detected, whereas in compound-treated cells this distinct microtubule-free area containing the discrete central nucleus is lost and only vesicle-like areas devoid of microtubules remain. Typically, but not necessarily, nuclear staining of control cells reveal a central compact nucleus, whereas compound-treated cells exhibit a dramatic breakdown of the nucleus into micronuclei.

Effects of Inventive Compounds on Cell Cycle Progression and Mitotic Spindles.

A common characteristic of anti-microtubule agents is their ability to initiate mitotic arrest. Compounds of the invention may be assessed for their ability to disrupt microtubule dynamics, prevent normal mitotic progression and lead to mitotic arrest. Flow cytometric analysis may be used to perform the assay. For example, MDA-MB-435 breast carcinoma cells may be treated with various concentrations of inventive compounds within nine hours of treatment. Cell cycle arrest in G₂-M may be observed.

Effects of Inventive Compounds on Cell Proliferation of Drug-Sensitive and Multidrug-Resistant Cell Lines.

For example, experiments may be conducted to determine the IC₅₀ values for inventive compounds in two drug-sensitive cell lines, MDA-MB-435 and SK-OV-3, and in a multidrug-resistant cell line, SKVLB-1. Cells may be treated with varying concentrations of the compounds for 48 hours, and cell-associated protein may be determined using the SRB assay. The IC₅₀ value for each compound may be calculated for each cell line.

The IC₅₀ for inhibition of proliferation may also be determined in the A-10 cell line, a nontransformed line that may be used to show the effects of microtubule-stabilizing agents on cellular structures.

Initiation of Apoptosis by Inventive Compounds.

The ultimate mechanism of action of many cytotoxic cancer chemotherapeutic agents is the initiation of pathways of gene and protein expression leading to apoptosis. Antimicrotubule drugs including paclitaxel, vinblastine, and cryptophycin 1 initiate apoptosis both in vitro and in vivo. Flow cytometry data may be used to assess initiation of apoptosis. The loss of cellular DNA is detected by the appearance of the subdiploid peak when apoptotic cells are analyzed by flow cytometry.

During apoptosis, specific cysteine proteases called the caspases are activated. Activation of the caspase cascade leads to the proteolytic degradation of specific cellular proteins. The activation of caspase 3 and the proteolysis of the DNA repair enzyme PARP, a downstream substrate of caspase 3, are examined in cell lysates from compound-treated cells. For example, activation of caspase 3 leads to the loss of the 32 kDa proenzyme and the formation of the activation products p17 and p12. Analysis of immunoblot data from cell lysates can show the formation of the p17 activation product and/or the p32 proenzyme after compound treatment. The specific proteolysis of PARP by caspase 3 leads to the formation of two products, an 89 kDa COOH-terminal fragment and a 24 kDa N-terminal fragment. Observation of Caspase 3 activation and proteolytic PARP cleavage in cell lysates from cells treated with compounds of the invention would be consistent with compound-induced apoptotic cell death.

Effects of Laulimalide and Analogues on Tubulin Polymerization In Vitro.

One characteristic of the microtubule-stabilizing agents paclitaxel, discoldermolide, epothilones A and B, and eleutherobin is the ability of these agents to initiate the polymerization of tubulin in the absence of polymerization promoters, such as glycerol. For example, as reported by Mooberry et al. in US2002/0198256, at low micromolar concentrations more tubulin polymer was formed in the presence of paclitaxel, and the rate of polymerization was faster than was seen with equivalent concentrations of Laulimalide.

In certain embodiments, in assays to determine the effect of Laulimalide and analogues on tubulin polymerization, samples of the tubulin polymer formed may be examined by electron microscopy to determine whether the increase in turbidity measured during the polymerization experiments is due to the formation of microtubule-like polymers or the formation of other structures. Comparison under high magnification of the tubulin polymers formed in the presence of an inventive compound versus a known microtubule stabilizing agent (e.g., paclitaxel) allows an assessment of the effect of the inventive compound on tubulin polymerization.

1. A compound having the structure: 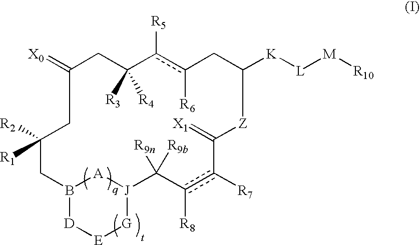
or pharmaceutically acceptable derivative thereof;
wherein R₁ and R₂ are independently hydrogen, halogen, or an aliphatic, alicyclic, heteroaliphatic, heteroalicyclic, aromatic or heteroaromatic moiety;
R₃ and R₄ are independently hydrogen, —OR^{3a} or —NR^{3a}R^{3b}, wherein at least one of R₃ and R⁴ is —OR^{3a} or —NR^{3a}R^{3b}, or R₃ and R₄ taken together with the carbon to which they are attached form a —C(═O)— or ═NR^{3c} moiety; wherein R^{3a} and R^{3b}, for each occurrence, is independently hydrogen, a protecting group, a prodrug moiety or an aliphatic, alicyclic, heteroaliphatic, heteroalicyclic, aromatic or heteroaromatic moiety; and R^{3c} is an aliphatic, alicyclic, heteroaliphatic, heteroalicyclic, aromatic or heteroaromatic moiety, or OR^{3d}; wherein R^{3d} is hydrogen or an aliphatic, alicyclic, heteroaliphatic, heteroalicyclic, aromatic or heteroaromatic moiety;
R₅ and R₆ are independently hydrogen, halogen, —CN, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form an alicyclic or heteroalicyclic moiety; wherein the carbon atoms to which R₅ and R₆ are attached may be connected by a single or double bond, as valency permits; and
wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or R₆, taken together with a substituent present on K, forms an alicyclic, heterocyclic, aromatic or heteroaromatic moiety;
R₇ and R₈ are independently absent, hydrogen, halogen, —CN, or an aliphatic, alicyclic, heteroaliphatic, heteroalicyclic, aromatic or heteroaromatic moiety, or R₇ and R₈, taken together, form an alicyclic, heteroalicyclic, aromatic or heteroaromatic moiety; wherein the carbon atoms to which R₇ and R₈ are attached may be connected by a single, double or triple bond, as valency permits;
R₉ₐ and R_{9b} are independently absent, hydrogen or an aliphatic, alicyclic, heteroaliphatic, heteroalicyclic, aromatic or heteroaromatic moiety, or one of R₉ₐ and R_{9b}, taken together with X₁, forms an alicyclic, heteroalicyclic, aromatic or heteroaromatic moiety;
R₁₀ is hydrogen or an aliphatic, alicyclic, heteroaliphatic, heteroalicyclic, aromatic or heteroaromatic moiety;
X₀ is CR^{X0a}R^{X0b}, O or NR^{X0a}; wherein R^{X0a} and R^{X0b} are independently hydrogen, a nitrogen protecting group, or an aliphatic, alicyclic, heteroaliphatic, heteroalicyclic, aryl or heteroaryl moiety;
X₁ is O, S or NR^{X1}, or X₁, taken together with one of R₉ₐ and R_{9b}, forms an alicyclic, heteroalicyclic, aromatic or heteroaromatic moiety; wherein R^{X1} is hydrogen, a nitrogen protecting group, or an aliphatic, alicyclic, heteroaliphatic, heteroalicyclic, aromatic or heteroaromatic moiety;
Z is O, NR^{Z1}, CR^{Z1}R^{Z2} or S, wherein R^{Z1} and R^{Z2} are independently hydrogen, halogen, a nitrogen protecting group, or an aliphatic, alicyclic, heteroaliphatic, heteroalicyclic, aromatic or heteroaromatic moiety;
K, L and M are independently absent, or a substituted or unsubstituted C₁₋₆alkylidene or C₂₋₆alkenylidene chain wherein up to two non-adjacent methylene units are independently optionally replaced by CO, CO₂, COCO, CONR^{P1}, OCONR^{P1}, NR^{P1}NR^{P2}, NR^{P1}NR^{P2}CO, NR^{P1}CO, NR^{P1}CO₂, NR^{P1}CONR^{P2}, SO, SO₂, NR^{P1}SO₂, SO₂NR^{P1}, NR^{P1}SO₂NR^{P2}, O, S, or NR^{P1}; wherein each occurrence of R^{P1} and R^{P2} is independently hydrogen, aliphatic, heteroaliphatic, aromatic, heteroaromatic or acyl, or a substitutent present on K, when present, and taken together with R₆, forms an alicyclic, heterocyclic, aromatic or heteroaromatic moiety;
A, B, D, E, G and J are independently connected by either a single or double bond, as valency permits, or A-B-D-E-G-J together represents an aromatic or heteroaromatic moiety; wherein B and J are independently N or CR^{Q1}; and A, D, E and G are independently C═O, CR^{Q1}R^{Q2}, NR^{Q1}, O, N or S; wherein each occurrence of R^{Q1} and R^{Q2} is independently absent, hydrogen, halogen, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or any two adjacent substituents on A, B, D, E, G and J, taken together, may represent an alicyclic, heteroalicyclic, aromatic or heteroaromatic moiety; and q and t are independently 0-2; wherein the sum q+t is 1-3; with the proviso that the compound is not one of: 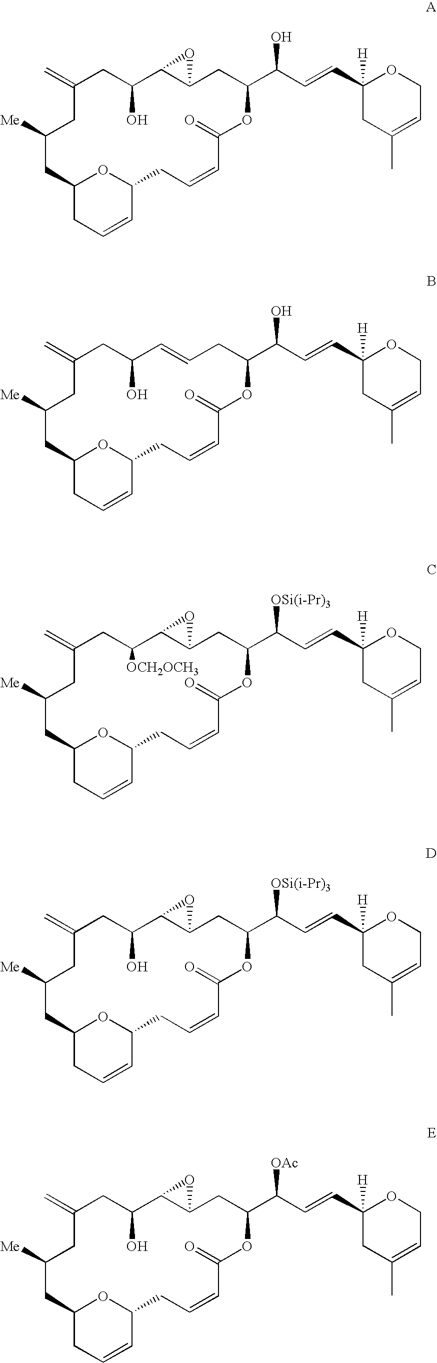 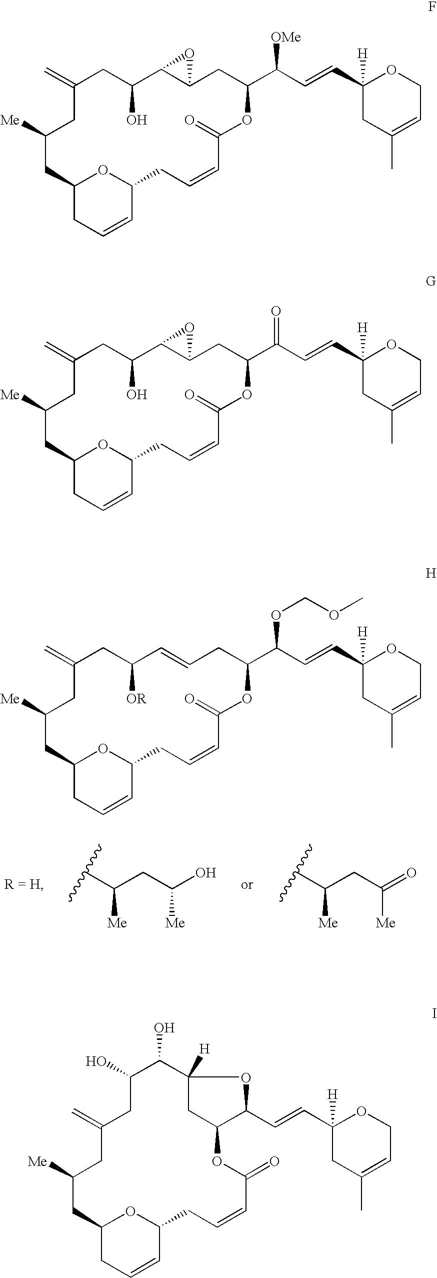 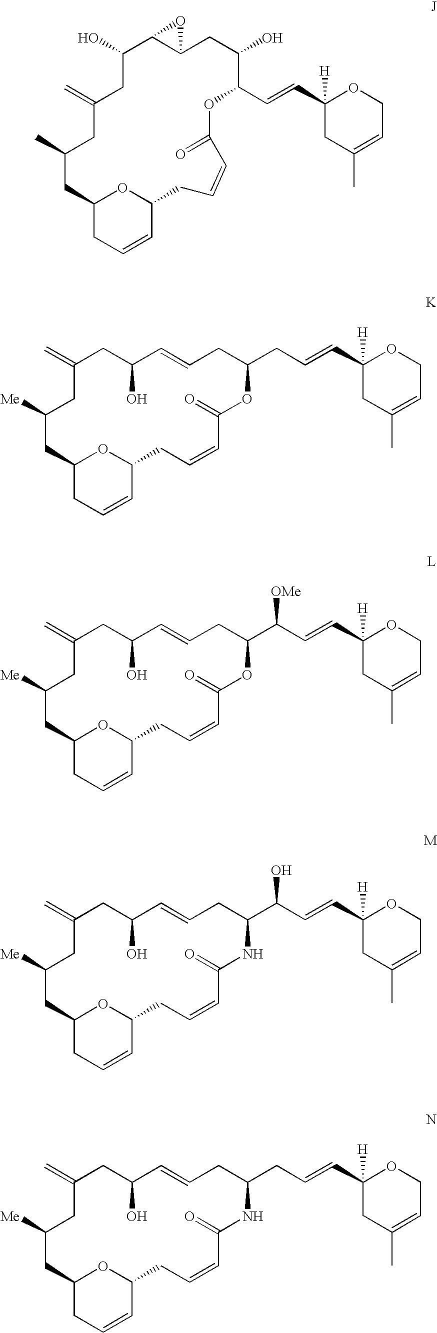 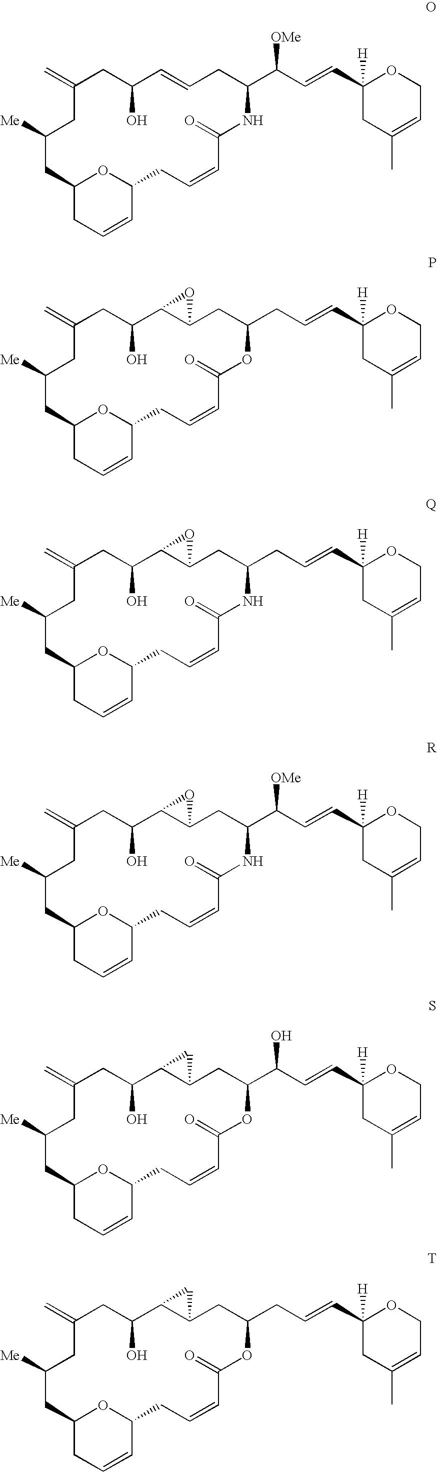 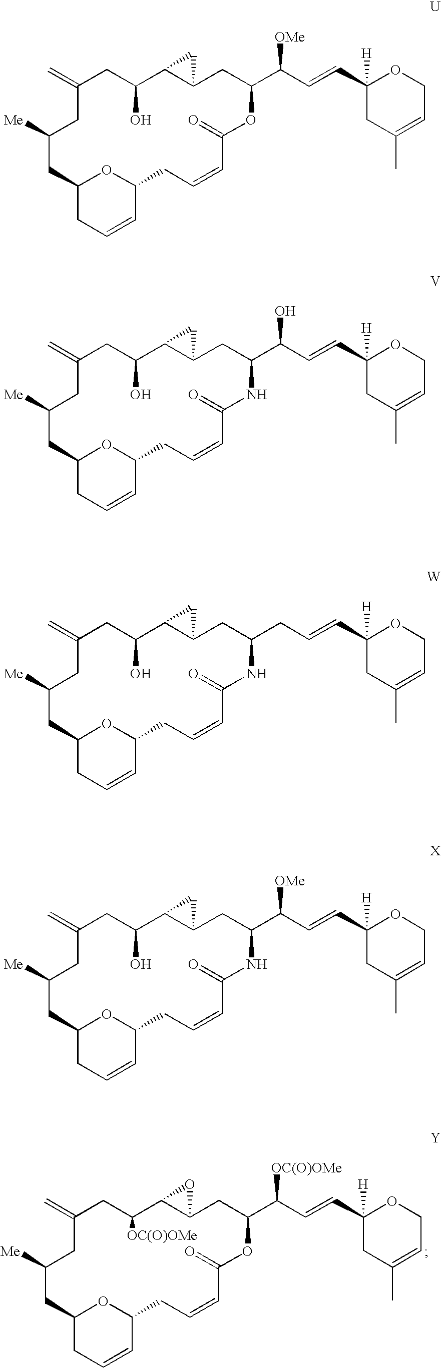
or any one of the compounds depicted on pages 107-111 and 114 of WO 03/076445.

2-81. (canceled)

82. A pharmaceutical composition comprising: a compound of claim 1; and a pharmaceutically acceptable carrier or diluent.

83-86. (canceled)

87. A method of inhibiting the growth of multidrug resistant cells in:
(a) a subject; or
(b) a biological sample; which method comprises administering to said subject, or contacting said biological sample with:
a) a composition according to claim 82; or
b) a compound having the structure: 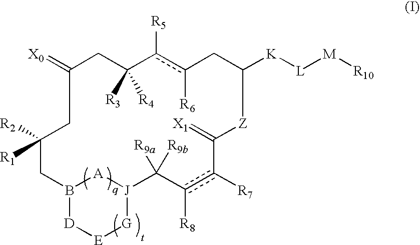
or pharmaceutically acceptable derivatives thereof; wherein R₁ and R₂ are independently hydrogen, halogen, or an aliphatic, alicyclic, heteroaliphatic, heteroalicyclic, aromatic or heteroaromatic moiety; R₃ and R₄ are independently hydrogen, —OR^{3a} or —NR^{3a}R^{3b}, wherein at least one of R₃ and R₄ is —OR^{3a} or —NR^{3a}R_{3b}, or R₃ and R₄ taken together with the carbon to which they are attached form a —C(═O)— or ═NR^{3c} moiety; wherein R^{3a} and R^{3b}, for each occurrence, is independently hydrogen, a protecting group, a prodrug moiety or an aliphatic, alicyclic, heteroaliphatic, heteroalicyclic, aromatic or heteroaromatic moiety; and R^{3c} is an aliphatic, alicyclic, heteroaliphatic, heteroalicyclic, aromatic or heteroaromatic moiety, or OR^{3d}; wherein R^{3d} is hydrogen or an aliphatic, alicyclic, heteroaliphatic, heteroalicyclic, aromatic or heteroaromatic moiety;
R₅ and R₆ are independently hydrogen, halogen, —CN, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form an alicyclic or heteroalicyclic moiety; wherein the carbon atoms to which R₅ and R₆ are attached may be connected by a single or double bond, as valency permits; and wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or R₆, taken together with a substituent present on K, forms an alicyclic, heterocyclic, aromatic or heteroaromatic moiety;
R₇ and R₈ are independently absent, hydrogen, halogen, —CN, or an aliphatic, alicyclic, heteroaliphatic, heteroalicyclic, aromatic or heteroaromatic moiety, or R₇ and R₈, taken together, form an alicyclic, heteroalicyclic, aromatic or heteroaromatic moiety; wherein the carbon atoms to which R₇ and R₈ are attached may be connected by a single, double or triple bond, as valency permits;
R₉ₐ and R_{9b} are independently absent, hydrogen or an aliphatic, alicyclic, heteroaliphatic, heteroalicyclic, aromatic or heteroaromatic moiety, or one of R₉ₐ and R_{9b}, taken together with X₁, forms an alicyclic, heteroalicyclic, aromatic or heteroaromatic moiety;
R₁₀ is hydrogen or an aliphatic, alicyclic, heteroaliphatic, heteroalicyclic, aromatic or heteroaromatic moiety;
X₁ is O, S or NR^{X1}, or X₁, taken together with one of R₉ₐ and R_{9b}, forms an alicyclic, heteroalicyclic, aromatic or heteroaromatic moiety; wherein R^{X1} is hydrogen, a nitrogen protecting group, or an aliphatic, alicyclic, heteroaliphatic, heteroalicyclic, aromatic or heteroaromatic moiety;
Z is O, NR^{Z1}, CR^{Z1}R^{Z2} or S, wherein R^{Z1} and R^{Z2} are independently hydrogen, halogen, a nitrogen protecting group, or an aliphatic, alicyclic, heteroaliphatic, heteroalicyclic, aromatic or heteroaromatic moiety;
K, L and M are independently absent, or a substituted or unsubstituted C₁₋₆alkylidene or C₂₋₆alkenylidene chain wherein up to two non-adjacent methylene units are independently optionally replaced by CO, CO², COCO, CONR^{P1}, OCONR^{P1}, NR^{P1}NR^{P2}, NR^{P1}NR^{P2}CO, NR^{P1}CO, NR^{P1}CO₂, NR^{P1}CONR^{P2}, SO, SO₂, NR^{P1}SO₂, SO₂NR^{P1}, NR^{P1}SO₂NR^{P2}, O, S, or NR^{P1}; wherein each occurrence of R^{P1} and R^{P2} is independently hydrogen, aliphatic, heteroaliphatic, aromatic, heteroaromatic or acyl, or a substitutent present on K, when present, and taken together with R₆, forms an alicyclic, heterocyclic, aromatic or heteroaromatic moiety;
A, B, D, E, G and J are independently connected by either a single or double bond, as valency permits, or A-B-D-E-G-J together represents an aromatic or heteroaromatic moiety; wherein B and J are independently N or CR^{Q1}; and A, D, E and G are independently C═O, CR^{Q1}R^{Q2}, NR^{Q1}, O, N or S; wherein each occurrence of R^{Q1} and R^{Q2} is independently absent, hydrogen, halogen, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, OC(═O), —C(═O)NR^{W2}, —NR^{W2}C(═O); wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or any two adjacent substituents on A, B, D, E, G and J, taken together, may represent an alicyclic, heteroalicyclic, aromatic or heteroaromatic moiety; and
q and t are independently 0-2; wherein the sum q+t is 1-3; provided that the method excludes contacting a hyperproliferative mammalian cell having a multiple drug resistant phenotype with a laulimalide compound, as defined in U.S. Pat. No. 6,414,015.

88. A method of treating or lessening the severity of a disease or condition associated with cell hyperproliferation in a subject, said method comprising a step of administering to said subject:
a) a composition according to claim 82; or
b) a compound having the structure: 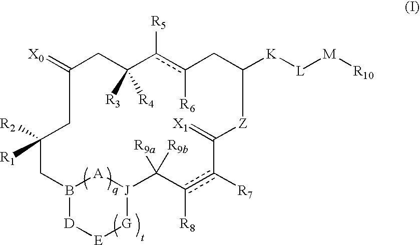
or pharmaceutically acceptable derivative thereof;
wherein R₁ and R₂ are independently hydrogen, halogen, or an aliphatic, alicyclic, heteroaliphatic, heteroalicyclic, aromatic or heteroaromatic moiety; R₃ and R₄ are independently hydrogen, —OR^{3a} or —NR^{3a}R^{3b}, wherein at least one of R₃ and R₄ is —OR^{3a} or —NR^{3a}R^{3b}, or R₃ and R₄ taken together with the carbon to which they are attached form a —C(═O)— or ═NR^{3c} moiety; wherein R^{3a} and R^{3b}, for each occurrence, is independently hydrogen, a protecting group, a prodrug moiety or an aliphatic, alicyclic, heteroaliphatic, heteroalicyclic, aromatic or heteroaromatic moiety; and R^{3c} is an aliphatic, alicyclic, heteroaliphatic, heteroalicyclic, aromatic or heteroaromatic moiety, or OR^{3d}; wherein R^{3d} is hydrogen or an aliphatic, alicyclic, heteroaliphatic, heteroalicyclic, aromatic or heteroaromatic moiety;
R₅ and R₆ are independently hydrogen, halogen, —CN, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); or R₅ and R₆, taken together, form an alicyclic or heteroalicyclic moiety; wherein the carbon atoms to which R₅ and R₆ are attached may be connected by a single or double bond, as valency permits; and wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or R₆, taken together with a substituent present on K, forms an alicyclic, heterocyclic, aromatic or heteroaromatic moiety;
R₇ and R₈ are independently absent, hydrogen, halogen, —CN, or an aliphatic, alicyclic, heteroaliphatic, heteroalicyclic, aromatic or heteroaromatic moiety, or R₇ and R₈, taken together, form an alicyclic, heteroalicyclic, aromatic or heteroaromatic moiety; wherein the carbon atoms to which R₇ and R₈ are attached may be connected by a single, double or triple bond, as valency permits;
R₉ₐ and R_{9b} are independently absent, hydrogen or an aliphatic, alicyclic, heteroaliphatic, heteroalicyclic, aromatic or heteroaromatic moiety, or one of R₉ₐ and R_{9b}, taken together with X₁, forms an alicyclic, heteroalicyclic, aromatic or heteroaromatic moiety;
R₁₀ is hydrogen or an aliphatic, alicyclic, heteroaliphatic, heteroalicyclic, aromatic or heteroaromatic moiety;
X₁ is O, S or NR^{X1}, or X₁, taken together with one of R₉ₐ and R_{9b}, forms an alicyclic, heteroalicyclic, aromatic or heteroaromatic moiety; wherein R^{X1} is hydrogen, a nitrogen protecting group, or an aliphatic, alicyclic, heteroaliphatic, heteroalicyclic, aromatic or heteroaromatic moiety;
Z is O, NR^{Z1}, CR^{Z1}Z^{Z2} or S, wherein R^{Z1} and R^{Z2} or S, wherein R^{Z1} and R^{Z2} are independently hydrogen, halogen, a nitrogen protecting group, or an aliphatic, alicyclic, heteroaliphatic, heteroalicyclic, aromatic or heteroaromatic moiety;
K, L and M are independently absent, or a substituted or unsubstituted C₁₋₆alkylidene or C₂₋₆alkenylidene chain wherein up to two non-adjacent methylene units are independently optionally replaced by CO, CO², COCO, CONR^{P1}, OCONR^{P1}, NR^{P1}NR^{P2}, NR^{P1}NR^{P2}CO, NR^{P1}CO, NR^{P1}CO₂, NR^{P1}CONR^{P2}, SO, SO₂, NR^{P1}SO₂, SO₂NR^{P1}, NR^{P1}SO₂NR^{P2}, O, S, or NR^{P1}; wherein each occurrence of R^{P1} and R^{P2} is independently hydrogen, aliphatic, heteroaliphatic, aromatic, heteroaromatic or acyl, or a substitutent present on K, when present, and taken together with R₆, forms an alicyclic, heterocyclic, aromatic or heteroaromatic moiety;
A, B, D, E, G and J are independently connected by either a single or double bond, as valency permits, or A-B-D-E-G-J together represents an aromatic or heteroaromatic moiety; wherein B and J are independently N or CR^{Q1}; and A, D, E and G are independently C═O, CR^{Q1}R^{Q2}, NR^{Q1}, O, N or S; wherein each occurrence of R^{Q1} and R^{Q2} is independently absent, hydrogen, halogen, an aliphatic, alicyclic, heteroaliphatic, heteroalicyclic, aromatic or heteroaromatic moiety, or is WR^{W1} wherein W is O, S, NR^{W2}, —C(═O), —S(═O), —SO₂, —C(═O)O—, —OC(═O), —C(═O)NR^{W2}, —NR^{W2}C(═O); wherein each occurrence of R^{W1} and R^{W2} is independently hydrogen, a protecting group, a prodrug moiety or an aliphatic, alicyclic, heteroaliphatic, heteroalicyclic, aromatic or heteroaromatic moiety, or, when W is NR^{W2}, R^{W1} and R^{W2}, taken together with the nitrogen atom to which they are attached, form a heteroalicyclic or heteroaryl moiety; or any two adjacent substituents on A, B, D, E, G and J, taken together, may represent an alicyclic, heteroalicyclic, aromatic or heteroaromatic moiety; and
q and t are independently 0-2; wherein the sum q+t is 1-3.

89-90. (canceled)
